(19) United States
(12) Patent Application Publication
(10) Pub. No.: US 2016/0008297 A1
(43) Pub. Date: Jan. 14, 2016

(54) COMPOUNDS FOR PREVENTING, REDUCING AND/OR ALLEVIATING ITCHY SKIN CONDITION(S)

(71) Applicants: Gerhard SCHMAUS, Höxter-Bosseborn (DE); Michael KROHN, Lorsch (DE); Dirk SOMBROEK, Darmstadt (DE); Lars Ole HAUSTEDT, Potsdam (DE); Grit KLUGE, Trebbin (DE); Oskar KOCH, Göttingen (DE); Antje KÖHLER, Holzminden (DE)

(72) Inventors: Gerhard SCHMAUS, Höxter-Bosseborn (DE); Michael KROHN, Lorsch (DE); Dirk SOMBROEK, Darmstadt (DE); Lars Ole HAUSTEDT, Potsdam (DE); Grit KLUGE, Trebbin (DE); Oskar KOCH, Göttingen (DE); Antje KÖHLER, Holzminden (DE)

(73) Assignee: B.R.A.I.N. Biotechnology Research And Information Network AG, Zwingenberg (DE)

(21) Appl. No.: 14/435,906

(22) PCT Filed: Aug. 23, 2012
(86) PCT No.: PCT/EP2012/066442

§ 371 Date: Sep. 24, 2015

(51) Int. Cl. A61K 31/12; A61K 8/35; A61Q 19/02; A61Q 19/00; A61Q 17/04; A61Q 5/00; A61Q 19/04; A61Q 15/00; A61Q 5/02; A61Q 11/00; A61Q 5/10; A61Q 5/12; A61Q 5/04; A23G 4/12; A61K 45/06

(52) U.S. Cl. CPC A61K 31/12; A61K 45/06; A61K 8/35; A61Q 19/02; A61Q 19/004; A61Q 17/04; A61Q 5/006; A61Q 19/04; A61Q 15/00; A61Q 5/02; A61Q 11/00; A61Q 5/00; A61Q 5/10; A61Q 5/12; A61Q 19/007; A61Q 19/00; A61Q 5/04; A23G 4/12

(57) ABSTRACT

The present invention primarily relates to the use of one or more specific compounds and/or one or more respective salt(s) thereof for preventing, reducing or alleviating itchy skin condition(s), and/or as PAR-2 antagonist. Furthermore, the present invention relates to compositions (products or, respectively, formulations), in particular for topical administration, preferably cosmetic or pharmaceutical compositions, in particular for preventing, reducing or alleviating one or more itchy skin conditions and/or for providing a PAR-2 antagonistic effect, comprising or consisting of an effect amount of such compound(s) and/or salt(s) and one or more cosmetically and/or pharmaceutically acceptable carriers.

The present invention primarily relates to the use of one or more compounds of the general formula 1 and/or one or more respective salt(s) thereof

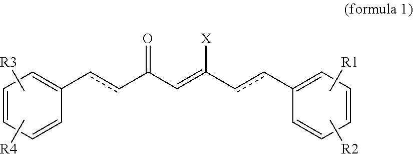

with R1 to R4 and X having the meaning as defined herein below, in particular as defined in the claims, for preventing, reducing or alleviating itchy skin condition(s), and/or as PAR-2 antagonist.

Furthermore, the present invention relates to compositions (products or, respectively, formulations), in particular for topical administration, preferably cosmetic or pharmaceutical compositions, in particular for preventing, reducing or alleviating one or more itchy skin conditions and/or for providing a PAR-2 antagonistic effect, comprising or consisting of an effect amount of compound(s) of formula 1 and/or respective salt(s) thereof and one or more cosmetically and/or pharmaceutically acceptable carriers.

The present invention also relates to cosmetic, preferably non-therapeutic, or therapeutic methods

a) for preventing, reducing or alleviating one or more itchy conditions, and/or
b) for providing a PAR-2 antagonistic effect,
wherein compound(s) of formula 1 and/or respective salt(s) thereof or a composition according to the present invention is/are used.

Further aspects of the present invention become apparent by studying the following specification, the examples described herein as well as, in particular, the attached claims.

In particular in the cosmetics and pharmaceuticals industry, there is a constant need for agents having a skin soothing, in particular an itch-reducing or -alleviating action. The skin, in particular the epidermis, as a barrier organ of the human organism is subjected to external influences to a particular extent. Many intrinsic factors (e.g. genetic predisposition) and extrinsic factors (e.g. damage to the skin barrier, dry, especially itchy winter skin, itch-inducing substances) can lead to skin itch, for example skin itch of the scalp.

In connection with this invention, itchy skin is preferably to be understood as meaning any change to the skin which induces sensorial malaise in humans and/or is characterized by the symptoms as dry, reddened or scaly skin. Skin itch can include, be caused or, respectively, be accompanied by phenomenologically different skin states, such as delicate skin, sensitive skin, including sensitive scalp, easily injured skin, atopic skin, irritated skin or inflamed skin, which may manifest itself in an in each case higher severity in itch. In addition, itch is furtheron to be understood as meaning any change to the skin which induces the immediate desire to scratch. Itch is one of the most common disturbing skin conditions and can have high influence on life quality. Itch can be caused e.g. by rapid changes in temperature and/or wind, by specific systemic medication or by disturbed epidermal barrier. It is clearly on the one hand a neurological phenomenon with diverse receptors (e.g. histamine 1-4-, NK1-, TNF-alpha-, PAR-2-receptors) and respective mediators (histamines 1-4, Substance P, TNF-alpha, Tryptase, Trypsin and peptides like e.g. SLIGR and SLIGRL) being involved but on the other hand itch also clearly correlates with an impaired epidermal barrier. There is upcoming evidence that PAR-2 signaling is involved in e.g. atopic dermatitis, predisposed atopic dermatitis and dry skin, suffering from itchy skin. As described by Steinhoff et al (J. Neuroscience, Vol. 23(15), p. 6176-6180, 2003) patients suffering from atopic dermatitis showed an up to fourfould increase of the PAR-2 agonist tryptase. Furtheron, the PAR-2 receptor was marketly enhanced on primary afferent nerve fibres in skin biopsies of atopic dermatitis patients.

In about 10-20% of the population of industrial countries, with an increasing trend, atopy is to be observed. Atopy can manifest itself as dry itchy skin, predisposed atopic dermatitis or in severe disease states atopic dermatitis. The different severity states are associated with different severity of damaged skin and scalp barrier and skin is frequently itchy. Besides intrinsic, genetic factors, there are also extrinsic factors as for example environmental pollution, UV stress, diverse chemical substances that may cause atopy. A too frequent usage of soap, shampoos or other hair care and/or coloring compositions, in general surfactants causing defattening and drying out of skin and scalp may also support the generation of an atopy and consequently itchy skin conditions.

There are only a view active compounds known, e.g. mast cell stabilizers like sodium chromoglycate, NK1 receptor antagonists like aprepitant or a number of plant extracts e.g. from oat, having an itch reducing action and that are already employed in the technical fields referred to, but alternatives nevertheless continue to be sought.

In connection with the invention described herein itch-reducing action is preferably to be understood as meaning the modulation, soothing, reduction, alleviating, elimination or prevention of itchy skin (for example, but not limited to, skin of the scalp), in particular that of the uncomfortable itch feeling that causes exhaustive scratching and in serious cases as a consequence injury of skin.

In the search for suitable agents it has to be remembered that the substances used should be toxicologically acceptable, tolerated well by the skin and stable (in particular in conventional cosmetic and/or pharmaceutical formulations), should preferably have the lowest possible intrinsic odour and the lowest possible intrinsic colour and be inexpensive to prepare. In accordance with the persistent trend towards natural active compounds, novel active compounds of natural, in particular plant origin are sought in particular.

Persons skilled in the art have already addressed the problem of skin itch and have described, e.g. the skin itch-reducing properties of hydrocortisone or anti-histaminics. However, hydrocortisone, being considered as a potent anti-inflammatory agent, shows only poor itch-reducing activity while used for the treatment of conditions associated with dry scalp and/or skin, predisposed atopic skin or atopic dermatitis and skin and scalp barrier deficiencies. In this regard, it is to be noted that anti-inflammatory action does not necessarily go along with itch-reducing action. Regarding anti-histaminics, the scientific literature mentions that they are effective in case of e.g. insect bites, however they show only poor clinical efficacy in the treatment of itchy skin conditions associated with e.g. atopic dermatitis, predisposed atopic dermatitis and dry, itchy frequently winter skin, exposed to low and drying out temperature conditions.

A primary object of the present invention was thus to provide suitable agents having, preferably improved, itch-reducing action, and, preferably, being toxicologically acceptable, tolerated well by the skin and being stable in conventional cosmetic and/or pharmaceutical formulations, having the lowest possible intrinsic odour and the lowest possible intrinsic colour and preferably being inexpensive to prepare.

Further objects of the present invention can be derived from the following specification, the examples described herein as well as, in particular, the attached claims.

The primary object of the invention is achieved by the use, preferably non-therapeutic use, of
a compound of formula 1 or a respective salt thereof

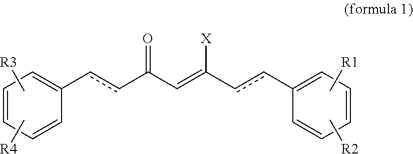

with R1, R2, R3 and R4 independently from each other meaning —OH, —OR or —OCOR, with R meaning an alkyl group comprising 1-12 carbon atoms,
with [symbol] meaning (in each case, independent from each other) an optional additional bond, wherein, according to a preferred aspect of the invention, only one or none of the dotted lines ( [symbol] ) means an additional bond, and
with X meaning —H or —OH, wherein in the case of X meaning —OH the respective substance is preferably present or, respectively, used in a keto-enol-equilibrium with the corresponding diketone,
   or
a mixture comprising or consisting of one, two (cf. above: for example, in the case of X meaning —OH in a first compound of formula 1, wherein said compound is used in a keto-enol-equilibrium with the corresponding diketone) or more compounds of formula 1 and/or one, two or more respective salts thereof
   a) for preventing, reducing or alleviating one or more itchy skin, in particular human skin (such as, but not limited to, skin of the scalp), conditions, and/or
   b) as PAR-2 antagonist.

Accordingly, the present invention also relates to a compound of formula 1 or a respective salt thereof

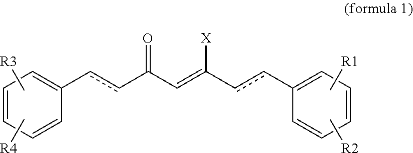

with R1, R2, R3 and R4 independently from each other meaning —OH, —OR or —OCOR, with R meaning an alkyl group comprising 1-12 carbon atoms,
with each [symbol] independently from each other meaning an optional additional bond, wherein, according to a preferred aspect of the invention, only one or none of the dotted lines ( [symbol] ) means an additional bond, and
with X meaning —H or —OH, wherein in the case of X meaning —OH the respective substance is preferably present or, respectively, used in a keto-enol-equilibrium with the corresponding diketone,
   or
   a mixture comprising or consisting of one, two or more compounds of formula 1 and/or one, two or more respective salts thereof
   a) for use in the treatment or prevention of one or more itchy skin conditions, and/or
   b) for use as PAR-2 antagonist.

Preferably, the compound, salt or mixture is suitable for topical application, in particular for being used as cosmetical and/or pharmaceutical composition or, respectively, as a part thereof.

The itch-reducing action according to the invention is preferably based on soothing of the skin via inhibition of the PAR-2 receptor or, respectively, via the PAR-2 antagonistic action of compounds and mixtures described herein, preferably via the direct PAR-2 antagonistic action thereof. Particularly preferably, the (itch-reducing) action according to the invention is based on (direct) PAR-2 antagonistic activity, wherein the PAR-2 antagonist(s) (i.e. the compound(s) to be used according to the invention as described herein) mediate its/their effect(s) by binding to the active site or to allosteric site(s) on PAR-2 receptors, or interact at unique binding site(s), which are usually not involved in the biological regulation of the receptors activity. According to a preferred aspect of the present invention, itch-reducing action (as described herein) does not refer to or comprise or, respectively, is not (at least not primarily) based on an anti-inflammatory effect. As mentioned above, anti-inflammatory action does not necessarily go along with itch-reducing action.

Advantageously, the antagonistic effect on PAR-2 (as described herein) can also lead to strengthening of the (epidermal) skin barrier and/or to lightening of the skin. I.e., preferebly the compounds, mixtures and composition according to the present invention (as described herein) are (also) used for strengthening of the (epidermal) skin barrier and/or as a skin lightener.

The proteinase-activated receptor-2 (PAR-2) belongs to a family of four G-protein coupled receptors (GPCR's). PAR-2 signaling is irreversibly induced by various serine proteinases including mast cell tryptase, trypsine, kallikreins and others by cleavage and unmasking of the extracellular N-terminal domain, a mechanism termed tethered ligand activation (see literature: e.g. U. J. K. Soh et al., British Journal of Pharmacology, 160:191-203, 2010 or S. E. Lee et al., Yonsei Med. J., 51(6): 808-822, 2010). PARs are unique among GPCRs, in that their activation occurs by irreversible proteolytic cleavage of the N-terminus. Trypsin-like serine proteases cleave a cryptic ligand sequence in the N-terminus of PAR-2 (SKGR|SLIGR). In consequence the exposed N-terminus binds to the surface of the second extracellular loop of PAR-2 and triggers signaling cascades typically mediated by Gαq, Gα12/13 and Gαqi/o. Trypsin induced tethered ligand activation unmasks the peptide N-terminal sequence SLIGRL and e. g. Gαq-Protein triggered signaling cascade, and rise of intracellular calcium is induced. Thus, PAR-2 activation can also be triggered via application of small agonistic peptides like SLIGR and SLIGRL. Interaction of an activated PAR-2 with its accompanying heterotrimeric (αβγ) G-protein leads to the nucleotide exchange (GDP to GTP) at the α-subunit of the G-protein. The activated G-protein dissociates into a Gα-GTP subunit and the βγ-subunit complex, which each can interact with different signal transducer proteins. The Gαq-GTP subunit can specifically interact and activate phospholipase C, which in turn catalyses the hydrolysis of membrane-bound phosphatidylinositol(4,5)-biphosphate [PtdIns (4,5)P2] into diacylglycerol (DAG) and inositol(1,4,5)-triphosphate (IP3). IP3 formation results in release of Ca2+, which can be detected with fluorescence-sensitive dyes. At the same time IP3 also activates protein kinase C (PKC), which can be measured, e.g., at the level of gene transcription using appropriate reporter gene assays.

Skin exposure to exogenous applied PAR-2 activating proteases and agonistic peptides like SLIGR or SLIGRL can induce itch via PAR-2. A scientific study reported that mice, overexpressing epidermal KLK 7 displayed massive itchy behaviour (M. Steinhoff et al., J. Invest, Dermatology, Vol. 126, p. 1705-1718, 2006). Another scientific study reported that PAR-2 agonistic (activating) trypsin induced scratching behaviour in mice was inhibited by a PAR-2 antagonistic (inhibiting) peptide, suggesting a significant role of PAR-2 activation via serine proteases like tryptase in itch perception (R. Costa et al.; Br. J. Pharmacol., Vol. 154, p. 1094-1103, 2008). Consequently, it is suggested that PAR-2 antagonistic peptides might be a promising therapeutic tool for the prevention and/or treatment of itchy skin conditions thus helping to break the viscious itch-scratch cycle observed in itchy skin conditions.

In the context of the present invention “antagonistic activity” refers to a pharmaceutical and/or cosmetic active inhibition of the PAR-2 related bioactivity. According to a preferred embodiment, the compound(s) of formula 1 and/or salt(s) thereof or a composition according to the invention as described herein is/are used in an antagonistically effective amount, i.e. an amount sufficient to modulate, and preferably reduce by at least 20 percent, more preferably at least 50 percent, most preferably by at least 80 percent, the PAR-2 receptor activity, preferably measured as described in example 2.

As mentioned before, PAR-2 antagonistic peptides are described in the scientific literature as tool to investigate PAR-2 signaling pathways. However they are just occasionally described as a clinically efficient therapeutic tool for the prevention and or treatment of itchy skin conditions. It is commonly known that peptides are difficult to synthesize in large quantities. Furthermore, they are also known to have very poor ability to penetrate skin and, thus, consequently also to have only a poor ability to antagonise PAR-2 receptor signalling located on skin cells and sensory nerve endings and therefore only poor human clinical efficacy.

Due to the fact that PAR-2 antagonising peptides have certain disadvantages in clinical applications and the accumulating scientific evidence that activation of PAR-2 is involved in human skin abnormalies like inter alia atopic dermatitis, dry itchy skin, skin barrier deficiency, the inventors have set up a research program to identify and develop structurally new PAR-2 receptor antagonists for preventing, reducing and/or alleviating itchy skin condition(s). As there were only a few PAR-2 antagonistic lead structures known, most of them peptides, a high throughput screening was performed. Evaluation of stability and human safety properties of hit candidates resulted in a plurality of suitable compounds of formula 1 (as described herein) and respective salts thereof. The clinical skin soothing anti-itch efficacy was shown in in vivo studies on subjects suffering from itchy skin.

Preferably, the or, respectively, one, more or all of the compounds of formula 1 are partially hydrogenated.

Particularly preferred is the use, a compound, a respective salt thereof or a mixture according to the invention, wherein the compound of formula 1 or the respective salt thereof or, respectively, one, more or all compounds of formula 1 or respective salts thereof is/are selected from the group consisting of compounds of formula 2 and respective salts thereof

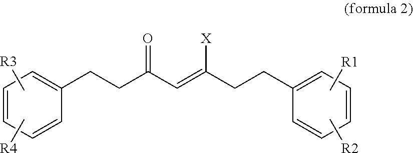

with R1, R2, R3 and R4 independently from each other meaning —OH, —OR or —OCOR, with R meaning an alkyl group comprising 1-12 carbon atoms, and
with X meaning —H or —OH, wherein in the case of X meaning —OH the respective substance is preferably present or, respectively, used in a keto-enol-equilibrium with the corresponding diketone.

Advantageously, these compounds have no or only low intrinsic colour.

Particularly preferred in connection with the present invention are compounds of formula 3 and 4 or respective salts thereof:

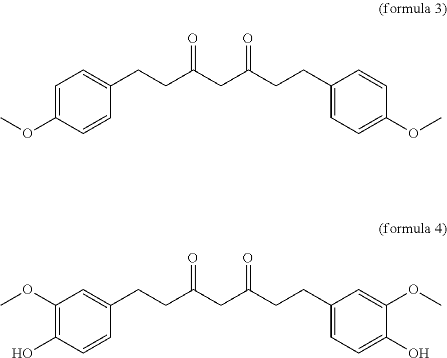

Thus, according to a preferred embodiment of the present invention, the compound of formula 1 or 2 or the respective salt thereof or, respectively, one compound of formula 1 or 2 or one respective salt thereof is a compound of formula 3 or a respective salt thereof.

Also preferred is

a use, a compound, a respective salt thereof or a mixture according to the invention (as described herein), wherein the compound of formula 1 or 2 or the respective salt thereof or, respectively, one compound of formula 1 or 2 or one respective salt thereof is a compound of formula 4 or a respective salt thereof,
or
a use or a mixture as described above (wherein the compound of formula 1 or 2 or the respective salt thereof or, respectively, one compound of formula 1 or 2 or one respective salt thereof is a compound of formula 3 or a respective salt thereof),
wherein—in addition—one (further) compound of formula 1 or 2 or one respective salt thereof is a compound of formula 4 or a respective salt thereof.

Preferred in connection with the present invention are furtheron plant extracts or fractions thereof comprising compound(s) of the general formula 1 and/or preferred partially hydrogenated compounds, preferably of the general formula 2, present in or, respectively, derived from plant species like Curcuma aromatica, Curcuma longa, Curcuma domestica, Curcuma xanthorrhiza, Curcuma zedoaria (zedoary), other Curcuma species and subspecies, Alpinia officinarum, Alpinia conchigera, Alpinia blepharocalyx, other Alpinia species and subspecies, Zingiber cassumunar, Aframomum letestuianum, Cyathostemma viridiflorum, Garuga pinnata, Sophora leachiana, Nostoc sp. strain Lukesova 27/97, herewith mentioning that this are just just a few examples.

Plant extracts comprising compounds of formulae 1 may be obtained using conventional methods including, but not limited to, direct extraction of the plant material by grinding, macerating, pressing, squeezing, mashing, centrifuging, and/or processes such as cold percolation, agitation/distillation, microwave assisted extraction, sonication, supercritical/subcritical CO2 compressed gas extraction with or without polar modifiers, pressurized solvent extraction, accelerated solvent extraction, pressurized or normal hot water extraction, surfactant assisted pressurized hot water extraction, oil extraction, membrane extraction, Soxhlet extraction, the gold finger distillation/extraction and the like. Any of a variety of solvents including polar solvents, non-polar solvents, or combinations of two or more thereof may be used in methods of comprising solvent extraction. In certain more preferred embodiments, the extract is prepared using a solvent comprising methanol, ethanol, or a combination thereof with or without presence of water. In certain preferred embodiments, the extract may be further refined by charcoal (also referred to as active carbon) treatment. In a special application form, catalytic hydrogenation of plant extracts, specific fractions from plant extracts with the aim to partially hydrogenate aliphatic double bonds present in compounds of general formula 1 is a further, highly specific means to produce plant extracts or plant extract fractions comprising after catalytic hydrogenation treatment partially hydrogenated compounds of general formula 2, also bearing the trivial name “tetrahydrodiarylheptanoids”. Specific compounds are e.g 1,7-Bis(4-methoxyphenyl)-3,5-heptanedione (formula 3) and 1,7-Bis-(4-hydroxy-3-methoxyphenyl)-3,5-heptanedione (formula 4).

Also preferred is a use or a mixture according to the invention, wherein the mixture additionally comprises one or more (further) substances (in particular substances suitable for cosmetic and/or dermatological applications) for preventing, reducing or alleviating itchy skin condition(s) and/or one or more skin irritation-reducing agents, in particular one or more substances selected from the group consisting of anti-inflammatory agents, physiological cooling agents and compounds that alleviate reddening, preferably wherein the one or more additional substances is/are selected from the group consisting of (the additional substances being substances other than compounds according to formula 1 or respective salts thereof):
(i) (further) anti-itch compounds,
(ii) steroidal anti-inflammatory substances of the corticosteroid type, in particular hydrocortisone, hydrocortisone derivatives such as hydrocortisone 17-butyrate, dexamethasone, dexamethasone phosphate, methylprednisolone or cortisone,
(iii) non-steroidal anti-inflammatory substances, in particular oxicams such as piroxicam or tenoxicam, salicylates such as aspirin, disalcid, solprin or fendosal, acetic acid derivatives such as diclofenac, fenclofenac, indomethacin, sulindac, tolmetin or clindanac, fenamates such as mefenamic, meclofenamic, flufenamic or niflumic, propionic acid derivatives such as ibuprofen, naproxen or benoxaprofen, pyrazoles such as phenylbutazone, oxyphenylbutazone, febrazone or azapropazone,
(iv) natural or naturally occurring anti-inflammatory substances or substances that alleviate reddening and/or itching, in particular extracts or fractions from camomile, Aloe vera, Commiphora species, Rubia species, willow, willow-herb, oats, calendula, arnica, St John's wort, honeysuckle, rosemary, Passiflora incarnata, witch hazel, ginger or Echinacea, or single active compounds thereof,
(v) alpha-bisabolol, apigenin, apigenin-7-glucoside, gingerols, shogaols, gingerdiols, dehydrogingerdiones, paradols, natural avenanthramides, non-natural avenanthramides, preferably dihydroavenanthramide D, boswellic acid, phytosterols, glycyrrhizin, glabridin and licochalcone A, preferably in the form of pure substances,
(vi) skin care agents, preferably skin moisture retention regulators or skin repair agents, preferably selected from the group consisting of sodium lactate, urea and derivatives, glycerol, propylene glycol, 1,2-pentanediol, 1,2-hexanediol and 1,2-octanediol, collagen, elastin or hyaluronic acid, diacyl adipates, petrolatum, urocanic acid, lecithin, allantoin, panthenol, phytantriol, lycopene, (pseudo-)ceramides (preferably Ceramide 2, hydroxypropyl bispalmitamide MEA, cetyloxypropyl glyceryl methoxypropyl myristamide, N-(1-hexadecanoyl)-4-hydroxy-L-proline (1-hexadecyl) ester, hydroxyethyl palmityl oxyhydroxypropyl palmitamide), glycosphingolipids, cholesterol, phytosterols, chitosan, chondroitin sulfate, lanolin, lanolin esters, amino acids, vitamin E and derivatives (preferably tocopherol, tocopheryl acetate), alpha-hydroxy acids (preferably citric acid, lactic acid, malic acid) and derivatives thereof, mono-, di- and oligosaccharides, preferably glucose, galactose, fructose, mannose, laevulose and lactose, polysugars, such as β-glucans, in particular 1,3-1,4-β-glucan from oats, alpha-hydroxy-fatty acids, triterpenic acids, such as betulic acid or ursolic acid, and algae extracts or single active compounds thereof,
(vii) physiological cooling agents, preferably selected from the group consisting of menthone glycerol acetal, menthyl lactate preferably l-menthyl lactate, in particular l-menthyl l-lactate), menthyl ethyl oxamate, substituted menthyl-3-carboxylic acid amides (e.g. menthyl-3-carboxylic acid N-ethylamide, N^{α}-(L-menthanecarbonyl)glycine ethyl ester, 2-isopropyl-N-2,3-trimethylbutanamide, substituted cyclohexanecarboxylic acid amides, 3-menthoxypropane-1,2-diol, 2-hydroxyethyl menthyl carbonate, 2-hydroxypropyl menthyl carbonate, N-acetylglycine menthyl ester, isopulegol, menthyl hydroxycarboxylic acid esters (e.g. menthyl 3-hydroxybutyrate), monomenthyl succinate, monomenthyl glutarate, 2-mercaptocyclodecanone, menthyl 2-pyrrolidin-5-onecarboxylate, 2,3-dihydroxy-p-menthane, 3,3,5-trimethylcyclohexanone glycerol ketal, 3-menthyl 3,6-di- and -trioxaalkanoates, 3-menthyl methoxyacetate and icilin, and
(viii) histamine receptor antagonists, serine protease inhibitors (e.g. of Soy extracts), TRPV1 antagonists (e.g. 4-t-Butylcyclohexanol), NK1 antagonists (e.g. Aprepitant, Hydroxyphenyl Propamidobenzoic Acid), cannabinoid receptor agonists (e.g. Palmitoyl Ethanolamine) and TRPV3 antagonists.

However, it is particularly preferred that the total amount of compound(s) of formula 1 and/or respective salt(s) thereof in the mixture is sufficient to a) prevent, reduce or alleviate one or more itchy skin conditions, and/or to b) provide a PAR-2 antagonistic effect.

As may be apparent from the above description, a further aspect of the present invention relates to a composition, in particular for topical administration, preferably a cosmetic or pharmaceutical composition, in particular for preventing, reducing or alleviating one or more itchy skin conditions and/or for providing a PAR-2 antagonistic effect, comprising or consisting of
a) a total amount of 0.01 to 10.0 wt. %, preferably of 0.01, in particular of 0.05, to 5.0 wt. %, more preferably of 0.01, in particular of 0.1, to 2.5 wt. %, of one or more compounds selected from the group consisting of compounds of formula 1, 2, 3 and 4,

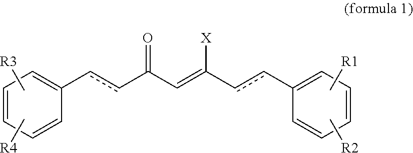

with R1, R2, R3 and R4 independently from each other meaning —OH, —OR or —OCOR, with R meaning an alkyl group comprising 1-12 carbon atoms,
with [symbol] meaning an optional additional bond, wherein, according to a preferred aspect of the invention, only one or none of the dotted lines ( [symbol] ) means an additional bond, and
with X meaning —H or —OH,

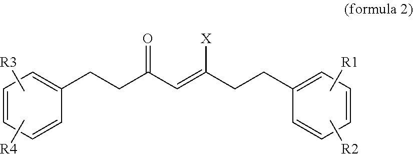

with R1, R2, R3 and R4 independently from each other meaning —OH, —OR or —OCOR, with R meaning an alkyl group comprising 1-12 carbon atoms, and
with X meaning —H or —OH,

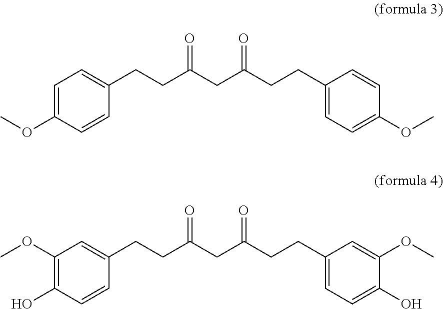

and/or one or more respective salts thereof, preferably one or more cosmetically and/or pharmaceutically acceptable salts thereof, in particular Na⁺, K⁺, NH₄⁺, Mg²⁺ or Ca²⁺ salts, based on the total weight of the composition, and
b) one or more cosmetically and/or pharmaceutically acceptable carriers, preferably cosmetically and/or pharmaceutically acceptable carriers other than water, more preferably carriers selected from the group consisting of glycols, aliphatic esters, in particular aliphatic esters showing good solubilising properties for one, more or all of the substances of component a), preferably polyethyleneglycol esters and polyethyleneglycol ethers or mixtures thereof, in particular cosmetically and/or pharmaceutically acceptable carriers for enhancing the bioavailability of one, more or all of the substances of component a).

Of course, the above explanations regarding preferred compounds and/or salts to be used in connection with the present invention or preferred effects/uses thereof also apply to a composition according to the present invention.

Particularly preferred in connection with all aspects of the present invention are combinations of compound(s) of formula 1, in particular of formula 2, preferably of formula 3, and polyethylene glycol ester(s) of formula 5 and/or polyethyleneglycol ether(s) of formula 6 (as described herein below). Thus, the or, respectively, one, more or all cosmetically and/or pharmaceutically acceptable carriers (as described above) are preferably selected from the group consisting of polyethylene glycol ester(s) of formula 5 and/or polyethyleneglycol ether(s) of formula 6 (as described herein below).

Compositions according to the present invention, in particular topical cosmetic compositions, are particularly suitable for skin soothing, in particular for preventing, reducing and/or alleviating itchy skin sensations.

The invention also provides a pharmaceutical composition, preferably a medicament, in particular for topical administration, preferably for preventing, reducing or alleviating one or more itchy skin conditions and/or for providing a PAR-2 antagonistic effect, comprising one or more compounds selected from the group consisting of compounds of formula 1, 2, 3 and 4,

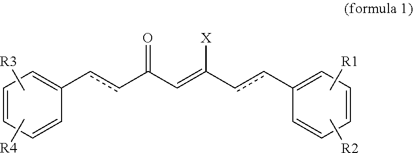

with R1, R2, R3 and R4 independently from each other meaning —OH, —OR or —OCOR, with R meaning an alkyl group comprising 1-12 carbon atoms,
with [symbol] meaning an optional additional bond, wherein, according to a preferred aspect of the invention, only one or none of the dotted lines ( [symbol] ) means an additional bond, and
with X meaning —H or —OH,

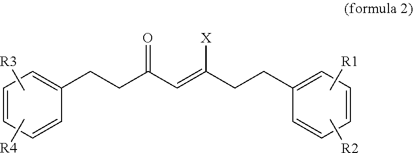

with R1, R2, R3 and R4 independently from each other meaning —OH, —OR or —OCOR, with R meaning an alkyl group comprising 1-12 carbon atoms, and
with X meaning —H or —OH,

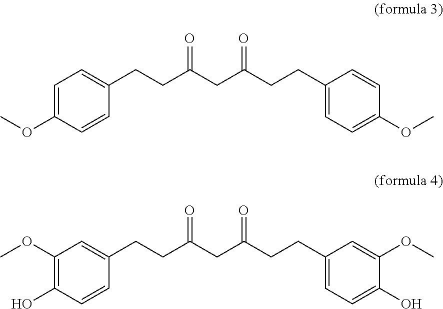

and/or one or more respective salts thereof, preferably one or more cosmetically and/or pharmaceutically acceptable salts thereof, in particular Na⁺, K⁺, NH₄⁺, Mg²⁺ or Ca²⁺ salts,
preferably in a total amount of 0.01 to 10.0 wt. %, preferably of 0.01, in particular of 0.05, to 5.0 wt. %, more preferably of 0.01, in particular of 0.1, to 2.5 wt. %, based on the total weight of the composition.

Again, the above explanations regarding preferred compounds and/or salts to be used in connection with the present invention or preferred effects/uses thereof also apply to a pharmaceutical composition according to the present invention.

Such a pharmaceutical composition can be employed in the field of human medicine against a large number of topical itchy conditions and diseases, such as, for example, urticaria, contact dermatitis, atopic dermatitis, insect bites but also other diseases associated with itch like e.g. renal diseases and AIDS. Also included are tooth and gum inflammation, such as parodontosis, as PAR-2 activation plays a role here too.

The inventors found out that compounds of the general formula 1 are very effective, highly specific PAR-2 antagonists in vitro as well as in vivo (cf. examples 1 to 3).

It was further observed by the inventors that compounds of the formula 1 are solid at 20° C. and are—under certain conditions—hardly to be solubilised. Preferably, this issue is to be kept in mind when handling and storing compounds of formula 1, in particular when using these compounds in (topical) cosmetic compositions and products, in particular compositions comprising water in an amount of 10 wt. % or more, based on the total weight of the formulation or product, or comprising a water and an oil phase (e.g. O/W- or W/O-emulsions). Without wishing to be bound by theory, it is assumed by the inventors that a combination with cosmetically acceptable solubilisers mentioned below improves the solubility of compounds of the general formula 1 and thereby improves the skin soothing, itch-reducing properties.

Further, it was also found by the inventors that these preferred carriers (as described below) can avoid the tendency of compounds of the general formula 1 to (re-)crystallize out of (topical, preferably cosmetic) compositions.

Thus, a composition according to the invention (as described above), wherein the or, respectively, one, more or all cosmetically and/or pharmaceutically acceptable carrier(s) is/are selected from the group consisting of
(i) polyethylene glycol esters of formula 5

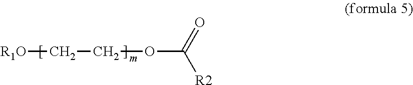

wherein m=3, 4, 5, 6 or 7,
R₁ means H or

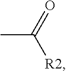

and
R2 means a branched or unbranched alkyl group, in which the number of carbon atoms preferably amounts to 8,
polyethyleneglycol ethers of formula 6

R₃OCH₂—CH₂ₙOR₄ (formula 6)
wherein n=7, 8, 9, 10, 11, 12, 13, 14, 15, 16, 17, 18, 19, 20, 21, 22, 23, 24, 25, 26, 27, 28, 29 or 30, and
R₃ and R₄ in each case, mutually independently, mean H or a saturated or unsaturated, branched or unbranched alkyl group, in which the number of carbon atoms preferably amounts to 10, 11, 12, 13, 14 or 15,
and
mixtures of polyethylene glycol esters of formula 5 and polyethyleneglycol ethers of formula 6,
preferably wherein the ratio of total amount of polyethylene glycol esters of formula 5 and the total amount of polyethyleneglycol ethers of formula 6 is from 90 wt. %:10 wt % to 10 wt. %:90 wt. %, preferably from 80 wt %:20 wt. % to 20 wt. %:80 wt. %, more preferably from 70 wt. %:30 wt. % to 30 wt. % to 70 wt. % to 50 wt. %,
(ii) (alkane) diols having 3 to 10 carbon atoms, preferably selected from the group consisting of 1,2-propylene glycol, 2-methylpropane-1,3-diol, 1,2-butylene glycol, 1,3-butanediol, 1,2-pentanediol, 1,3-pentanediol, 1,5-pentanediol, 2,4-pentanediol, 2-methyl-pentane-2,4-diol, 1,2-hexanediol, 1,6-hexanediol, 1,2-octanediol, dipropylene glycol, preferably 1,2-butylene glycol, 1,2-pentanediol and/or dipropylene glycol,
(iii) esters having 6 to 36 carbon atoms, preferably monoesters, diesters or triesters, preferably selected from the group consisting of diethyl phthalate, diethylhexyl 2,6-naphthalate, isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 3,5,5-trimethylhexyl 3,5,5-trimethylhexanoate, 2-ethylhexyl isononanoate, 2-ethylhexyl 3,5,5-trimethylhexanoate, 2-ethylhexyl 2-ethylhexanoate, 2-ethylhexyl palmitate, 2-ethylhexyl laurate, 2-hexyldecyl stearate, cetearyl ethylhexanoate, stearyl heptanoate, stearyl caprylate, 2-octyldodecyl palmitate, oleyl oleate, oleyl erucate, erucyl oleate, erucyl erucate, 2-ethylhexyl isostearate, isotridecyl isononanoate, 2-ethylhexyl cocoate, C₁₂₋₁₅-alkyl benzoates, cetyl palmitate, triethyl citrate, triacetin (triacetyl citrate), benzyl benzoate, benzyl acetate, vegetable oils (preferably olive oil, sunflower oil, soya oil, groundnut oil, rapeseed oil, almond oil, palm oil, coconut oil, palm kernel oil) and triglycerides, in particular glyceryl stearate, glyceryl triisononanoate, glyceryl laurate or triglycerides with identical or different C6 to C10 fatty acid radicals (so-called medium-chain triglycerides, in particular caprylic/capric triglyceride, like glyceryl tricaprylate, glyceryl tricaprate),
(iv) branched and unbranched alkyl or alkenyl alcohols, preferably selected from the group consisting of decanol, decenol, octanol, octenol, dodecanol, dodecenol, octadienol, decadienol, dodecadienol, oleyl alcohol, ricinoleyl alcohol, erucyl alcohol, stearyl alcohol, isostearyl alcohol, cetyl alcohol, lauryl alcohol, myristyl alcohol, arachidyl alcohol, linoleyl alcohol, linolenyl alcohol, hexyldecanol, octyldodecanol (in particular 2-octyl-1-dodecanol) and cetearyl alcohol and behenyl alcohol,
(v) branched and unbranched hydrocarbons and waxes, cyclic or linear silicone oils and dialkyl ethers having 6 to 24 carbon atoms, preferably selected from the group consisting of jojoba oil, isoeicosane, dicaprylyl ether, mineral oil, petrolatum, squalane, squalene, cyclomethicone, decamethylcyclopentasiloxane, undecamethylcyclotrisiloxane, polydimethylsiloxane and poly(methyl-phenyl siloxane,
(vi) solvents selected from the group consisting of acetone, methylpropyl ketone, dipropyl ketone, dimethyl sulfoxide, glycerine carbonate, propylene carbonate, butylene carbonate, glycerine formal, solketal, 2-ethyl hexanol, 2-butyl octanol, 2-hexyl decanol and 2-octyl dodecanol,
   is particularly preferred.

The above advantages particularly relate to carriers of group (i) as defined above.

Compositions according to the invention comprising such preferred carriers were found to have improved stability and advantageous effects regarding the solubility and/or (re-)crystallization properties of compounds of the general formula 1, in particular in (topical) cosmetic and pharmaceutical compositions, in particular compositions comprising water (preferably in an amount of 10 to 95 wt. %, more preferably 25 to 90 wt. %, even more preferably 40 to 90 wt. %, in each case based on the total weight of the composition or product), and in (topical) cosmetic and pharmaceutical compositions comprising water and an oil phase (e.g. O/W or W/O-emulsions).

By surprise, the inventors have found during their studies that specific surfactants may amplify the PAR-2 antagonistic and (consequently) the anti-itch properties of compounds of formulae 1 and of compositions comprising the same.

According to a preferred embodiment, the present invention also relates to a (concentrated) composition comprising or consisting of, based on the total weight of the composition,

0.1 to 50 wt. %, preferably 0.5 to 25 wt. %, and more preferably 1 to 10 wt. %, of compound(s) of the general formula 1 and/or salt(s) thereof, and
one or more carriers (preferably as described above), preferably one or more polyethyleneglycol esters, preferably according to formula 5, and/or one or more polyethyleneglycol ethers, preferably according to formula 6, preferably in a total amount of 50 wt. % or more, more preferably at least 70 wt. %, even more preferably 80 wt. % to 90 wt. % or most preferably in a total amount of at least 95 wt. %,
in particular
a) to improve solubility of compound(s) of formula 1 in cosmetic and/or pharmaceutical compositions and/or
b) to avoid re-crystallization of compound(s) of formula 1 in cosmetic and/or pharmaceutical formulations and/or
c) as a consequence of a) and/or b) to amplify the PAR-2 antagonistic activity as well as the skin soothing and/or anti-itch properties of compound(s) of formula 1.

The combination of compounds of formula 1 with polyethylene glycol esters of formula 5 and/or polyethyleneglycol ethers of formula 6 (cf. US20100150854A1) furtheron supports the skin soothing and, respectively, itch-reducing properties of compounds of general formula 1 because of the specific properties of polyethyleneglycol esters and polyethyleneglycol ethers to reduce, delay or prevent drying out of the skin, to regenerate the skin barrier function, and/or to moisturize the skin.

Such compositions are easy to handle and stable over a prolonged period of time (even at lower temperatures of about +10° C.), typically more than 3 months, preferably more than 6 months (at +5° C.), without compound(s) of the general formula crystallizing out of these compositions.

Compositions comprising one or more polyethylene esters and/or polyethylene ethers according to formula 5 and 6 (as described above) are readily further processable, in particular for (topical) cosmetic purposes.

Such a (concentrated) composition can be used for the preparation of cosmetic or pharmaceutical formulations or products, i.e. of further compositions according to the present invention (as described herein).

Also preferred is the use of one or more carriers selected from
one or more diols, preferably alkane diol(s), having 3 to 10 carbon atoms, preferably selected from the group consisting of 1,2-propylene glycol, 2-methylpropane-1,3-diol, 1,2-butylene glycol, 1,3-butanediol, 1,2-pentanediol, 1,3-pentanediol, 1,5-pentanediol, 2,4-pentanediol, 2-methyl-pentane-2,4-diol, 1,2-hexanediol, 1,6-hexanediol, 1,2-octanediol, 1,2-decanediol
and/or
cosmetically acceptable carriers selected from groups (i) and/or (ii) and/or (iii) and/or (iv) or mixtures thereof, said groups consisting of
(i) aliphatic esters having 6 to 36 carbon atoms, preferably monoesters, diesters or triesters, preferably selected from the group consisting of diethyl phthalate, diethylhexyl 2,6-naphthalate, isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 3,5,5-trimethylhexyl 3,5,5-trimethylhexanoate, 2-ethylhexyl isononanoate, 2-ethylhexyl 3,5,5-trimethylhexanoate, 2-ethylhexyl 2-ethylhexanoate, 2-ethylhexyl palmitate, 2-ethylhexyl laurate, 2-hexyldecyl stearate, cetearyl ethylhexanoate, stearyl isononanoate, palmityl isononanoate, cetrearyl isononanoate, palmityl 3,5,5-trimethylhexanoate, stearyl 3,5,5-trimethylhexanoate, cetearyl 3,5,5-trimethylhexanoate, stearyl heptanoate, stearyl caprylate, 2-octyldodecyl palmitate, oleyl oleate, oleyl erucate, erucyl oleate, erucyl erucate, 2-ethylhexyl isostearate, isotridecyl isononanoate, 2-ethylhexyl cocoate, C12-15-alkyl benzoates, cetyl palmitate, triethyl citrate, triacetin (triacetyl citrate), benzyl benzoate, benzyl acetate, vegetable oils (preferably olive oil, sunflower oil, soya oil, groundnut oil, rapeseed oil, almond oil, palm oil, coconut oil, palm kernel oil) and triglycerides, in particular glyceryl stearate, glyceryl triisononanoate, glyceryl laurate or triglycerides with identical or different C6 to C10 fatty acid radicals (so-called medium-chain triglycerides, in particular caprylic/capric triglyceride, like glyceryl tricaprylate, glyceryl tricaprate), and/or
(ii) branched and unbranched alkyl or alkenyl alkohols, preferably selected from the group consisting of decanol, decenol, octanol, octenol, dodecanol, dodecenol, octadienol, decadienol, dodecadienol, oleyl alcohol, ricinoleyl alcohol, erucyl alcohol, stearyl alcohol, isostearyl alcohol, cetyl alcohol, lauryl alcohol, myristyl alcohol, arachidyl alcohol, linoleyl alcohol, linolenyl alcohol, hexyldecanol, octyldodecanol (in particular 2-octyl-1-dodecanol) and cetearyl alcohol and behenyl alcohol, and/or
(iii) branched and unbranched hydrocarbons and waxes, cyclic or linear silicone oils and dialkyl ethers having 6 to 24 carbon atoms, preferably selected from the group consisting of jojoba oil, isoeicosane, dicaprylyl ether, mineral oil, petrolatum, squalane, squalene, cyclomethicone, decamethylcyclopentasiloxane, undecamethylcyclotrisiloxane, polydimethylsiloxane and poly(methyl-phenyl siloxane,
(iv) micellaneous other solvents like acetone, methylpropyl ketone, dipropyl ketone, dimethyl sulfoxide, glycerine carbonate, propylene carbonate, butylene carbonate, glycerine formal, solketal, 2-ethyl hexanol, 2-butyl octanol, 2-hexyl decanol or 2-octyl dodecanol.

Compositions comprising one or more cosmetically acceptable carriers as described above are easy to handle and stable over a long period of time, typically more than 3 months, preferably more than 6 months, without compounds of the general formula 1 crystallizing out of these compositions, which is particularly of importance in (cosmetic) compositions, in particular compositions comprising water in an amount of 10 wt. % or more, based on the total weight of the composition, or comprising a water and an oil phase, in particular emulsions, e.g. of the O/W- or W/O-type.

Such compositions are readily further processable, in particular for (topical) cosmetic purposes.

Compositions according to the present invention preferably comprise a total amount of 50 wt. % or more, more preferably at least 70 wt. %, even more preferably 80 wt. % to 90 wt. % or more, most preferably in a total amount of at least 95 wt. % or more and most preferred in a total amount of 95 to 99 wt. %, of carrier(s), preferably of the one or more (preferred) carriers as described above, in each case based on the total weight of the composition.

It was also found that compositions according to the present invention had improved storage stability (more than 3 months, generally more than 5 months) without compound(s) of the general formula 1 (re-)crystallizing from these compositions when
a total amount of 50 wt. % or more of the one or more preferred carriers as described above were present in case the amount of compound(s) of the general formula 1 was about 0.05 wt. % to 1.0 wt. %, and
a total amount of 50 wt. % or more (preferably up to 99 wt. %) of the one or more preferred carriers as described above were present in case the amount of compounds of the general formula 1 was higher than 1.0 wt. % to about 5.0 wt. %,
   in each case based on the total weight of the composition.

Particularly preferred is a composition according to the invention (as described above), additionally comprising one or more (further) substances (preferably suitable for cosmetic and/or dermatological applications) for preventing, reducing or alleviating itchy skin condition(s) and/or one or more skin irritation-reducing agents, in particular one or more substances selected from the group consisting of anti-inflammatory agents, physiological cooling agents and compounds that alleviate reddening, preferably wherein the one or more additional substances is/are selected from the group consisting of (the additional substances being substances other than compounds according to formula 1 or respective salts thereof):
(i) (further) anti-itch compounds,
(ii) steroidal anti-inflammatory substances of the corticosteroid type, in particular hydrocortisone, hydrocortisone derivatives such as hydrocortisone 17-butyrate, dexamethasone, dexamethasone phosphate, methylprednisolone or cortisone,
(iii) non-steroidal anti-inflammatory substances, in particular oxicams such as piroxicam or tenoxicam, salicylates such as aspirin, disalcid, solprin or fendosal, acetic acid derivatives such as diclofenac, fenclofenac, indomethacin, sulindac, tolmetin or clindanac, fenamates such as mefenamic, meclofenamic, flufenamic or niflumic, propionic acid derivatives such as ibuprofen, naproxen or benoxaprofen, pyrazoles such as phenylbutazone, oxyphenylbutazone, febrazone or azapropazone,
(iv) natural or naturally occurring anti-inflammatory substances or substances that alleviate reddening and/or itching, in particular extracts or fractions from camomile, Aloe vera, Commiphora species, Rubia species, willow, willow-herb, oats, calendula, arnica, St John's wort, honeysuckle, rosemary, Passiflora incarnate, witch hazel, ginger or Echinacea, or single active compounds thereof,
(v) alpha-bisabolol, apigenin, apigenin-7-glucoside, gingerols, shogaols, gingerdiols, dehydrogingerdiones, paradols, natural avenanthramides, non-natural avenanthramides, preferably dihydroavenanthramide D, boswellic acid, phytosterols, glycyrrhizin, glabridin and licochalcone A, preferably in the form of pure substances,
(vi) skin care agents, preferably skin moisture retention regulators or skin repair agents, preferably selected from the group consisting of sodium lactate, urea and derivatives, glycerol, propylene glycol, 1,2-pentanediol, 1,2-hexanediol and 1,2-octanediol, collagen, elastin or hyaluronic acid, diacyl adipates, petrolatum, urocanic acid, lecithin, allantoin, panthenol, phytantriol, lycopene, (pseudo-)ceramides (preferably Ceramide 2, hydroxypropyl bispalmitamide MEA, cetyloxypropyl glyceryl methoxypropyl myristamide, N-(1-hexadecanoyl)-4-hydroxy-L-proline (1-hexadecyl) ester, hydroxyethyl palmityl oxyhydroxypropyl palmitamide), glycosphingolipids, cholesterol, phytosterols, chitosan, chondroitin sulfate, lanolin, lanolin esters, amino acids, vitamin E and derivatives (preferably tocopherol, tocopheryl acetate), alpha-hydroxy acids (preferably citric acid, lactic acid, malic acid) and derivatives thereof, mono-, di- and oligosaccharides, preferably glucose, galactose, fructose, mannose, laevulose and lactose, polysugars, such as β-glucans, in particular 1,3-1,4-β-glucan from oats, alpha-hydroxy-fatty acids, triterpenic acids, such as betulic acid or ursolic acid, and algae extracts or single active compounds thereof,
(vii) physiological cooling agents, preferably selected from the group consisting of menthone glycerol acetal, menthyl lactate preferably l-menthyl lactate, in particular l-menthyl l-lactate), menthyl ethyl oxamate, substituted menthyl-3-carboxylic acid amides (e.g. menthyl-3-carboxylic acid N-ethylamide, N^{α}-(L-menthanecarbonyl)glycine ethyl ester, 2-isopropyl-N-2,3-trimethylbutanamide, substituted cyclohexanecarboxylic acid amides, 3-menthoxypropane-1,2-diol, 2-hydroxyethyl menthyl carbonate, 2-hydroxypropyl menthyl carbonate, N-acetylglycine menthyl ester, isopulegol, menthyl hydroxycarboxylic acid esters (e.g. menthyl 3-hydroxybutyrate), monomenthyl succinate, monomenthyl glutarate, 2-mercaptocyclodecanone, menthyl 2-pyrrolidin-5-onecarboxylate, 2,3-dihydroxy-p-menthane, 3,3,5-trimethylcyclohexanone glycerol ketal, 3-menthyl 3,6-di- and -trioxaalkanoates, 3-menthyl methoxyacetate and icilin, and
(viii) histamine receptor antagonists, serine protease inhibitors, TRPV1 antagonists, NK1 antagonists, cannabinoid receptor agonists and TRPV3 antagonists, in particular such as exemplary described above.

Particularly preferred is a composition according to the invention, wherein the total amount of compound(s) of formula 1 and/or respective salt(s) thereof is sufficient to a) prevent, reduce or alleviate one or more itchy skin conditions, and/or to b) provide a PAR-2 antagonistic effect.

However, preferred compositions according to the invention may also comprise, as described above, for example one or more TRPV1 antagonists, TRPV3 antagonists, serine protease inhibitors, histamine receptor antagonists and/or cannabinoid receptor agonists, further amplifying the efficacy of compound(s) of general formula 1 or respective salt(s) thereof via inhibition of (alternative) itch generating biological pathways.

Preferably, the total amount of one or more (further) substances (preferably suitable for cosmetic and/or dermatological applications) for preventing, reducing or alleviating itchy skin condition(s) and/or one or more skin irritation-reducing agents, in particular one or more substances selected from the group consisting of anti-inflammatory agents, physiological cooling agents and compounds that alleviate reddening, in particular of substances of groups (i) to (viii) as described above, in the composition of the present invention is preferably 0.0001 to 20 wt. %, particularly preferably 0.0001 to 10 wt. %, in particular 0.001 to 5 wt. %, based on the total weight of the composition.

Also preferred is a composition (as described above) additionally comprising one or more (further) substances selected from the groups consisting of:
extracts or fractions from camomile, Aloe vera, oats, calendula, arnica, honeysuckle, rosemary, witch hazel, ginger or Echinacea,
alpha-bisabolol, gingerols, shogaols, gingerdiols, dehydrogingerdiones, paradols, natural avenanthramides, non-natural avenanthramides, preferably dihydroavenanthramide D, boswellic acid, phytosterols, glycyrrhizin, and licochalcone A,
urea, hyaluronic acid, allantoin, panthenol, lanolin, alpha-hydroxy acids (preferably citric acid, lactic acid), vitamin E and derivatives thereof (preferably tocopherol, tocopheryl acetate).

In another preferred embodiment, a composition according to the present invention additionally comprises one or more fragrance materials. Suitable fragrance materials are mentioned in S. Arctander, Perfume and Flavor Chemicals, Vol. I and II, Montclair, N. J., 1969, self-published or H. Surburg and J. Panten, Common Fragrance and Flavor Materials, 5th. Ed., Wiley-VCH, Weinheim 2006, particularly those explicitly mentioned in US 2008/0070825.

Compositions according to the present invention advantageously comprise a total amount of 0.1 to 5 wt. %, preferably 0.2 to 4 wt. %, more preferably 0.25 to 3 wt. %, even more preferably 0.3-2.5 wt. %, of the one or more (preferred) fragrance materials, in each case based on the total weight of the composition or product.

More preferably the fragrance materials are selected from (here in some cases the normal industrial product names and registered trademarks of various firms are given):

alpha-amyl cinnamic aldehyde, alpha-hexyl cinnamic aldehyde, 2-phenoxyethylisobutyrate (Phenirat), methyl dihydrojasmonate [preferably with a content of cis-isomers of >60 by weight (Hedione, Hedione HC)], 4,6,6,7,8,8-hexamethyl-1,3,4,6,7,8-hexahydrocyclopenta[g]benzopyran (Galaxolide), benzylsalicylate, 2-methyl-3-(4-tert-butyl-phenyl)propanal (Lilial), 4,7-methano-3a,4,5,6,7,7a-hexahydro-5-indenyl acetate and/or 4,7-methano-3a,4,5,6,7,7a-hexahydro-6-indenyl acetate (Herbaflorat), styrallyl acetate(1-phenylethyl acetate), octahydro-2,3,8,8-tetramethyl-2-acetonaphthone and/or 2-acetyl-1,2,3,4,6,7,8-octahydro-2,3,8,8-tetramethylnaphthaline (Iso E Super), hexylsalicylate, 4-tert.-butylcyclohexyl acetate (Oryclon), 2-tert.-butylcyclohexyl acetate (Agrumex HC), alpha-ionone (4-(2,2,6-trimethyl-2-cyclohexen-1-yl)-3-buten-2-one), 4-(4-hydroxy-4-methylpentyl)-3-cyclohexene carboxaldehyde (Lyral), (E)- and/or (Z)-3-methylcyclopentadec-5-enone (Muscenone), 15-pentadec-11-enolide and/or 15-pentadec-12-enolide (Globalide), 15-cyclopentadecanolide (Macrolide), 1-(5,6,7,8-tetrahydro-3,5,5,6,8,8-hexamethyl-2-naphthalenyl)ethanone (Tonalide), ethylene brassylate, 2-ethyl-4-(2,2,3-trimethyl-3-cyclopenten-1-yl)-2-buten-1-ol (Sandranol), alpha-Santalol, 2,2-dimethyl-3-(3-methylphenyl)-propanol (Majantol), allyl heptanoate, 4-methylacetophenone, (4aR,5R,7aS,9R)-octahydro-2,2,5,8,8,9a-hexamethyl-4H-4a,9-methanoazuleno(5,6-d)-1,3-dioxol) (Ambrocenide), Timberol (1-(2,2,6-trimethylcyclohexyl)hexan-3-ol), benzylacetone, methyl cinnamate, 3a,6,6,9a-tetramethyldodecahydronaphtho[2,1-b]furan (Ambroxid).

The total amount of fragrance materials selected from the above group, preferably having a boiling point of 250° C. or greater at 1013 mbar, preferably is at least 10 wt. %, more preferably at least 20 wt. %, based on the total amount of fragrance materials present in a composition according to the present invention.

When bisabolol is used in the context of the present invention it can be of natural or synthetic origin, and is preferably “alpha-bisabolol”. Preferably, the bisabolol used is synthetically prepared or natural (−)-alpha-bisabolol and/or synthetic mixed-isomer alpha-bisabolol. If natural (−)-alpha-bisabolol is used, this can also be employed as a constituent of an essential oil or of a plant extract or of a fraction thereof, for example as a constituent of (fractions of) oil or extracts of camomile or of Vanillosmopsis (in particular Vanillosmopsis erythropappa or Vanillosmopsis arborea). Synthetic alpha-bisabolol is obtainable, for example, under the name “Dragosantol” from Symrise.

In case ginger extract is used in the context of the present invention, preferably extracts of the fresh or dried ginger root are used which are prepared by extraction with methanol, ethanol, iso-propanol, acetone, ethyl acetate, carbon dioxide (CO₂), hexane, methylene chloride, chloroform or other solvents or solvent mixtures of comparable polarity. The extracts are characterized by the presence of active skin irritation-reducing amounts of constituents such as e.g. gingerols, shogaols, gingerdiols, dehydrogingerdiones and/or paradols.

A composition according to the present invention can be further processed by encapsulation with a solid shell material, which is preferably chosen from starches, degraded or chemically or physically modified starches (in particular dextrins and maltodextrins), gelatines, wax materials, liposomes, gum arabic, agar-agar, ghatti gum, gellan gum, modified and non-modified celluloses, pullulan, curdlan, carrageenans, algic acid, alginates, pectin, inulin, xanthan gum and mixtures of two or more of the substances mentioned.

The cosmetic or pharmaceutical compositions according to the invention can be produced by conventional processes known per se, such that compound(s) of the general formula 1 and/or respective salt(s) thereof are incorporated into (topical) cosmetic or pharmaceutical compositions which (in addition to the aforementioned effects) can also be used for the treatment, care and/or cleansing of the skin or hair.

Preferred fields of use for compositions according to the invention are (preferably topical) cosmetic, dermatological or therapeutic products which serve for cosmetic or dermatological light protection, for treatment, care and cleansing of the skin and/or hair or as a make-up product in decorative cosmetics. Such products can accordingly be present e.g. as a cleansing composition, such as e.g. soap, syndet, liquid washing, shower and bath preparation, skin care composition, such as e.g. emulsion (as a solution, dispersion, suspension; cream, lotion or milk of the W/O, O/W or multiple emulsion, PIT emulsion, emulsion foam, micro- or nanoemulsion, Pickering emulsion type, depending on the preparation process and constituents), ointment, paste, gel (including hydro-, hydrodispersion-, oleogel), alcoholic or aqueous/alcoholic solution, oil, toner, balsam, serum, powder (e.g. face powder, body powder), soaking liquid for wipes, Eau de Toilette, Eau de Cologne, perfume, wax, including the presentation form as a mask, mousse, stick, pencil, roll-on, (pump) spray, aerosol (foaming, non-foaming or after-foaming), skin care composition (as described above) as a foot care composition (including keratolytics, deodorant), as an insect repellent composition, as a sunscreen composition, as a self-tanning composition and/or aftersun preparation, skin care composition as a shaving composition or after-shave, as a hair-removing composition, as a hair care composition, such as e.g. shampoo (including shampoo for normal hair, for greasy hair, for dry, stressed (damaged) hair, 2-in-1 shampoo, anti-dandruff shampoo, baby shampoo, shampoo for a dry scalp, shampoo concentrate), conditioner, hair treatment cure, hair tonic, hair lotion, hair rinse, styling cream, pomade, permanent wave and fixing compositions, hair smoothing composition (straightening composition, relaxer), hair setting composition, styling aid (e.g. gel or wax); blonding composition, hair colouring composition, such as e.g. temporary, directly absorbed, semi-permanent hair colouring composition, permanent hair colouring composition, skin care composition as a decorative body care composition, such as e.g. nail care composition (nail varnish and nail varnish remover), decorative cosmetic (e.g. powder, eye shadow, kajal pencil, lipstick, mascara), make-up, make-up remover, skin care composition as a deodorant and/or antiperspirant.

Preferred products or, respectively, compositions according to the present inventions are selected from the group of pharmaceutical and/or cosmetic products for treatment, protecting, care and cleansing of the skin and/or hair or as a make-up product, preferably as a leave-on product, more preferably in the form or selected from the product group consisting of alcoholic or aqueous/alcoholic solution, dispersion, suspension, emulsion (preferably cream, lotion or milk of the W/O, O/W or multiple emulsion, PIT emulsion, emulsion foam, micro-, nanoemulsion, Pickering emulsion type), ointment, paste, gel (preferably hydro-, hydrodispersion-, oleogel), balm, serum, powder, wipe, Eau de Toilette, Eau de Cologne, perfume, stick, roll-on, (pump) spray, aerosol, leave-on skin care composition (preferably face-care composition), leave-on insect repellent composition, sunscreen composition, skin-lightening composition, self-tanning composition, aftersun preparation, shaving or after-shave composition, hair-removing composition, hair care composition, preferably conditioner, hair lotion, hair tonic, styling cream, pomade, styling aid (preferably gel or wax), permanent wave and fixing compositions, hair smoothing composition (straightening composition, relaxer), hair setting composition, blonding composition, hair colouring composition, such as e.g. temporary, directly absorbed, semi-permanent hair colouring composition, permanent hair colouring composition, decorative cosmetic composition (preferably face powder, eye shadow, kajal pencil, lipstick), deodorant and/or antiperspirant composition.

A further aspect of the present invention relates to a cosmetic, preferably non-therapeutic, or therapeutic method
a) for preventing, reducing or alleviating one or more itchy conditions, and/or
b) for providing a PAR-2 antagonistic effect,
   comprising the following steps:
   providing
      one or more compounds selected from the group consisting of compounds of formula 1, 2, 3 and 4,

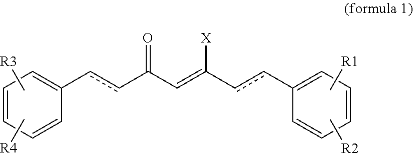

with R1, R2, R3 and R4 independently from each other meaning —OH, —OR or —OCOR, with R meaning an alkyl group comprising 1-12 carbon atoms,
with [symbol] meaning an optional additional bond, wherein, according to a preferred aspect of the invention, only one or none of the dotted lines ( [symbol] ) means an additional bond, and
with X meaning —H or —OH,

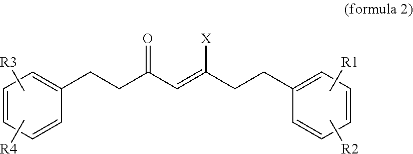

with R1, R2, R3 and R4 independently from each other meaning —OH, —OR or —OCOR, with R meaning an alkyl group comprising 1-12 carbon atoms, and
with X meaning —H or —OH,

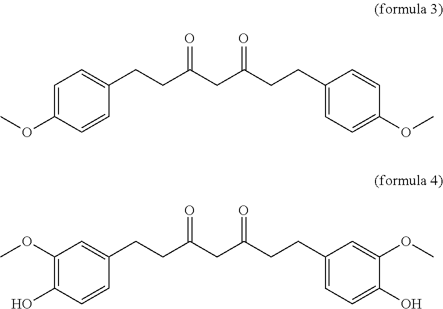

and/or one or more respective salts thereof, preferably one or more cosmetically and/or pharmaceutically acceptable salts thereof, in particular Na⁺, K⁺, NH₄⁺, Mg²⁺ or Ca²⁺ salts,
or
a composition according to the invention (as described above),
applicating the compound(s) and/or salt(s) or, respectively, the composition to, for prophylaxis, non-itchy skin or to, for treatment, itchy skin in an effective amount to
a) prevent, reduce or alleviate one or more itchy skin conditions, and/or
b) for providing a PAR-2 antagonistic effect.

Again, the above explanations regarding preferred compounds and/or salts to be used in connection with the present invention or preferred effects/uses thereof also apply to the above described method.

Particularly preferred is a method, wherein the applicated compound(s) and/or salt(s) or, respectively, the composition remains for at least 5 minutes, more preferably for at least 10 minutes, on said skin (“leave-on product”).

Compositions and, respectively, products, in particular (topical) cosmetic products, according to the present invention can advantageously additionally comprise suitable auxiliary substances and additives, such as, for example:

preservatives, in particular those described in US 2006/0089413, antimicrobial agents, such as e.g. antibacterial agents or agents to treat yeast and mold, in particular those described in WO 2005/123101, antiacne and sebum reducing agents, in particular those described in WO 2008/046791, compounds against ageing of the skin, in particular those described in WO 2005/123101, antidandruff agents, in particular those described in WO 2008/046795, antiirritants (antiinflammatory agents, irritation-preventing agents, irritation-inhibiting agents), in particular those described in WO 2007/042472 and US 2006/0089413, antioxidants, in particular those described in WO 2005/123101, carrier materials, in particular those described in WO 2005/123101, chelating agents, in particular those described in WO 2005/123101, deodorizing agents and antiperspirants, in particular those described in WO 2005/123101, moisture regulators (moisture-donating agents, moisturizing substance, moisture-retaining substances), in particular those described in WO 2005/123101, osmolytes, in particular those described in WO 2005/123101, compatible solutes, in particular those described in WO 01/76572 and WO 02/15868, proteins and protein hydrolysates, in particular those described in WO 2005/123101 and WO 2008/46676, skin-lightening agents, in particular those described in WO 2007/110415, skin-tanning agents, in particular those described in WO 2006/045760, cooling agents, in particular those described in WO 2005/123101, skin-cooling agents, in particular those described in WO 2005/123101, skin warming agents, in particular those described in WO 2005/123101, UV-absorbing agents, in particular those described in WO 2005/123101, UV filters, in particular those described in WO 2005/123101, benzylidene-beta-dicarbonyl compounds in accordance with WO 2005/107692 and alpha-benzoyl-cinnamic acid nitriles in accordance with WO 2006/015954, insect repellents, in particular those described in WO 2005/123101, plant parts, plant extracts, in particular those described in WO 2005/123101, vitamins, in particular those described in WO 2005/123101, emulsifiers, in particular those described in WO 2005/123101, gelling agents, in particular those described in WO 2005/123101, oils in particular those described in WO 2005/123101, waxes in particular those described in WO 2005/123101, fats in particular those described in WO 2005/123101, phospholipids, in particular those described in WO 2005/123101, saturated fatty acids and mono- or polyunsaturated fatty acids and α-hydroxy acids and polyhydroxy-fatty acids and esters of saturated and/or unsaturated branched and/or unbranched alkane carboxylic acids, in particular those described in WO 2005/123101, surface-active substances (surfactants) in particular those described in WO 2005/123101, skin repair agents comprising cholesterol and/or fatty acids and/or ceramides and/or pseudoceramides, in particular those described in WO 2006/053912, dyestuffs and colorants and pigments, in particular those described in WO 2005/123101, aroma chemicals and flavors and fragrances, in particular those described in S. Arctander, Perfume and Flavor Chemicals, private publishing house, Montclair, N. J., 1969 and Surburg, Panten, Common Fragrance and Flavor Materials, 5th Edition, Wiley-VCH, Weinheim 2006, preferably those explicitly mentioned in US 2008/0070825, alcohols and polyols, in particular those described in WO 2005/123101, organic solvents, in particular those described in WO 2005/123101, silicones and silicone oils and silicone derivatives in particular those described in WO 2008/046676, virucides, abrasives, anti-cellulite agents, astringents, antiseptic agents, antistatics, binders, buffers, cell stimulants, cleansing agents, care agents, depilatory agents, softeners, enzymes, essential oils, in particular those described in US 2008/0070825, fibres, film-forming agents, fixatives, foam-forming agents, foam stabilizers, substances for preventing foaming, foam boosters, gel-forming agents, hair growth activators, hair growth inhibitors, hair care agents, hair-setting agents, hair-straightening agents, hair-smoothening, bleaching agents, strengthening agents, stain-removing agents, optically brightening agents, impregnating agents, dirt-repellent agents, friction-reducing agents, lubricants, opacifying agents, plasticizing agents, covering agents, polish, gloss agents, polymers in particular those described in WO 2008/046676, powders, peptides, mono-, di- and oligosaccharides, re-oiling agents, abrading agents, skin-soothing agents, skin-cleansing agents, skin care agents, skin-healing agents, skin-protecting agents, skin-softening agents, skin-smoothing agents, nourishing agents, skin-warming agents, stabilizers, detergents, fabric conditioning agents, suspending agents, thickeners, yeast extracts, algae or microalgae extracts, animal extracts, liquefiers, color-protecting agents, and electrolytes.

The (in particular topical) cosmetic or pharmaceutical products according to the invention can comprise cosmetic auxiliary substances and additives such as are conventionally used in such formulations, e.g. sunscreen agents, preservatives, bactericides, fungicides, virucides, cooling active compounds, insect repellents (e.g. DEET, IR 3225), plant extracts, plant parts, antiinflammatory active compounds, substances which accelerate wound healing (e.g. chitin or chitosan and derivatives thereof), film-forming substances (e.g. polyvinylpyrrolidones or chitosan or derivatives thereof), antioxidants, vitamins, 2-hydroxycarboxylic acids (e.g. citric acid, malic acid, L-, D- or dl-lactic acid), skin-colouring agents (e.g. walnut extracts or dihydroxyacetone), active compounds for promoting hair growth or inhibiting hair growth, skin care compositions (e.g. cholesterol, ceramides, pseuodceramides), softening, moisturizing and/or humectant substances, fats, oils, saturated fatty acids, mono- or polyunsaturated fatty acids, α-hydroxy acids, polyhydroxy-fatty acids or derivatives thereof, waxes or other conventional constituents of a cosmetic or dermatological formulation, such as alcohols, polyols, polymers, foam stabilizers, electrolytes, organic solvents, silicone derivatives of chelating agents (e.g. ethylenediaminetetraacetic acid and derivatives), antidandruff active compounds (e.g. climbazole, ketoconazole, piroctonoleamine, zinc pyrithione), hair care agents, perfumes, substances for preventing foaming, dyestuffs, pigments which have a colouring action, thickening agents (advantageously silicon dioxide, aluminium silicates, such as e.g. bentonites, polysaccharides or derivatives thereof, e.g. hyaluronic acid, guar bean flour, xanthan gum, hydroxypropylmethylcellulose or allulose derivatives, particularly advantageously polyacrylates, such as e.g. Carbopols or polyurethanes), surface-active substances and emulsifiers.

Auxiliary substances and additives (excluding water) can generally be included in products according to the present invention in quantities of 1 to 95 wt. %, preferably 5 to 70 wt. %, more preferably 5 to 50 wt. %, in each case based on the total weight of the product. The amounts of cosmetic or dermatological auxiliary agents and additives and perfume to be used in each case can easily be determined by the person skilled in the art by simple trials, depending on the nature of the particular product.

According to one aspect of the invention, the products or, respectively, compositions according to the present invention preferably contain water in a quantity of up to 98 wt. %, preferably 10 to 95 wt. %, more preferably 25 to 90 wt. %, even more preferably 40 to 90 wt. %, in each case based on the total weight of the product.

The formulations according to the invention can also comprise antioxidants, it being possible for all the antioxidants which are suitable or usual for cosmetic and/or dermatological uses to be used. The antioxidants are advantageously chosen from the group consisting of:

amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, liponic acid and derivatives thereof (e.g. dihydroliponic acid), aurothioglucose, propylthiouracil and other thiols (e.g. thioredoxin, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), (metal) chelators, e.g. α-hydroxy-fatty acids, palmitic acid, phytic acid, lactoferrin, α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate, ascorbyl glycosides, such as e.g. 6-O-acyl-2-O-α-D-g lucopyranosyl-L-ascorbic acid, 6-O-acyl-2-O-β-D-g lucopyranosyl-L-ascorbic acid, 2-O-α-D-glucopyranosyl-L-ascorbic acid or 2-O-β-D-glucopyranosyl-L-ascorbic acid), tocopherols and derivatives thereof (e.g. vitamin E acetate), vitamin A and derivatives thereof (vitamin A palmitate) as well as coniferylbenzoate of benzoin resin, rutic acid and derivatives thereof, α-glucosylrutin, quercetin and derivatives thereof, rosemary acid, carnosol, carnosol acid, resveratrol, caffeic acid and derivatives thereof, sinapic acid and derivatives thereof, ferulic acid and derivatives thereof, furfurylideneglucitol, butylhydroxytoluene, butylhydroxyanisole, nordihydroguaiac resin acid, nordihydroguaiaretic acid, trihydroxybutyrophenone, uric acid and derivatives thereof, mannose and derivatives thereof, superoxide dismutase, zinc and derivatives thereof (e.g. ZnO, ZnSO₄), selenium and derivatives thereof (e.g. selenium methionine), stilbenes and derivatives thereof (e.g. stilbene oxide, trans-stilbene oxide) and derivatives (salts, esters, ethers, sugars, nucleotides, nucleosides, peptides and lipids) of these active compounds mentioned or antioxidatively active extracts or fractions from plants, such as e.g. green tea, rooibos, honeybush, grape, rosemary, sage, Melissa, thyme, lavender, olive, oats, cocoa, ginkgo, ginseng, liquorice, honeysuckle, Sophora, Pueraria, Pinus, Citrus, Phyllanthus emblica or St. John's wort.

The amount of antioxidants (preferably the amount of antioxidants of the above list) in the formulations according to the invention is preferably 0.01 to 20 wt. %, particularly preferably 0.05 to 10 wt. %, in particular 0.2-5 wt. %, based on the total weight of the formulation.

The formulations and products (compositions) according to the present invention can also comprise physiological warming (heating) agents, which in some cases are TRPV1 agonists and thus are substances which may cause skin irritations. Such physiological warming agents preferably are selected from the group consisting of vanillyl alcohol n-butyl ether, vanillyl alcohol n-propyl ether, vanillyl alcohol isopropyl ether, vanillyl alcohol isobutyl ether, vanillyl alcohol n-amino ether, vanillyl alcohol isoamyl ether, vanillyl alcohol n-hexyl ether, vanillyl alcohol methyl ether, vanillyl alcohol ethyl ether, gingerol, shogaol, zingerone, capsaicin, dihydrocapsaicin, nordihydrocapsaicin, homocapsaicin, homodihydrocapsaicin, iso-propyl alcohol, iso-amylalcohol, benzyl alcohol, eugenol, cinnamon oil, cinnamic aldehyde, and mixtures thereof.

The formulations according to the invention may advantageously comprise at least one UVA filter and/or at least one UVB filter and/or at least one inorganic pigment. In this context, the formulations can be in various forms such as are conventionally employed e.g. for sunscreen formulations for protecting the skin and hair against ultraviolet radiation. They can thus form e.g. a solution, an emulsion of the water-in-oil (W/O) type or of the oil-in-water (O/W) type or a multiple emulsion, for example of the water-in-oil-in-water (W/O/W) type, a gel, a hydrodispersion, a solid stick or also an aerosol. In this context, the total amount of UV-filter substances is from 0.01 wt. % to 40 wt. %, preferably 0.1 to 10 wt. %, in particular 1.0 to 5.0 wt. %, based on the total weight of the formulations.

Advantageous UV filters are e.g.:

p-aminobenzoic acid, p-aminobenzoic acid ethyl ester (25 mol) ethoxylated, p-dimethylaminobenzoic acid 2-ethylhexyl ester, p-aminobenzoic acid ethyl ester (2 mol) N-propoxylated, p-aminobenzoic acid glycerol ester, salicylic acid homomenthyl ester (homosalate) (Neo Heliopan® 1-IMS), salicylic acid 2-ethylhexyl ester (Neo Heliopan® OS), triethanolamine salicylate, 4-isopropylbenzyl salicylate, anthranilic acid menthyl ester (Neo Heliopan® MA), diisopropylcinnamic acid ethyl ester, p-methoxycinnamic acid 2-ethylhexyl ester (Neo Heliopan® AV), diisopropylcinnamic acid methyl ester, p-methoxycinnamic acid isoamyl ester (Neo Heliopan® E 1000), p-methoxycinnamic acid diethanolamine salt, p-methoxycinnamic acid isopropyl ester, 2-ethylhexyl 2-cyano-3,3-diphenylacrylate (Neo Heliopan® 303), ethyl 2-cyano-3,3′-diphenylacrylate, 2-phenylbenzimidazolesulfonic acid and salts (Neo Heliopan® Hydro), 3-(4′-trimethylammonium)-benzylidene-bornan-2-one methyl-sulfate, terephthalylidene-dibornanesulfonic acid and salts (Mexoryl® SX), 4-t-butyl-4′-methoxy-dibenzoylmethane (avobenzone)/(Neo Heliopan® 357), β-Imidazole-4(5)-acrylic acid (urocanic acid), 2-hydroxy-4-methoxybenzophenone (Neo Heliopan® BB), 2-hydroxy-4-methoxybenzophenone-5-sulfonic acid, dihydroxy-4-methoxybenzophenone, 2,4-dihydroxybenzophenone, tetrahydroxybenzophenone, 2,2′-dihydroxy-4,4′-dimethoxybenzophenone, 2-hydroxy-4-n-octoxybenzophenone, 2-hydroxy-4-methoxy-4′-methylbenzophenone, 3-(4′-sulfo)benzylidene-bornan-2-one and salts, 3-(4′-methylbenzylidene)-d,l-camphor (Neo Heliopan® MBC), 3-benzylidene-d,l-camphor, 4-isopropyldibenzoylmethane, 2,4,6-trianilino-(p-carbo-2′-ethylhexyl-1′-oxy)-1,3,5-triazine, phenylene-bis-benzimidazyl-tetrasulfonic acid disodium salt (Neo Heliopan® AP), 2,2′-(1,4-phenylene)-bis-(1H-benzimidazole-4,6-disulfonic acid), monosodium salt, N-[(2 and 4)-[2-(oxoborn-3-ylidene)methyl]benzyl]-acrylamide polymer, phenol, -(2H-benzotriazol-2-yl)-4-methyl-6-(2-methyl-3(1,3,3,3-tetramethyl-1-(trimethylsilyl)-oxy)-disiloxyanyl)-propyl), (Mexoryl® XL), 4,4′-[(6-[4-(1,1-dimethyl)-aminocarbonylyphenylamino]-1,3,5-triazine-2,4-diyl)diimino]-bis-(benzoic acid 2-ethylhexyl ester) (Uvasorb® HEB), 2,2′-methylene-bis-(6-(2H-benzotriazol-2-yl)-4-(1,1,3,3-tetramethylbutyl)-phenol), (Tinosorb® M), 2,4-bis-[4-(2-ethylhexyloxy)-2-hydroxyphenyl]-1,3,5-triazine, benzylidene malonate-polysiloxane (Parsol® SLX), glyceryl ethylhexanoate dimethoxycinnamate, disodium 2,2′-dihydroxy-4,4′-dimethoxy-5,5′-d isulfo-benzophenone, dipropylene glycol salicylate, sodium hydroxymethoxybenzophenone-sulfonate, 4,4′,4-(1,3,5-triazine-2,4,6-triyltriimino)-tris-benzoic acid tris(2-ethylhexyl ester) (Uvinul® T150), 2,4-bis-[{(4-(2-ethyl-hexyloxy)-2-hydroxy}-phenyl]-6-(4-methoxyphenyl)-1,3,5-triazine, (Tinosorb® S), 2,4-bis-[{(4-(3-sulfonato)-2-hydroxy-propyloxy)-2-hydroxy}-phenyl]-6-(4-methoxyphenyl)-1,3,5-triazine sodium salt, 2,4-bis-[{(3-(2-propyloxy)-2-hydroxy-propyloxy)-2-hydroxy}-phenyl]-6-(4-methoxy-phenyl)-1,3,5-triazine, 2,4-bis-[{4-(2-ethyl-hexyloxy)-2-hydroxy}-phenyl]-6-[4-(2-methoxyethyl-carbonyl)-phenylamino]-1,3,5-triazine, 2,4-bis-[{4-(3-(2-propyloxy)-2-hydroxy-propyloxy)-2-hydroxy}-phenyl]-6-[4-(2-ethylcarboxyl)-phenylamino]-1,3,5-triazine, 2,4-bis-[{4-(2-ethyl-hexyloxy)-2-hydroxy}-phenyl]-6-(1-methyl-pyrrol-2-yl)-1,3,5-triazine, 2,4-bis-[{4-tris-(trimethylsiloxy-silylpropyloxy)-2-hydroxy}-phenyl]-6-(4-methoxyphenyl)-1,3,5-triazine, 2,4-bis-[{4-(2″-methylpropenyloxy)-2-hydroxy}-phenyl]-6-(4-methoxyphenyl)-1,3,5-triazine, 2,4-bis-[{4-(1′,1′,1′,3′,5′,5′,5′-heptamethylsiloxy-2″-methyl-propyloxy)-2-hydroxy}-phenyl]-6-(4-methoxyphenyl)-1,3,5-triazine, 2-(4-diethylamino-2-hydroxybenzoyl)-benzoic acid hexyl ester (Uvinul® A Plus) and indanylidene compounds according to DE 100 55 940 (=WO 02/38537).

In this context, UV absorbers which are particularly suitable for combination are p-aminobenzoic acid, 3-(4′-trimethylammonium)-benzylidene-bornan-2-one methyl-sulfate, salicylic acid homomenthyl ester (Neo Heliopan® HMS), 2-hydroxy-4-methoxy-benzophenone (Neo Heliopan® BB), 2-phenylbenzimidazolesulfonic acid (Neo Heliopan® Hydro), terephthalylidene-dibornanesulfonic acid and salts (Mexoryl® SX), 4-tert-butyl-4′-methoxydibenzoylmethane (Neo Heliopan® 357), 3-(4′-sulfo)benzylidene-bornan-2-one and salts, 2-ethylhexyl 2-cyano-3,3-diphenylacrylate (Neo Heliopan® 303), N-[(2 and 4)-[2-(oxoborn-3-ylidene)methyl]benzyl]-acrylamide polymer, p-methoxycinnamic acid 2-ethylhexyl ester (Neo Heliopan® AV), p-aminobenzoic acid ethyl ester (25 mol) ethoxylated, p-methoxycinnamic acid isoamyl ester (Neo Heliopan® E1000), 2,4,6-trianilino-(p-carbo-2′-ethylhexyl-1′-oxy)-1,3,5-triazine (Uvinul® T150), phenol, 2-(2H-benzotriazol-2-yl)-4-methyl-6-(2-methyl-3(1,3,3,3-tetramethyl-1-(trimethylsilyl)-oxy)-disiloxyanyl)-propyl), (Mexoryl® XL), 4,4′-[(6-[4-(1,1-dimethyl)-aminocarbonyl)-phenylamino]-1,3,5-triazine-2,4-diyl)-diimino]-bis-(benzoic acid 2-ethylhexyl ester), (UvasorbHEB), 3-(4′-methylbenzylidene)-d,l-camphor (Neo Heliopan® MBC), 3-benzylidenecamphor, salicylic acid 2-ethylhexyl ester (Neo Heliopan® OS), 4-dimethylaminobenzoic acid 2-ethylhexyl ester (Padimate 0), hydroxy-4-methoxy-benzophenone-5-sulfonic acid and Na salt, 2,2′-methylene-bis-(6-(2H-benzotriazol-2-yl)-4-(1,1,3,3-tetramethylbutyl)-phenol), (Tinosorb® M), phenylene-bis-benzimidazyl-tetrasulfonic acid disodium salt (Neo Heliopan® AP), 2,4-bis-[{(4-(2-ethyl-hexyloxy)-2-hydroxy}-phenyl]-6-(4-methoxyphenyl)-1,3,5-triazine, (Tinosorb® S), benzylidene malonate-polysiloxane (Parsol® SLX), menthyl anthranilate (Neo Heliopan® MA), 2-(4-diethylamino-2-hydroxybenzoyl)-benzoic acid hexyl ester (Uvinul® A Plus) and indanylidene compounds according to DE 100 55 940 (=WO 02/38537).

Advantageous inorganic sunscreen pigments are finely disperse metal oxides and metal salts, for example titanium dioxides, zinc oxide (ZnO), iron oxides (e.g. Fe₂O₃), aluminium oxide (Al₂O₃); cerium oxides (e.g. Ce₂O₃), manganese oxides (e.g. MnO), zirconium oxide (ZrO₂), silicon oxide (SiO₂), mixed oxides of the corresponding metals and mixtures of such oxides, barium sulfate and zinc stearate. They are particularly preferably pigments based on TiO₂ or zinc oxide. In preferred embodiments, the particles have an average diameter of less than 100 nm, preferably between 5 and 50 nm and particularly preferably between 15 and 30 nm. They can have a spherical shape, but those particles which have an ellipsoid shape or a shape which deviates otherwise from the spherical can also be employed. The pigments can also be in a form treated on the surface, i.e. hydrophilized or hydrophobized. Typical examples are coated titanium dioxides, such as e.g. titanium dioxide T 805 (Degussa) or Eusolex® T2000 (Merck), or coated zinc oxide, such as e.g. Zinc Oxide NDM. In this context, possible hydrophobic coating agents are, above all, silicones, and in this case specifically trialkoxyoctysilanes or simethicone. So-called micro- or nanopigments are preferably employed in sunscreen compositions. Zinc micro- or nanopigments are preferably employed.

The total amount of inorganic pigments, in particular hydrophobic inorganic micropigments, in the finished cosmetic or dermatological formulations is advantageously in the range of from 0.1 to 30 wt. %, preferably 0.1 to 10.0, in particular 0.5 to 6.0 wt. %, based on the total weight of the formulations.

Cosmetic formulations according to the invention which comprise a composition according to the invention having a skin irritation-reducing action can also comprise active compounds and active compound combinations against ageing of the skin and wrinkles. According to the invention, all the active compounds against ageing of the skin and wrinkles which are suitable or usual for cosmetic and/or dermatological uses can be used here. Advantageous active compounds against ageing of the skin and wrinkles in this respect are soya protein or protein hydrolysates, soya isoflavones, hydrolyzed rice protein, hydrolysed hazelnut protein, oligopeptides from hydrolysed Hibiscus esculentus extract, wheat protein, β-glucans, e.g. from oats, and derivatives thereof, glycoproteins, ursolic acid and its salts, betulin, betulic acid and its salts, retinol, retinol palmitate, propyl gallate, precocenes, 6-hydroxy-7-methoxy-2,2-dimethyl-1(2H)-benzopyran, 3,4-dihydro-6-hydroxy-7-methoxy-2,2-dimethyl-1(2H)-benzopyran, creatine or other synthetic or natural active compounds against ageing of the skin and wrinkles, it being possible for the latter also to be used in the form of an extract from plants, such as e.g. green tea, Rubus fruticosus, Sanguisorba officinalis, Centella asiatica, Ribes nigrum, Passiflora incarnate, Filipendula ulmaria, Phyllanthus emblica, Potentilla species, okra, algae, evening primrose, pomegranate, lady's mantle, rosemary, sage, Aloe species, Echinacea, birch, apple or soya.

Substances which are particularly preferred for use as further active compounds against ageing of the skin are β-glucans, and 1,3-1,4-linked β-glucan from oats, Rubus fruticosus extract or wheat protein is particularly preferred.

The formulations according to the invention can also comprise active compounds which stimulate shading or tanning of the skin and hair in a chemical or natural manner. A faster action based on synergistic effects is thereby achieved. Substances which are particularly preferred in this context are substrates or substrate analogues of tyrosinase, such as L-tyrosine, L-DOPA or L-dihydroxyphenylalanine, stimulators of tyrosinase activity or expression, such as theophylline, caffeine, propiomelanocortin peptides, such as ACTH, alpha-MSH, peptide analogues thereof and other substances which bind to the melanocortin receptor, peptides, such as Val-Gly-Val-Ala-Pro-Gly, Lys-Ile-Gly-Arg-Lys or Leu-Ile-Gly-Lys, purines, pyrimidines, folic acid, copper salts, such as copper gluconate, chloride or pyrrolidonate, flavonoids, flavanone glycosides, such as naringin and hesperidin, melanin derivatives, such as Melasyn-100 and MelanZe, diacylglycerols, aliphatic or cyclic diols, psoralene, prostaglandins and analogues thereof, activators of adenylate cyclase and compounds which activate the transfer of melanosomes into keratinocytes, such as serine proteases or extracts from plants and plant parts of the Chrysanthemum species or Sanguisorba species, walnut extracts, urucum extracts, rhubarb extracts, erytrulose and dihydroxyacetone.

The formulations according to the invention can also be employed in combination with (further) skin-lightening active compounds. According to the invention, all the skin-lightening active compounds which are suitable or usual for cosmetic and/or dermatological uses can be used here. Advantageous skin-lightening active compounds in this respect are kojic acid (5-hydroxy-2-hydroxymethyl-4-pyranone), kojic acid derivatives, such as e.g. kojic acid dipalmitate, arbutin, ascorbic acid, ascorbic acid derivatives, hydroquinone, hydroquinone derivatives, resorcinol, sulfur-containing molecules, such as e.g. cysteine, alpha-hydroxy acids (e.g. citric acid, lactic acid, malic acid) and derivatives thereof, N-acetyl-tyrosine and derivatives, undecenoylphenylalanine, gluconic acid, 4-alkylresorcinols, 4-(1-phenylethyl)-1,3-benzenediol, chromone derivatives, such as aloesin, flavonoids, thymol derivatives, 1-aminoethylphosphinic acid, thiourea derivatives, ellagic acid, nicotinamide, zinc salts, such as e.g. zinc chloride or gluconate, thujaplicin and derivatives, triterpenes, such as maslic acid, sterols, such as ergosterol, benzofuranones, such as senkyunolide, vinyl- and ethylguaiacol, inhibitors of nitrogen oxide synthesis, such as e.g. L-nitroarginine and derivatives thereof, 2,7-dinitroindazole or thiocitrullin, metal chelators (e.g. α-hydroxy-fatty acids, palmitic acid, phytic acid, lactoferrin, humic acid, bile acid, bile extracts, bilirubin, biliverdin, EDTA, EGTA and derivatives thereof), retinoids, soya milk, serine protease inhibitors or liponic acid or other synthetic or natural active compounds for lightening of the skin and hair, the latter also being used in the form of an extract from plants, such as e.g. bearberry extract, rice extract, liquorice root extract or constituents concentrated therefrom, such as glabridin or licochalcone A, Artocarpus extract, extract from Rumex and Ramulus species, extracts from pine species (Pinus) and extracts from Vitis species or stilbene derivatives concentrated therefrom, and extract from Saxifraga, mulberry, Scutelleria or/and grape.

Advantageous skin and hair tanning active ingredients in this respect are substrates or substrate analogues of tyrosinase such as L-tyrosine, N-acetyl tyrosine, L-DOPA or L-dihydroxyphenylalanine, xanthine alkaloids such as caffeine, theobromine and theophylline and derivatives thereof, proopiomelanocortin peptides such as ACTH, alpha-MSH, peptide analogues thereof and other substances which bind to the melanocortin receptor, peptides such as Val-Gly-Val-Ala-Pro-Gly, Lys-Ile-Gly-Arg-Lys or Leu-Ile-Gly-Lys, purines, pyrimidines, folic acid, copper salts such as copper gluconate, chloride or pyrrolidonate, 1,3,4-oxadiazole-2-thiols such as 5-pyrazin-2-yl-1,3,4-oxadiazole-2-thiol, zinc diglycinate (Zn(Gly)2), manganese(II) bicarbonate complexes (“pseudocatalases”) as described for example in EP 0 584 178, tetrasubstituted cyclohexene derivatives as described for example in WO 2005/032501, isoprenoids as described in WO 2005/102252 and in WO 2006/010661, melanin derivatives such as Melasyn-100 and MelanZe, diacyl glycerols, aliphatic or cyclic diols, psoralens, prostaglandins and analogues thereof, activators of adenylate cyclase and compounds which activate the transfer of melanosomes to keratinocytes such as serine proteases, extracts of plants and plant parts of the chrysanthemum species, sanguisorba species, walnut extracts, urucum extracts, rhubarb extracts, trehalose, erythrulose and dihydroxyacetone. Flavonoids which bring about skin and hair tinting or tanning (e.g. quercetin, rhamnetin, kaempferol, fisetin, genistein, daidzein, chrysin and apigenin, epicatechin, diosmin and diosmetin, morin, quercitrin, naringenin, hesperidin, phloridzin and phloretin) can also be used.

The amount of the aforementioned examples of additional active ingredients for the modulation of skin and hair pigmentation (one or more compounds) in the products according to the invention is then preferably 0.00001 to 30 wt. %, preferably 0.0001 to 20 wt. %, particularly preferably 0.001 to 5 wt. %, based on the total weight of the preparation.

Formulations according to the invention can advantageously also comprise moisture retention regulators. The following substances e.g. are used as moisture retention regulators (“moisturizers”): sodium lactate, urea and derivatives, alcohols, glycerol, diols, such as propylene glycol, 1,2-pentanediol, 1,2-hexanediol and 1,2-octanediol, collagen, elastin or hyaluronic acid, diacyl adipates, petrolatum, urocanic acid, lecithin, panthenol, phytantriol, lycopene, (pseudo-)ceramides, glycosphingolipids, cholesterol, phytosterols, chitosan, chondroitin sulfate, lanolin, lanolin esters, amino acids, alpha-hydroxy acids (e.g. citric acid, lactic acid, malic acid) and derivatives thereof, mono-, di- and oligosaccharides, such as, for example, glucose, galactose, fructose, mannose, laevulose and lactose, polysugars, such as β-glucans, in particular 1,3-1,4-β-glucan from oats, alpha-hydroxy-fatty acids, triterpenic acids, such as betulic acid or ursolic acid, and algae extracts.

Formulations according to the invention can also be employed together with osmolytes. Osmolytes which may be mentioned by way of example are: substances from the group consisting of sugar alcohols (myo-inositol, mannitol, sorbitol), quaternary amines, such as taurine, choline, betaine, betaine-glycine and ectoin, diglycerol phosphate, phosphorylcholine, glycerophosphorylcholines, amino acids, such as glutamine, glycine, alanine, glutamate, aspartate or proline, phosphatidylcholine, phosphatidylinositol and inorganic phosphates, as well as polymers of the compounds mentioned, such as proteins, peptides, poly-amino acids and polyols. All osmolytes at the same time have a skin-moisturizing action.

Formulations according to the invention can advantageously also comprise vitamins and vitamin precursors, it being possible for all the vitamins and vitamin precursors which are suitable or usual for cosmetic and/or dermatological uses to be used. Vitamins and vitamin precursors which may be mentioned by way of example are:

vitamin A (retinol) and its derivatives (e.g. vitamin A acetate, vitamin A acid, vitamin A aldehyde, vitamin A palmitate, vitamin A propionate), vitamin B1 (thiamine) and its salts (e.g. vitamin B1 hydrochloride, vitamin B1 mononitrate, thiamine diphosphate, thiamine pyrophosphate), vitamin B12 (cobalamin), vitamin B2 (vitamin G, riboflavin) and its derivatives (e.g. vitamin B2 tetraacetate), vitamin B3 and its derivatives (e.g. nicotinamide ascorbate, nicotinamide glycollate, nicotinamide hydroxycitrate, nicotinamide lactate, nicotinamide malate, nicotinamide mandelate, nicotinamide salicylate, nicotinamide thioctate), vitamin B4 (adenine) and its derivatives (e.g. adenine riboside, disodium flavin adenine dinucleotide, nicotinamide adenine dinucleotide), provitamin B5, vitamin B5 (pantothenic acid) and its derivatives (e.g. acetyl pantothenyl ethyl ether, allantoin calcium pantothenate, allantoin DL-pantothenyl alcohol, bis(pantothenamidoethyl) disulfide, calcium pantothenate, hydroxyethyl pantothenamide MEA, sodium pantothenate, N-D-pantothenoyl-2-(2-am inoethoxy)ethanol, N-D-pantothenoyl-2-aminoethanol, N-hydroxyethoxyethyl pantothenamide, N-hydroxyethyl pantothenamide, pantothenamide MEA, pantothenol, pantothenic acid lactone, pantothenic acid polypeptide, pantothenyl ethyl ether), vitamin B6 (pyridoxol, pyroxidal, pyridoxamine) and its derivatives (e.g. pyridoxine dicaprylate, vitamin B6 dilaurate, vitamin B6 dioctanoate, vitamin B6 dipalmitate, pyridoxine glycyrrhetinate, vitamin B6 hydrochloride, vitamin B6 phosphate, vitamin B6 serine, vitamin B6 tripalmitate), vitamin C (ascorbic acid) and its derivatives (e.g. 3-O-ethyl ascorbic acid, allantoin ascorbate, aminopropyl ascorbyl phosphate, araboascorbic acid, monosodium salt, ascorbic acid palmitate, ascorbic acid polypeptide, ascorbosilane C, ascorbyl dipalmitate, ascorbyl glucoside, ascorbyl inositol nicotinate, ascorbyl linoleate, ascorbyl methylsilanol pectinate, ascorbyl nicotinamide, ascorbyl phosphate magnesium, ascorbyl stearate, ascorbyl tetraisopalmitate, ascorbyl tocopheryl maleate, calcium ascorbate, chitosan ascorbate, D-arabino-ascorbic acid, disodium ascorbyl sulfate, glucosamine ascorbate, inositol hexanicotinate hexa-ascorbate, isoascorbic acid, L-ascorbic acid, 2-(dihydrogen phosphate), trisodium salt, L-ascorbic acid, 2-[(3-cholest-5-en-3-yl hydrogen phosphate], monosodium salt, L-ascorbic acid, 2-O-D-glucopyranosyl-, L-ascorbic acid, 3-O-ethyl ether, magnesium ascorbate, magnesium ascorbylborate, methoxy PEG-7 ascorbic acid, methylsilanol ascorbate, potassium ascorbyl tocopheryl phosphate, potassium ascorbylborate, sodium ascorbate, sodium ascorbyl phosphate, sodium ascorbyl/cholesteryl phosphate, sodium isoascorbate, sodium L-ascorbyl 2-phosphate, tetrahexyldecyl ascorbate), provitamin D, vitamin D (calciol) and its derivatives (e.g. vitamin D2, vitamin D3), vitamin E (D-alpha-tocopherol) and its derivatives (e.g. di-alpha-tocopherol, polyoxypropylene/polyoxyethylene/tocopherol ether, polypropylene glycol/tocopherol ether, tocopherol cysteamine, tocopherol phosphate, sodium vitamin E phosphate, vitamin E acetate, vitamin E linoleate, vitamin E nicotinate, vitamin E succinate), vitamin F (essential fatty acids, linolenic acid and linoleic acid) and its derivatives (e.g. vitamin F ethyl ester, vitamin F glyceryl ester), vitamin H (vitamin B7, biotin), vitamin K1 (phylloquinone, phytonadione) and vitamin K3 (menadione, menaquinone).

Formulations according to the invention can likewise comprise one or more (further) plant extracts, which are conventionally prepared by extraction of the whole plant, but in individual cases also exclusively from blossom and/or leaves, wood, bark or roots of the plant. In respect of the plant extracts which can be used, reference is made in particular to the extracts which are listed in the table starting on page 44 of the 3rd edition of the Leitfaden zur Inhaltsstoffdeklaration kosmetischer Mittel [Manual of Declaration of the Constituents of Cosmetic Compositions], published by Industrieverband Körperpflegemittel and Waschmittel e.V. (IKW), Frankfurt. Extracts which are advantageous in particular are those from aloe, algae, apple, apricot, arnica, avocado, pear, stinging nettle, blackberry, calendula, ivy, hibiscus, oak bark, strawberry, spruce, honeysuckle, barley, ginkgo, ginseng, pomegranate, grapefruit, cucumber, oats, witch hazel, restharrow, henna, raspberry, elder, honeybush, hops, coltsfoot, kiwi, burdock, coconut, lavender, lime, linden, mallow, almond, mango, box holly, Melissa, olive, orange, peppermint, Pueraria, wild thyme, rooibos, rose, rosemary, horse chestnut, sage, sandalwood, yarrow, horsetail, Sophora, liquorice, dead nettle, tea (green, white, black), thyme, grape, juniper, willow, rose-bay willow-herb, hawthorn, wheat, lady's smock, cinnamon, lemon and lemongrass. In this context, the extracts from aloe vera, algae, arnica, stinging nettle, calendula, witch hazel, linden, ginseng, cucumber, rosemary and sage are particularly preferred. Mixtures of two or more plant extracts can also be employed. Extraction agents which can be used for the preparation of the plant extracts mentioned are, inter alia, water, alcohols and mixtures thereof. In this context, among the alcohols lower alcohols, such as ethanol and isopropanol, and also polyhydric alcohols, such as ethylene glycol, propylene glycol and butylene glycol, are preferred, and in particular both as the sole extraction agent and in mixtures with water. The plant extracts can be employed both in the pure and in the diluted form.

The formulations according to the invention moreover can also preferably comprise perspiration-inhibiting active compounds (antiperspirants) and odour absorbers. Perspiration-inhibiting active compounds which are employed are, above all, aluminium salts, such as aluminium chloride, aluminium hydrochloride, nitrate, sulfate, acetate etc. In addition, however, the use of compounds of zinc, magnesium and zirconium may also be advantageous. For use in cosmetic and dermatological antiperspirants, the aluminium salts and—to a somewhat lesser extent—aluminium/zirconium salt combinations have essentially proved suitable. The aluminium hydroxychlorides which are partly neutralized and therefore tolerated better by the skin, but not quite so active, are additionally worth mentioning. Alongside aluminium salts, further substances are also possible, such as, for example, a) protein-precipitating substances, such as, inter alia, formaldehyde, glutaraldehyde, natural and synthetic tannins and trichloroacetic acid, which bring about blockage of the sweat glands on the surface, b) local anaesthetics (inter alia dilute solutions of e.g. lidocaine, prilocaine or mixtures of such substances), which eliminate sympathetic supply of the sweat glands by blockade of the peripheral nerve pathways, c) zeolites of the X, A or Y type, which, alongside the reduction in secretion of perspiration, also function as adsorbents for bad odours, and d) botulinus toxin (toxin of the bacterium Chlostridium botulinum), which is also employed in cases of hyperhidrosis, a pathologically increased secretion of perspiration, and the action of which is based on an irreversible blocking of the release of the transmitter substance acetylcholine, which is relevant for secretion of perspiration.

Odour absorbers are, for example, the laminar silicates described in DE 40 09 347, and of these in particular montmorillonite, kaolinite, nontronite, saponite, hectorite, bentonite and smectite, and furthermore, for example, zinc salts of ricinoleic acid. These likewise include deodorants, bactericidal or bacteriostatic deodorizing substances, such as e.g. hexachlorophene, 2,4,4′-trichloro-2′hydroxydiphenyl ether (Irgasan), 1,6-di-(4-chlorophenylbiguanido)-hexane (chlorhexidine) and 3,4,4′-trichlorocarbanilide, as well as the active agents described in DE 37 40 186, DE 39 38 140, DE 42 04 321, DE 42 29 707, DE 42 29 737, DE 42 37 081, DE 43 09 372 and DE 43 24 219, and cationic substances, such as e.g. quaternary ammonium salts, and odour absorbers, such as e.g. ®Grillocin (combination of zinc ricinoleate and various additives) or triethyl citrate, optionally in combination with ion exchange resins.

In various cases it may also be advantageous to employ formulations according to the invention in combination with substances which are chiefly employed for inhibition of the growth of undesirable microorganisms. In this respect, alongside conventional preservatives, further active compounds which are worth mentioning, alongside the large group of conventional antibiotics, are, in particular, the products relevant for cosmetics, such as triclosan, climbazole, zinc pyrithione, ichthyol, Octopirox (1-hydroxy-4-methyl-6-(2,4,4-trimethylpentyl)-2(1H)-pyridone, 2-aminoethanol), chitosan, farnesol, octoxyglycerol, glycerol monolaurate, arylalkyl alcohols, such as e.g. phenylethyl alcohol, 3-phenyl-1-propanol, veticol or muguet alcohol, polyglycerol esters, such as e.g. polyglyceryl 3-caprylates, and aliphatic diols, such as e.g. 1,2-decanediol, or combinations of the substances mentioned, which are employed, inter alia, against underarm odour, foot odour or dandruff formation.

Formulations according to the invention can in numerous cases also advantageously comprise preservatives. Preservatives which are preferably chosen here are those such as benzoic acid and its esters and salts, 4-hydroxybenzoic acid and its esters (INCI: Parabens, preferably methylparaben, ethylparaben, butylparaben, propylparaben and/or isobutylparaben) and salts, propionic acid and its esters and salts, salicylic acid and its esters and salts, 2,4-hexadienoic acid (sorbic acid) and its esters and salts, formaldehyde and paraformaldehyde, 2-hydroxybiphenyl ether and its salts, 2-zinc-sulfidopyridine N-oxide, inorganic sulfites and bisulfites, sodium iodate, chlorobutanolum, 4-ethylmercury-(II)5-amino-1,3-bis(2-hydroxybenzoic acid), its salts and esters, dehydracetic acid, formic acid, 1,6-bis(4-amidino-2-bromophenoxy)-n-hexane and its salts, the sodium salt of ethylmercury-(II)-thiosalicylic acid, phenylmercury and its salts, 10-undecylenic acid and its salts, 5-amino-1,3-bis(2-ethylhexyl)-5-methyl-hexahydropyrimidine, 5-bromo-5-nitro-1,3-dioxane, 2-bromo-2-nitro-1,3-propanediol, 2,4-dichlorobenzyl alcohol, N-(4-chlorophenyl)-N′-(3,4-dichlorophenyl)-urea, 4-chloro-m-cresol, 2,4,4′-trichloro-2′-hydroxydiphenyl ether, 4-chloro-3,5-dimethylphenol, 1,1′-methylene-bis(3-(1-hydroxymethyl-2,4-dioximidazolidin-5-yl)urea), poly-(hexamethylened iguanide) hydrochloride, 2-phenoxyethanol, hexamethylenetetramine, 1-(3-chloroallyl)-3,5,7-triaza-1-azonia-adamantane chloride, 1-(4-chlorophenoxy)-1-(1H-imidazol-1-yl)-3,3-dimethyl-2-butanone, 1,3-bis-(hydroxy-methyl)-5,5-dimethyl-2,4-imidazolidinedione, benzyl alcohol, Octopirox, 1,2-dibromo-2,4-dicyanobutane, 2,2′-methylene-bis(6-bromo-4-chlorophenol), bromochlorophene, mixture of 5-chloro-2-methyl-3(2H)-isothiazolinone and 2-methyl-3(2H)-isothiazolinone with magnesium chloride and magnesium nitrate, 2-benzyl-4-chlorophenol, 2-chloroacetamide, chlorhexidine, chlorhexidine acetate, chlorhexidine gluconate, chlorhexidine hydrochloride, 1-phenoxy-propan-2-ol, N-alkyl(C₁₂-C₂₂)trimethylammonium bromide and chloride, 4,4-dimethyl-1,3-oxazolidine, N-hydroxymethyl-N-(1,3-di(hydroxymethyl)-2,5-dioxoimidazolidin-4-yl)-N′-hydroxy-methylurea, 1,6-bis(4-amidino-phenoxy)-n-hexane and its salts, glutaraldehyde, 5-ethyl-1-aza-3,7-dioxabicyclo(3.3.0)octane, 3-(4-chlorophenoxy)-1,2-propanediol, hyamines, alkyl-(C₈-C₁₈)-dimethyl-benzyl-ammonium chloride, alkyl-(C₈-C₁₈)-dimethyl-benzylammonium bromide, alkyl-(C₈-C₁₈)-dimethyl-benzyl-ammonium saccharinate, benzyl hemiformal, 3-iodo-2-propynyl butylcarbamate, sodium hydroxymethyl-aminoacetate or sodium hydroxymethyl-aminoacetate.

Cosmetic or dermatological formulations which comprise/are compositions according to the invention can also be in the form of emulsions.

The oily phase can advantageously be chosen from the following substance group:
mineral oils, mineral waxes
fatty oils, fats, waxes and other natural and synthetic fat substances, preferably esters of fatty acids with alcohols of low C number, e.g. with isopropanol, propylene glycol or glycerol, or esters of fatty alcohols with alkanoic acids of low C number or with fatty acids;
alkyl benzoates;
silicone oils, such as dimethylpolysiloxanes, diethylpolysiloxanes, diphenylpolysiloxanes and mixed forms thereof.

Compounds which can advantageously be employed are (a) esters of saturated and/or unsaturated branched and/or unbranched alkanecarboxylic acids having a chain length of from 3 to 30 C atoms and saturated and/or unsaturated, branched and/or unbranched alcohols having a chain length of from 3 to 30 C atoms, (b) esters of aromatic carboxylic acids and saturated and/or unsaturated, branched and/or unbranched alcohols having a chain length of from 3 to 30 C atoms. Preferred ester oils are isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 3,5,5-trimethylhexyl 3,5,5-trimethylhexanoate, 2-ethylhexyl isononanoate, 2-ethylhexyl 3,5,5-trimethylhexanoate, 2-ethylhexyl 2-ethylhexanoate, 2-ethylhexyl palmitate, 2-ethylhexyl laurate, 2-hexyldecyl stearate, 2-octyldodecyl palmitate, stearyl isononanoate, palmityl isononanoate, cetearyl isononanoate, stearyl nonanoate, palmityl nonanoate, cetearyl nonanoate, palmityl 3,5,5-trimethylhexanoate, stearyl 3,5,5-trimethylhexanoate, cetearyl 3,5,5-trimethylhexanoate, oleyl oleate, oleyl erucate, erucyl oleate, erucyl erucate and synthetic, semi-synthetic and natural mixtures of such esters, e.g. jojoba oil.

The oily phase can furthermore advantageously be chosen from the group consisting of branched and unbranched hydrocarbons and waxes, silicone oils and dialkyl ethers, the group consisting of saturated or unsaturated, branched or unbranched alcohols, and the fatty acid triglycerides, namely the triglycerol esters of saturated and/or unsaturated, branched and/or unbranched alkanecarboxylic acids having a chain length of from 8 to 24, in particular 12 to 18 C atoms. The fatty acid triglycerides can advantageously be chosen from the group consisting of synthetic, semi-synthetic and natural oils, e.g. olive oil, sunflower oil, soya oil, groundnut oil, rapeseed oil, almond oil, palm oil, coconut oil, palm kernel oil and more of the like. Any desired blends of such oil and wax components can also advantageously be employed. In some cases it is also advantageous to employ waxes, for example cetyl palmitate, as the sole lipid component of the oily phase, and the oily phase is advantageously chosen from the group which consists of 2-ethylhexyl isostearate, octyldodecanol, isotridecyl isononanoate, isoeicosane, 2-ethylhexyl cocoate, C₁₂₋₁₅-alkyl benzoate, caprylic/capric acid triglyceride and dicaprylyl ether. Mixtures of C₁₂₋₁₅-alkyl benzoate and 2-ethylhexyl isostearate, mixtures of C₁₂₋₁₅-alkyl benzoate and isotridecyl isononanoate and mixtures of C₁₂₋₁₅-alkyl benzoate, 2-ethylhexyl isostearate and isotridecyl isononanoate are particularly advantageous. The hydrocarbons paraffin oil, squalane and squalene can also advantageously be used. The oily phase can furthermore have a content of cyclic or linear silicone oils or consist entirely of such oils, it being advantageous to use an additional content of other oily phase components in addition to the silicone oil or silicone oils. Cyclomethicone (e.g. decamethylcyclopentasiloxane) can advantageously be employed as a silicone oil. However, other silicone oils, for example undecamethylcyclotrisiloxane, polydimethylsiloxane and poly(methyl-phenylsiloxane), can also advantageously be used. Mixtures of cyclomethicone and isotridecyl isononanoate and of cyclomethicone and 2-ethylhexyl isostearate are furthermore particularly advantageous.

Formulations in the form of an emulsion which comprise a formulation according to the invention advantageously comprise one or more emulsifiers. O/W emulsifiers can advantageously be chosen, for example, from the group consisting of further polyethoxylated but also polypropoxylated or further polyethoxylated and polypropoxylated products not mentioned as preferred polyethoxylated products used as component of compositions according to the present invention.

Polyethoxylated or polypropoxylated or polyethoxylated and polypropoxylated O/W emulsifiers employed are particularly advantageously chosen from the group consisting of substances having HLB values of 11-18, very particularly advantageously having HLB values of 14.5-15.5, if the O/W emulsifiers contain saturated radicals R and R′. If the O/W emulsifiers contain unsaturated radicals R and/or R′, or isoalkyl derivatives are present, the preferred HLB value of such emulsifiers can also be lower or higher. It is of advantage to choose the fatty alcohol ethoxylates from the group consisting of ethoxylated stearyl alcohols, cetyl alcohols and cetyl stearyl alcohols (cetearyl alcohols).

Advantageous W/O emulsifiers which can be employed are: fatty alcohols having 8 to 30 carbon atoms, monoglycerol esters of saturated and/or unsaturated, branched and/or unbranched alkanecarboxylic acids having a chain length of from 8 to 24, in particular 12 to 18 C atoms, diglycerol esters of saturated and/or unsaturated, branched and/or unbranched alkanecarboxylic acids having a chain length of from 8 to 24, in particular 12 to 18 C atoms, monoglycerol ethers of saturated and/or unsaturated, branched and/or unbranched alcohols having a chain length of from 8 to 24, in particular 12 to 18 C atoms, diglycerol ethers of saturated and/or unsaturated, branched and/or unbranched alcohols having a chain length of from 8 to 24, in particular 12 to 18 C atoms, propylene glycol esters of saturated and/or unsaturated, branched and/or unbranched alkanecarboxylic acids having a chain length of from 8 to 24, in particular 12 to 18 C atoms and sorbitan esters of saturated and/or unsaturated, branched and/or unbranched alkanecarboxylic acids having a chain length of from 8 to 24, in particular 12 to 18 C atoms.

Formulations according to the invention for cosmetic (topical) prophylactic (preventive) treatment of the skin can regularly comprise a high content of care substances. According to a preferred embodiment, the compositions comprise one or more animal and/or plant fats and oils having care properties, such as olive oil, sunflower oil, refined soya oil, palm oil, sesame oil, rapeseed oil, almond oil, borage oil, evening primrose oil, coconut oil, shea butter, jojoba oil, oat oil, sperm oil, beef tallow, neat's foot oil and lard, and optionally further care constituents, such as, for example, fatty alcohols having 8-30 C atoms. The fatty alcohols here can be saturated or unsaturated and linear or branched. Alcohols which can be employed are, for example, decanol, decenol, octanol, octenol, dodecanol, dodecenol, octadienol, decadienol, dodecadienol, oleyl alcohol, ricinoleyl alcohol, erucyl alcohol, stearyl alcohol, isostearyl alcohol, cetyl alcohol, lauryl alcohol, myristyl alcohol, arachidyl alcohol, caprylyl alcohol, capryl alcohol, linoleyl alcohol, linolenyl alcohol and behenyl alcohol, and Guerbet alcohols thereof, it being possible for the list to be extended virtually as desired by further alcohols of related structural chemistry. The fatty alcohols preferably originate from natural fatty acids, being conventionally prepared from the corresponding esters of the fatty acids by reduction.

Fatty alcohol fractions which are formed by reduction from naturally occurring fats and fatty oils, such as e.g. beef tallow, groundnut oil, colza oil, cottonseed oil, soya oil, sunflower oil, palm kernel oil, linseed oil, maize oil, castor oil, rape oil, sesame oil, cacao butter and coconut fat, can furthermore be employed.

Care substances which can be combined in an outstanding manner with formulations according to the invention moreover also include
waxes, such as e.g. candelilla wax or carnauba wax
ceramides, where ceramides are understood as meaning N-acylsphingosins (fatty acid amides of sphingosin) or synthetic analogues of such lipids (so-called pseudoceramides), which significantly improve the water retention capacity of the stratum corneum.
phospholipids, for example soya lecithin, egg lecithin and cephalins
vaseline, paraffin oils and silicone oils; the latter include, inter alia, dialkyl- and alkylarylsiloxanes, such as dimethylpolysiloxane and methylphenylpolysiloxane, as well as alkoxylated and quaternized derivatives thereof.

According to a preferred aspect, the present invention also relates to a composition according to the invention (as described above)
a) for use in the treatment or prevention of one or more itchy skin conditions, and/or
b) for use as PAR-2 antagonist.

In accordance, the invention also relates to the use, preferably non-therapeutic use, of a composition according to the invention (as described above)
a) for preventing, reducing or alleviating one or more itchy skin conditions, and/or
b) as PAR-2 antagonist.

In some embodiments, in particular for medical purposes, it is advantageous to administer a composition according to the present invention orally e.g. in the form of (compressed) tablets, dragees, comprimates, powders, capsules, juices, solutions and granules or in form of orally consumable products used for alimentation which in addition to their function as foodstuff provide beauty from inside.

The invention also provides the use of one or more compounds of formula 1 and/or respective salt(s) thereof or a composition according to the invention for reducing, eliminating or suppressing the skin-itch effect of a substance or substance mixture.

As described above, compounds of the general formula 1 can be formulated where applicable as pharmaceutically acceptable salt(s). Pharmaceutically acceptable salts particularly include those salts prepared by reaction of compounds of the general formula 1 where applicable with a pharmaceutically acceptable inorganic base. Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like. The potassium and sodium salt forms are particularly preferred. It should be recognized that the particular counterion forming a part of any salt of this invention is usually not of a critical nature, so long as the salt as a whole is pharmacologically acceptable and as long as the counterion does not contribute undesired qualities to the salt as a whole.

The pharmaceutical compositions are, preferably, administered topically or systemically. Suitable routes of administration conventionally used for drug administration are oral, intravenous, dermal or parenteral administration as well as inhalation.

The pharmaceutical carrier must be acceptable in the sense of being compatible with the other ingredients of the formulation and being not deleterious to the recipient thereof. The pharmaceutical carrier employed shall be, preferably, a solid, a gel or a liquid. Preferred pharmaceutical solid carriers are lactose, terra alba, sucrose, talc, gelatin, agar, pectin, acacia, magnesium stearate, stearic acid and the like. Preferred liquid carriers are phosphate buffered saline solution, syrup, oil such as peanut oil and olive oil, water, emulsions, various types of wetting agents, sterile solutions and the like. Similarly, the carrier may include time delay material well known to the art, such as glyceryl monostearate or glyceryl di-stearate alone or with a wax. Further suitable carriers are well known in the art, see, e.g., Remington's Pharmaceutical Sciences, Mack Publishing Company, Easton, Pa. For particularly preferred carriers, see above.

The diluent is, preferably, selected so as not to affect the biological activity of the combination. Preferred diluents are distilled water, physiological saline, Ringer's solutions, dextrose solution, and Hank's solution.

The pharmaceutical composition or formulation, preferably, may comprise more than one of the aforementioned carriers or diluents as well as other components such as adjuvants or non-toxic, non-therapeutic, non-immunogenic stabilizers and the like.

A therapeutically effective dosage or amount refers to an amount of compounds of the general formula 1 in a pharmaceutical composition of the present invention which prevents, ameliorates or treats the symptoms accompanying a disease or condition referred to in this specification.

Moreover, a therapeutically effective dosage can be also described by the IC50 value, i.e. the amount of a therapeutically active compound which is required to achieve half of the maximum inhibition for an enzyme or signalling molecule, such as PAR-2 in the present case.

The dosage regimen will be determined by the attending physician and other clinical factors; preferably in accordance with any one of the above described methods. As is well known in the medical arts, dosages for any one patient depends upon many factors, including the patient's size, body surface area, age, sex, time and route of administration, general health, and other drugs being administered concurrently. Progress can be monitored by periodic assessment.

The pharmaceutical compositions referred to herein are administered at least once in order to treat or ameliorate or prevent a disease or condition recited in this specification. However, the said pharmaceutical compositions may be administered more than one time, for example from one to four times daily up to a non-limited number of days.

A pharmaceutical composition of the present invention can for example be formulated as a capsule, sachet, cachet, paper or other suitable container or vehicle. The resulting formulations are to be adapted to the mode of administration, i.e. in the forms of tablets, capsules, suppositories, solutions, suspensions or the like. Dosage recommendations shall be indicated in the prescribers or users instructions in order to anticipate dose adjustments depending on the considered recipient. The aforementioned carriers or diluents may for example be present in amounts of 1 to 99% weight (w/w) or even more, preferably of 10 to 80% weight (w/w) based on the total weight of the envisaged composition. The required amounts of the substances or additives can be determined by those skilled in the art without further ado, e.g. by trial and error, dependent on the envisaged formulation and its application provided that the formulation provides a therapeutically effective dosage of compounds of the general formula 1 as discussed above.

Moreover, compounds of the general formula 1 can be administered in combination with other substances, such as drugs or cosmetic agents, either in a common pharmaceutical composition or as separated pharmaceutical compositions wherein said separated pharmaceutical compositions may be provided in form of a kit of parts.

A further aspect of the present invention relates to formulations (compositions) according to the invention in the form of oral care products (oral hygiene products), wherein the oral care product is preferably in the form of toothpaste, dental cream, dental gel, dental powder, tooth-cleaning liquid, tooth-cleaning foam, mouthwash, dental cream and mouthwash as a 2-in-1 product, sweet for sucking, mouth spray, dental silk or dental care chewing gum. The activity of the formulations according to the invention also manifests itself remarkably well in the field of oral hygiene. A bad breath-reducing activity of the formulations according to the invention has moreover been found in our own studies.

Dental care compositions (as a preferred example of an oral care product according to the invention) in general comprise an abrasive system (abrasive or polishing agent), such as e.g. silicas, calcium carbonates, calcium phosphates, aluminium oxides and/or hydroxyapatites, surface-active substances, such as e.g. sodium lauryl sulfate, sodium lauryl sarcosinate and/or cocamidopropyl betaine, moisture-retaining agents, such as e.g. glycerol and/or sorbitol, thickening agents, such as e.g. carboxymethylcellulose, polyethylene glycols, carrageenan and/or Laponite®, sweeteners, such as e.g. saccharin, flavour correctants for unpleasant taste impressions, flavour correctants for further, as a rule not unpleasant taste impressions, flavour-modulating substances (e.g. inositol phosphate, nucleotides, such as guanosine monophosphate, adenosine monophosphate or other substances, such as sodium glutamate or 2-phenoxypropionic acid), cooling agents, such as e.g. menthol derivatives (e.g. L-menthyl lactate, menthyl ethylamido oxalate, L-menthyl alkyl carbonates, menthone ketals, menthanecarboxylic acid amides), 2,2,2-trialkylacetic acid amides (e.g. 2,2-diisopropylpropionic acid methylamide), icilin and icilin derivatives, stabilizers and active compounds, such as e.g. sodium fluoride, sodium monofluorophosphate, tin difluoride, quaternary ammonium fluorides, zinc citrate, zinc sulfate, tin pyrophosphate, tin dichloride, mixtures of various pyrophosphates, triclosan, cetylpyridinium chloride, aluminium lactate, potassium citrate, potassium nitrate, potassium chloride, strontium chloride, hydrogen peroxide, aromas, sodium bicarbonate and/or odour correctants.

Formulations according to the invention in the form of chewing gums or dental care chewing gums comprise chewing gum bases which comprise elastomers, such as, for example, polyvinyl acetates (PVA), polyethylenes, (low or medium molecular weight) polyisobutenes (PIB), polybutadienes, isobutene-isoprene copolymers (butyl rubber), polyvinyl ethyl ethers (PVE), polyvinyl butyl ethers, copolymers of vinyl esters and vinyl ethers, styrene/butadiene copolymers (styrene/butadiene rubber, SBR) or vinyl elastomers, e.g. based on vinyl acetate/vinyl laurate, vinyl acetate/vinyl stearate or ethylene/vinyl acetate, and mixtures of the elastomers mentioned, as described, for example, in EP 0 242 325, U.S. Pat. No. 4,518,615, U.S. Pat. No. 5,093,136, U.S. Pat. No. 5,266,336 U.S. Pat. No. 5,601,858 or U.S. Pat. No. 6,986,709. In addition, chewing gum bases comprise further constituents, such as, for example, (mineral) fillers, plasticizers, emulsifiers, antioxidants, waxes, fats or fatty oils, such as, for example, hardened (hydrogenated) plant or animal fats, and mono-, di- or triglycerides. Suitable (mineral) fillers are, for example, calcium carbonate, titanium dioxide, silicon dioxide, talc, aluminium oxide, dicalcium phosphate, tricalcium phosphate, magnesium hydroxide and mixtures thereof. Suitable plasticizers or agents for preventing sticking (detackifiers) are, for example, lanolin, stearic acid, sodium stearate, ethyl acetate, diacetin (glycerol diacetate), triacetin (glycerol triacetate) and triethyl citrate. Suitable waxes are, for example, paraffin waxes, candelilla wax, carnauba wax, microcrystalline waxes and polyethylene waxes. Suitable emulsifiers are, for example, phosphatides, such as lecithin, and mono- and diglycerides of fatty acids, e.g. glycerol monostearate.

Formulations according to the invention (in particular those which are in the form of an oral care product) preferably additionally comprise one or more aroma and/or flavouring substances, such as essential oils and extracts, tinctures and balsams, such as, for example, anisole, basil oil, bergamot oil, bitter almond oil, camphor oil, citronella oil, lemon oil; Eucalyptus citriodora oil, eucalyptus oil, fennel oil, grapefruit oil, ginger oil, camomile oil, spearmint oil, caraway oil, lime oil, mandarin oil, nutmeg oil (in particular nutmeg blossom oil=maces oil, mace oil), myrrh oil, clove oil, clove blossom oil, orange oil, oregano oil, parsley (seed) oil, peppermint oil, rosemary oil, sage oil (clary sage, Dalmatian or Spanish sage oil), star aniseed oil, thyme oil, vanilla extract, juniper oil (in particular juniper berry oil), wintergreen oil, cinnamon leaf oil; cinnamon bark oil, and fractions thereof, or constituents isolated therefrom.

It is of particular advantage if the formulations according to the invention comprise at least one aroma substance, preferably 2, 3, 4, 5, 6, 7, 8, 9, 10 or more aroma substances, chosen from the following group: menthol (preferably I-menthol and/or racemic menthol), anethole, anisole, anisaldehyde, anisyl alcohol, (racemic) neomenthol, eucalyptol (1,8-cineol), menthone (preferably L-menthone), isomenthone (preferably D-isomenthone), isopulegol, menthyl acetate (preferably L-menthyl acetate), menthyl propionate, carvone (preferably (−)-carvone, optionally as a constituent of a spearmint oil), methyl salicylate (optionally as a constituent of a wintergreen oil), eugenol acetate, isoeugenol methyl ether, beta-homocyclocitral, eugenol, isobutyraldehyde, 3-octanol, dimethyl sulfide, hexanol, hexanal, trans-2-hexenal, cis-3-hexenol, 4-terpineol, piperitone, linalool, 8-ocimenyl acetate, isoamyl alcohol, isovaleraldehyde, alpha-pinene, beta-pinene, limonene (preferably D-limonene, optionally as a constituent of an essential oil), piperitone, trans-sabinene hydrate, menthofuran, caryophyllene, germacrene D, cinnamaldehyde, mint lactone, thymol, gamma-octalactone, gamma-nonalactone, gamma-decalactone, (1,3E,5Z)-undecatriene, 2-butanone, ethyl formate, 3-octyl acetate, isoamyl isovalerate, cis- and trans-carvyl acetate, p-cymol, damascenone, damascone, cis-rose oxide, trans-rose oxide, fenchol, acetaldehyde diethyl acetal, 1-ethoxyethyl acetate, cis-4-heptenal, cis-jasmone, methyl dihydrojasmonate, 2′-hydroxypropiophenone, menthyl methyl ether, myrtenyl acetate, 2-phenylethyl alcohol, 2-phenylethyl isobutyrate, 2-phenylethyl isovalerate, geraniol, nerol and viridiflorol.

Particularly preferred cooling agents for oral care compositions according to the present invention comprise one or more cooling agents selected from the group consisting of: menthone glycerol acetal (trade name: Frescola® MGA), menthyl lactate (preferably l-menthyl lactate, menthyl ethylamido oxalate, in particular l-menthyl l-lactate, trade name: Frescolat® ML), substituted menthyl-3-carboxylic acid amides (e.g. menthyl-3-carboxylic acid N-ethylamide), 2-isopropyl-N-2,3-trimethylbutanamide, 3-menthoxypropane-1,2-diol, 2-hydroxyethyl menthyl carbonate, 2-hydroxypropyl menthyl carbonate, isopulegol, monomenthyl succinate and monomenthyl glutarate.

Formulations according to the invention which comprise l-menthol and at least one, particularly preferably at least two cooling substances are preferred according to the invention.

The invention furthermore provides a method for prophylaxis of the itchy skin action or for reducing, eliminating or suppressing the itchy skin action of a substance or substance mixture, with the following steps:
a) provision of a substance or substance mixture having a itchy skin action,
b) provision of one or more compounds of the general formula 1 or, if aplllicable, a cosmetically or pharmaceutically acceptable salt thereof, in particular the Na⁺, K⁺, NH₄⁺, Mg²⁺ or Ca²⁺ salt, or of a composition according to the invention, and
c) bringing together the substances of a) and b), so that the itchy skin and scalp action is reduced, eliminated or suppressed and a or, respectively, another composition according to the invention is formed.

One advantage of the method according to the invention is that the itchy skin action of substances or substance mixtures can be moderated in this way to the extent that they are accessible for uses for which they were hitherto not available. On the basis of the method according to the invention mentioned last, higher concentrations of itch causing substances and substance mixtures can also be employed in uses where there is the possibility of skin contact. In this context, it is particularly preferable if, on the basis of the method according to the invention mentioned last, the itchy skin and scalp action of the skin-itch causing compound is eliminated completely (i.e. it no longer exists) or is suppressed completely (i.e. it no longer has an effect). The can be employed, for example, against the itchy skin and scalp action of e.g. histamines 1-4, NK-1 agonists like substance P, mast cell destabilisers, tryptase and trypsin and further PAR-2 activating enzymes as well as SLIGR and SLIGRL or further PAR-2 activating oligopeptides

Preferred embodiments and further aspects of the present invention emerge from the attached claims and the following examples, the examples not being intended to limit the invention. Unless indicated otherwise, all data, in particular percentages, refer to the weight.

### EXAMPLES

### Example 1

### Preparation of Samples of Diarylheptanoids of the General Formula 1

Diarylheptanoids of the general formula 1 were either purchased form commercial sources, or they were synthesized.

### Example 1.1

### Synthesis of 1,7-Bis-(4-methoxyphenyl)-3,5-heptanedione (formula 3)

### a) Synthesis of 1,7-Bis-(4-methoxyphenyl)-hepta-1,6-dien-3,5-dione (formula 7)

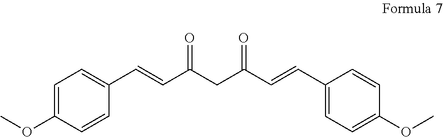

100 g (1.0 Mol) 2,4-pentandione are dissolved in 100 g ethyl acetate and then 48 g (0.7 Mol) of boric anhydride are added under stirring (slightly exothermic). After 15 min the mixture is heated to 78° C. for completion of generating the boron complex. After cooling down to 40° C. the addition of 400 g ethyl acetate, 272 g (2.0 Mol) anisaldehyde and 208 g (2.0 Mol) tributyl borate follows under continuous stirring. At last 44 g (0.6 Mol) n-butylamine are added during 1 h and the reaction mixture turns into a dark red colour. After 5 h stirring at r.t. the red coloured precipitate is filtered and washed twice with 500 g ethyl acetate and then is transferred into 0.75% aqueous sulfuric acid and stirred for 1 h at 60° C. The orange coloured precipitate is filtered, washed twice with water and then 270 g wet product are achieved.

### b) Synthesis of 1,7-Bis-(4-methoxyphenyl)-3,5-heptanedione (formula 3)

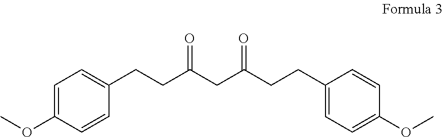

270 g of the wet cake obtained in experiment 1.1.a is dissolved in 1600 g isopropanol, 5 g Raney Nickel are added and then this mixture is hydrogenated at 5 bar and 50-55° C. during 6 h. After filtration of the catalyst the product is crystallized from the solution by cooling down to 20° C. The white product is filtered, dried and 175 g product are achieved (purity >95%, yield 50% over two steps).

### Example 1.2

### Synthesis of 1,7-Bis-(4-hydroxy-3-methoxyphenyl)-hepta-1,6-dien-3,5-dione (formula 8)

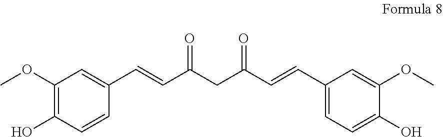

This derivative is synthesized in the same procedure as described under 1.1.a using vanillin instead of anisaldehyde as starting material

### Example 1.3

### Synthesis of 1,7-Bis-(4-hydroxy-3-methoxyphenyl)-3,5-heptanedione (formula 4))

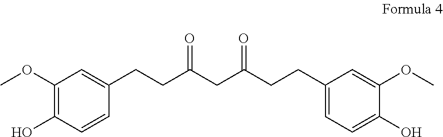

This compound is synthesized in the same procedure as described in 1.1.b) (purity >95%, yield 45% over two steps).

### Example 2

### In Vitro PAR-2 Assay

Cell-based calcium assay for the identification of PAR-2 antagonists based on fluorescent dye Fluo4-AM

Functional modulation of PAR-2 by the tested compounds was measured and quantified using the calcium sensitive fluorescent probe Fluo-4 AM on a fluorescence microplate reader. Activation of PAR-2 by an agonist (SLIGRL) led to an increase in the intracellular calcium concentration and thus an increase in fluorescence intensity. Inhibition of the PAR-2 related bioactivity by an antagonist reduced an agonist-evoked increase in fluorescence intensity significantly or preferably blocked the agonist-evoked signal completely.

### Procedure:

Human embryonic kidney 293 cells (HEK293) endogenously expressing PAR-2 were cultured in DMEM (high glucose) supplemented with tetracycline-free FCS (10% v/v), L-glutamine (4 mM), blasticidin (15 μg/ml), zeocin (100 μg/ml) and puromycin (2 μg/ml) in a water-saturated atmosphere at 37° C. and 5% CO₂.

Cells were seeded onto 96-well clear-bottom black-walled assay plates at a density of 45,000 cells per well in 100 μl of cell culture medium. Intracellular calcium increase in the living cells due to receptor activation was monitored 24 h later using the calcium sensitive fluorescent probe Fluo-4 AM on a fluorescence microplate reader (FlexStation® system, Molecular Devices). Therefore 100 μl Krebs-HEPES (KH) buffer (118 mM NaCl; 4.7 mM KCl; 1.3 mM CaCl₂; 1.2 mM MgSO₄; 1.2 mM KH₂PO₄; 4.2 mM NaHCO₃; 10 mM Hepes, pH 7.4) supplemented with sulfinpyrazone (250 μM) and Fluo-4 AM (4 μM) were added and the cells were incubated for an additional hour in a water-saturated atmosphere at 37° C. and 5% CO₂.

### Measurement of Antagonistic Activity:

Medium was replaced with 150 μl KH buffer supplemented with sulfinpyrazone (250 μM). Subsequently, 50 μl KH buffer supplemented with the screening compounds (final concentration of 10 μM under measurement conditions) or control substances were added and the cells were incubated for 10 min. under assay conditions in the microplate reader. Changes in fluorescence were recorded at 37° C. after addition of 50 μl KH buffer supplemented with the PAR-2 peptide agonist SLIGRL (final concentration of 2 μM under measurement conditions).

### Analysis:

Calcium mobilization was quantified as the change of peak fluorescence (ΔF) over the baseline level (F). The data was analyzed with the software of the microplate reader. Potential PAR-2 antagonists were tested in an effective range of 1-100 μM for their capacity to reduce the SLIGRL-evoked signal.

### Assay Principle:

A cellular in vitro assay was developed with human embryonic kidney 293 cells (HEK293) endogenously expressing PAR-2. The PAR-2 activating peptide SLIGRL (EC50: ˜0.6 μM) was used as an agonistic stimulus with subsequent Fluo-4 dependent intracellular calcium detection (calcium imaging).

Compounds of the general formula 1, significantly reducing the SLIGRL mediated PAR-2 activation, were evaluated for dose response effects and for determination of IC50 values of PAR-2 antagonistic activity.

Validation analysis on PAR-2 specificity of identified antagonists was conducted with HEK293-sh20 cells stably expressing a non-specific random shRNA (sh20) versus HEK293-sh65 cells stably overexpressing a specific, PAR-2 targeting shRNA (sh65). Compounds of the general formula 1 proved to be highly specific PAR-2 antagonists as a significant shift in IC50 values towards lower concentrations regarding PAR-2 inhibition in HEK293-sh65 cells compared to the HEK293-sh20 cells was observed.

This clearly indicated that compounds of the general formula 1 exhibit their skin soothing and, respectively, anti-itch activity via highly specific inhibition of downstream PAR-2 signaling pathways.

### Example 2.1

### PAR-2 antagonistic activity of 1,7-Bis(4-methoxyphenyl)-3,5-heptanedione (formula 3) in cell based fluorescent analysis

In the presence of 2 μM SLIGRL the sample tested for PAR-2 antagonism delivered a dose dependent inhibition with an IC₅₀ of 67.8 μM.

IC₅₀ values in the range of 25 μM to 100 μM were measured for further diarylheptanoids of the general formulae 1 and 2, like 1,7-Bis(4-methoxyphenyl)-hepta-1,6-dien-3,5-dione (formula 7), 1,7-Bis-(4-hydroxy-3-methoxyphenyl)-3,5-heptanedione (formula 8) and 1,7-Bis-(4-hydroxy-3-methoxyphenyl)-3,5-heptanedione (formula 4) as well as for plant extracts or plant extracts treated by catalytic hydrogenation for the production of extracts comprising a high amount of partially hydrogenated diarylheptanoids of general formula 2.

### Example 2.2

Amplified PAR-2 antagonistic activity in cell based fluorescent analysis of a composition according to this invention, comprising 2% 1,7-Bis(4-methoxyphenyl)-3,5-heptanedione (formula 3) and 98% of a 2:1 mixture (w/w) of PEG-9 Tridecylether and PEG5-Isonanoate (covered by general formulae 5 and 6 of the present invention).

In the presence of 2 μM SLIGRL the combination tested for PAR-2 antagonism delivered a dose dependent inhibition with an IC₅₀ of 9.4 μM.

Examples 2.1. and 2.2 delivered the unequivocal proof that the PAR-2 antagonistic activity of a combination comprising i) 2% 1,7-Bis(4-methoxyphenyl)-3,5-heptanedione (formula 3) and ii) 98% of a 2:1 mixture (w/w) of PEG-9 Tridecylether and PEG5-Isonanoate (covered by general formulae 5 and 6) is significantly increased. According to example 2.1., 1,7-Bis(4-methoxyphenyl)-3,5-heptanedione (formula 3) inhibited SLIGRL induced PAR-2 activation with an IC₅₀ of only 67.8 μM. According to example 2.2., the combination comprising i) 2% 1,7-Bis(4-methoxyphenyl)-3,5-heptanedione (formula 3) and ii) 98% of a 2:1 mixture (w/w) of PEG-9 Tridecylether and PEG5-Isonanoate inhibited SLIGRL induced PAR-2 activation with a highly significant, much lower IC₅₀ of 9.4 μM. The IC₅₀ value recorded in example 2.2. corresponds to a 7.25-fold PAR-2 inhibition compared to example 2.1.

IC₅₀ values in the range of 5 μM to 25 μM were measured for further combinations comprising diarylheptanoids like 1,7-Bis(4-methoxyphenyl)-hepta-1,6-dien-3,5-dione (formula 7), 1,7-Bis-(4-hydroxy-3-methoxyphenyl)-3,5-heptanedione (formula 8) and 1,7-Bis-(4-hydroxy-3-methoxyphenyl)-3,5-heptanedione (formula 4) and polyethylene glycol esters of the formula 5 or polyethylene glycol ethers of the formula 6 or combinations of polyethylene glycol esters of the formula 5 and polyethylene glycol ethers of the formula 6 according to the present invention.

The highly significant potency increase of combinations comprising compounds of the general formulae 1 and, respectively, 2 and polyethylene glycol esters of the general formula 5 or polyethylene glycol ethers of the general formula 6 or combinations of polyethylene glycol esters of the general formula 5 and polyethylene glycol ethers of the general formula 6 can be explained i) by a significant improvement of the solubility of hardly soluble compounds of the general formulae 1 and 2 and/or ii) by significant improvement of the bioavailability of the compounds of the general formulae 1 and 2. However, an (additional) modulation of the PAR-2 antagonistic effect of polyethylene glycol esters of the general formula 5 or polyethylene glycol ethers of the general formula 6 or combinations of polyethylene glycol esters of the general formula 5 and polyethylene glycol ethers of the general formula 6 directly at the PAR-2 receptor via e.g. allosteric effects cannot be completely excluded.

### Example 3

### Clinical Study

The aim of the double blinded clinical cross-over study was to evaluate and to compare the efficacy and the skin compatibility and tolerability of two exemplary anti-itch lotions (lotion GS11105SL-A comprising 0.2% 1,7-Bis(4-methoxyphenyl)-3,5-heptanedione, formula 3, versus placebo lotion GS11105SL-B, ethanol-propylene glycol-water solutions: ratio 5:3:2 w/w/w) for the scalp, i.e. skin of the scalp, both after a single application and after repeated applications.

It was carried out in a temperature and humidity-controlled room (24+/−2° C.; 50+/−10% R.U.). 20 volunteers suffering from itching on the scalp were selected for the study. At the beginning of the test they were divided into two groups of 10 people each: the first group began the test with GS11105SL-A lotion comprising 0.2% 1,7-Bis(4-methoxyphenyl)-3,5-heptanedione (formula 3), while the second group tested first GS11105SL-B lotion. The evaluations of dandruff, seborrhoea, erythema, burning and itching were performed on the basis of the following 4-point clinical scale: 0=absent, 1=mild, 2=moderate, 3=severe. After the basal evaluations the testing engineer applied 2 ml of GS11105SL-A lotion comprising 0.2% 1,7-Bis(4-methoxyphenyl)-3,5-heptanedione (formula 3) or GS11105SL-B lotion to each volunteer's scalp. The volunteers were then asked to score the decrease in itching sensation in the short time, after 5, 15 and 60 minutes, according to the scale: 0=no decrease, 1=poor decrease, 2=moderate decrease, 3=good decrease. Then, each volunteer was given the assigned lotion to be applied once a day for 5 days. After 5 days the subjects came back to the lab for the control visit. Thereafter they underwent a 9 days' wash-out period during which they were asked not to use any anti-itching treatments on the scalp. After the wash-out period the subjects came back to the lab to receive the second lotion according to the same procedure described above: basal visit, short term evaluation of the itching decrease, 5 days' use of the product and final clinical assessment. The dermatologist gave also a judgement about the overall tolerability of the treatment, according to a 3-point scale: 1=poor tolerability; 2=moderate tolerability; 3=good tolerability.

The comparison between the two products shows that the immediate itching decrease is more intense for product GS11105SL-A comprising 0.2% 1,7-Bis(4-methoxyphenyl)-3,5-heptanedione (formula 3) than for GS11105SL-B (statistically significant difference at T_{5 mins}; p<0.05). With the passing of time the decrease is always higher for GS11105SL-A comprising 0.2% 1,7-Bis(4-methoxyphenyl)-3,5-heptanedione.

TABLE 1
Double blinded clinical evaluation of an instant anti-itch effect
of lotions GS11105SL-A (comprising 0.2% 1,7-Bis(4-methoxyphenyl)-
3,5-heptanedione, formula 3) and GS11105SL-B (placebo); data received
5, 15 and 60 minutes after a single application

| | GS11105SL-A | | |
|---|---|---|---|
| | (0.2% 1,7-Bis(4- | | Wilcoxon |
| | methoxyphenyl)-3,5- | GS11105SL-B | test |
| Itching decrease | heptanedione, formula 3) | (Placebo) | A vs B |
| after 5 minutes | mean 1.4 | mean 0.6 | p < 0.05 |
| | std. dev. 1.3 | std. dev. 0.8 | |
| after 15 minutes | mean 1.4 | mean 0.8 | p > 0.05 |
| | std. dev. 1.2 | std. dev. 0.9 | |
| after 60 minutes | mean 1.2 | mean 0.9 | p > 0.05 |
| | std. dev. 1.2 | std. dev. 0.8 | |

Also after the 5 days repeated application period, a statistically significant itching decrease was observed for product GS11105SL-A comprising 0.2% of PAR-2 antagonist 1,7-Bis(4-methoxyphenyl)-3,5-heptanedione (formula 3, Tₒ vs T_{5 days}; p<0.01; tab.2).

No statistically significant variation after 5 days was found for dandruff, seborrhoea, erythema and burning sensation. This proved that the use of the product neither had negative effects nor induced any irritation on skin. On the basis of the clinical assessment the product was well tolerated. Dermatologist assessment resulted in a tolerability of mean 2.8 (std. dev. 0.4) for the product on the whole according to the scale 1=poor tolerability, 2=moderate tolerability, 3=good tolerability. The placebo formulation used in this double blinded study (Product GS11105SL-B; data not shown) was also well tolerated, however showed a less significant itching decrease from mean 1.5 to just mean 1.0 (Tₒ vs T_{5 days}; p<0.05).

TABLE 2
Double blinded clinical evaluation of anti-itch lotion GS11105SL-A
comprising 0.2% 1,7-Bis(4-methoxyphenyl)-3,5-heptanedione (formula
3); data received after 5 days repeated application

| GS11105SL-A | | | |
|---|---|---|---|
| (0.2% 1,7-Bis(4- | | | |
| methoxyphenyl)-3,5- | | | Wilcoxon test/ |
| heptanedione) | T₀ | T_{5 days} | t-test |
| Dandruff | mean 0.8 | mean 0.8 | T_{0 vs} T_{5 days} |
| | std. dev. 1.0 | std. dev. 0.9 | p > 0.05 |
| Seborrhoea | mean 1.1 | mean 1.3 | T_{0 vs} T_{5 days} |
| | std. dev. 0.9 | std. dev. 1.0 | p > 0.05 |
| Erythema | mean 0.7 | mean 0.6 | T_{0 vs} T_{5 days} |
| | std. dev. 0.8 | std. dev. 0.8 | p > 0.05 |
| Itching | mean 1.8 | mean 1.0 | T_{0 vs} T_{5 days} |
| | std. dev. 0.6 | std. dev. 0.9 | p < 0.01 |
| Burning | mean 0.2 | mean 0.1 | T_{0 vs} T_{5 days} |
| | std. dev. 0.4 | std. dev. 0.2 | p > 0.05 |

In summary, the in vivo efficacy of 1,7-Bis(4-methoxyphenyl)-3,5-heptanedione (formula 3) is shown by a significant decrease in itching, instantly after a single application and after 5 days repeated application, respectively. The good compatibility and tolerability on the skin is exemplary shown by non significant variations in such parameters as dandruff, erythema, seborrhoea and burning sensation after repeated application.

An even higher decrease in itch sensation, instantly after a single application and after repeated application was also determined in the clinical studies for compositions according to the invention, comprising (i) compounds of formula 1, preferably 1,7-Bis-(4-methoxyphenyl)-3,5-heptanedione (formula 3), 1,7-Bis(4-methoxyphenyl)-hepta-1,6-dien-3,5-dione (formula 7), 1,7-Bis-(4-hydroxy-3-methoxyphenyl)-3,5-heptanedione (formula 8) and/or 1,7-Bis-(4-hydroxy-3-methoxyphenyl)-3,5-heptanedione (formula 4) and (ii) one or more polyethylene glycol esters of the formula 5 or one or more polyethylene glycol ethers of the formula 6 or combinations of one or more polyethylene glycol esters of the formula 5 and one or more polyethylene glycol ethers of the formula 6.

### Formulation Examples 1-16

### Formulations (Compositions) Comprising Compounds According to Formula 1 Having (Skin Soothing) Itch-Reducing Action

### Formulation Example 1

### Skin Lightening Day Cream o/w

### Formulation Example 2

### Skin-Soothing Lotion

### Formulation Example 3

### After Sun Balm, Itch Reducing

### Formulation Example 4

### Calming Body Spray

### Formulation Example 5

### Sunscreen Lotion (o/w, Broadband Protection)

### Formulation Example 6

### W/o Night Cream

### Formulation Example 7

### Scalp Soothing Anti Dandruff Shampoo

### Formulation Example 8

### Self Tanning Cream

### Formulation Example 9

### Anti Itch Barrier Repair Cream

### Formulation Example 10

### Antiperspirant/Deodorant Roll-on

### Formulation Example 11

### Emulsion with UV-A/B-Broadband Protection

### Formulation Example 12

### Sun Spray with UV-A/B-Broadband Protection with Low Oil Content

### Formulation Example 13

### Skin-Lightening Balm with UV-A/UV-B Protection

### Formulation Example 14

### Calming Shampoo with Skin-Lightening Properties

### Formulation Example 15

### Scalp Soothing Hair Conditioner with UV-B/UV-A Protection, Rinse Off

### Formulation Example 16

### Anti Itch Hair Conditioner, Leave on

| | | % BY WEIGHT/FORMULATION EXAMPLE | | | | | | | |
|---|---|---|---|---|---|---|---|---|---|
| RAW MATERIAL NAME | INCI | 1 | 2 | 3 | 4 | 5 | 6 | 7 | 8 |
| 1,7-Bis(4-methoxyphenyl)- | 1,7-Bis(4-methoxyphenyl)- | 0.1 | 0.1 | 0.5 | 0.2 | | 0.04 | 0.1 | 0.1 |
| 3,5-heptanedione | 3,5-heptanedione | | | | | | | | |
| 1,7-Bis-(4-hydroxy-3- | Tetrahydrocurcumin | | | | | 0.06 | | 0.1 | |
| methoxyphenyl)- | | | | | | | | | |
| 3,5-heptanedione″ | | | | | | | | | |
| Abil 350 | Dimethicone | 0.5 | 2.0 | 1.0 | | | | | 0.5 |
| Allantoin | Allantoin | | 0.2 | 0.1 | | | | | |
| Aloe Vera Gel | Water (Aqua), Aloe | | | 3.0 | | | 3.0 | 0.45 | |
| Concentrate | Barbadensis Leaf Juice | | | | | | | | |
| 10/1* | | | | | | | | | |
| Alpinia Leaf | Alpinia Officinarum | | | | | 1.0 | | | |
| Extract Blend | Leaf Extract, | | | | | | | | |
| | Alpinia conchigera | | | | | | | | |
| | Leaf Extract, | | | | | | | | |
| | Alpinia Blepharocalyx | | | | | | | | |
| | Leaf Extract | | | | | | | | |
| Alugel 34 TH | Aluminium Stearate | | | | | | 1.0 | | |
| Aqua-Ceramide (Kao) | Cetyloxypropyl Glyceryl | | 0.1 | | | | | | |
| | Methoxypropyl Myristamide | | | | | | | | |
| Arbutin | β-Arbutin | 0.2 | | | | | | | |
| Butylene Glycol | Butylene Glycol | | | 5.0 | | | | | |
| Carbopol ETD 2050 | Carbomer | | | | | 0.2 | | | |
| Carbopol Ultrez-10 | Carbomer | | 0.1 | | | | | | |
| Ceramide 2 | Ceramide 2 | 0.1 | | | | | | | |
| Ceramide BIO* | Cetylhydroxyproline | | 0.1 | | | | | 0.2 | |
| | Palmitamide | | | | | | | | |
| Ceramide PC104 | Hydroxypropyl | | | | | 0.1 | | | |
| | Bispalmitamide MEA | | | | | | | | |
| Ceramide SL | Hydroxyethyl Palmityl | | | | | | 0.1 | | |
| | Oxyhydroxypropyl | | | | | | | | |
| | Palmitamide | | | | | | | | |
| Cetiol OE | Dicaprylyl Ether | | | 4.0 | | | | | |
| Cetiol SB 45 | Butyrospermum Parkii | | | 1.0 | | | | | |
| | (Shea Butter) | | | | | | | | |
| Citric Acid 10% sol. | Citric Acid | | | | | | | 0.3 | |
| Comperlan 100 | Cocamide MEA | | | | | | | 0.5 | |
| Crinipan AD | Climbazole | | | | | | | 0.5 | |
| Curcuma Extract | Curcuma Xanthorrhiza | | | | | | | | |
| | Root Extract | | | | | | | | |
| Curcuma Leaf Extract | Curcuma Aromatica | 1.0 | | | | | | | |
| | Leaf Extract | | | | | | | | |
| Curcuma Root Extract | Curcuma Longa | | 1.5 | | | | | | |
| | (Turmeric) Root Extract | | | | | | | | |
| Dehyquart A CA | Cetrimonium Chloride | | | | | | | | |
| Dehyquart SP | Quaternium-52 | | | | | | | | |
| Dihydroxyacetone | Dihydroxyacetone | | | | | | | | 5.0 |
| Dow Corning 246 Fluid | Cyclohexasiloxane and | | | | | 2.0 | | | |
| | Cyclopentasiloxane | | | | | | | | |
| Dow Corning 345 Fluid | Cyclomethicone | | | | 0.5 | | | | |
| D-Panthenol | Panthenol | | | 1.0 | | | | | |
| Dracorin ® CE* | Glyceryl Stearate | 5.0 | | | | | | | 5.0 |
| | Citrate | | | | | | | | |
| Dracorin ® GOC* | Glyceryl Oleate Citrate, | | | | 2.0 | | | | |
| | Caprylic/Capric | | | | | | | | |
| | Triglyceride | | | | | | | | |
| Drago-Beta-Glucan* | Water (Aqua), | 0.3 | | | | | | | |
| | Butylene Glycol, | | | | | | | | |
| | Glycerin, Avena | | | | | | | | |
| | Sativa (Oat), | | | | | | | | |
| | Kernel Extract | | | | | | | | |
| Dragoderm ®* | Glycerin, | | | | | | | 2.0 | |
| | Triticum Vulgare | | | | | | | | |
| | (Wheat) Gluten, Water | | | | | | | | |
| | (Aqua) | | | | | | | | |
| Drago-Oat-Active* | Water (Aqua), | | | | 1.0 | | | | |
| | Butylene Glycol, | | | | | | | | |
| | Avena Sativa | | | | | | | | |
| | (Oat) Kernel Extract | | | | | | | | |
| Dragosan W/O P* | Sorbitan Isostearate, | | | | | | 6.0 | | |
| | Hydrogenated Castor | | | | | | | | |
| | Oil, Ceresin, Beeswax | | | | | | | | |
| | (Cera Alba) | | | | | | | | |
| Dragosantol ® 100* | Bisabolol | 0.3 | | | 0.1 | 0.3 | 0.2 | | |
| Dragoxat ® 89* | Ethylhexyl | | | | | | | | |
| | Ethylisononanoate | | | | | | | | |
| EDETA B Powder | Tetrasodium EDTA | | | | | | | 0.1 | |
| EDETA BD | Disodium EDTA | | | | | 0.1 | | | 0.1 |
| Emulsiphos ®* | Potassium Cetyl Phosphate, | | 2.0 | | | 1.5 | | | |
| | Hydrogenated Palm | | | | | | | | |
| | Glycerides | | | | | | | | |
| Ethanol 96% | Ethanol | | | | | | | | 2.0 |
| Eumulgin B2 | Ceteareth-20 | | | | | | | | |
| Extrapone ® Green | Glycerin, Water (Aqua), | | 0.2 | | | | | | |
| Tea GW* | Camellia Sinensis | | | | | | | | |
| | Leaf Extract | | | | | | | | |
| Extrapone ® | Glycerin, Water (Aqua), | | 0.3 | | | | | | |
| Rosemary GW* | Rosmarinus officinalis | | | | | | | | |
| | (Rosemary) Leaf Extract | | | | | | | | |
| Extrapone ® Witch | Propylene Glycol, | | | | | | 1.0 | | |
| Hazel Distillate | Hamamelis Virginiana | | | | | | | | |
| colourless* | (Witch Hazel) | | | | | | | | |
| | Water, Water (Aqua), | | | | | | | | |
| | Hamamelis Virginiana | | | | | | | | |
| | (Witch Hazel) Extract | | | | | | | | |
| Farnesol* | Farnesol | | | | | | | | |
| Fragrance “Rose”* | Fragrance | | | | | | | | |
| Fragrance “WHITE”* | Fragrance | 0.3 | 0.3 | 0.3 | 0.2 | 0.4 | 0.4 | 0.5 | 0.3 |
| Frescolat ®MGA* | Menthone Glycerol Acetal | 0.5 | | | | 0.3 | | | |
| Frescolat ®ML cryst.* | Menthyl Lactate | | | 0.8 | | | | | |
| Frescolat ®X-COOL* | Menthyl Ethylamido Oxalate | | | | | | | | |
| Genapol LRO liquid | Sodium Laureth Sulfate | | | | | | | 37.0 | |
| Givobio GZN | Zinc Gluconate | | | | | | | | |
| Glycerol 85% | Glycerin | 3.0 | 2.0 | 4.0 | | 4.7 | 2.0 | | 1.5 |
| Glyceryl Stearate | Glyceryl Stearate | | 2.0 | | | | | | |
| Hydrolite ®-5* | Pentylene Glycol | | | | 5.0 | | | | 3.5 |
| Hydroviton ® 24* | Water, Glycerin, Sodium | | | | | | | | |
| | Lactate, TEA Lactate, | | | | | | | | |
| | Serine, Lactic Acid, | | | | | | | | |
| | Urea, Sorbitol, Sodium | | | | | | | | |
| | Chloride, Lauryl | | | | | | | | |
| | Diethylenedi- | | | | | | | | |
| | aminoglycine, | | | | | | | | |
| | Lauryl Aminopropyl- | | | | | | | | |
| | glycine, Allantoin | | | | | | | | |
| Hydroviton ® PLUS* | Water, Pentylene | | | 1.0 | | | | | |
| | Glycol, Glycerin, | | | | | | | | |
| | Fructose, Urea, | | | | | | | | |
| | Citric Acid, Sodium | | | | | | | | |
| | Hydroxide, Maltose, | | | | | | | | |
| | Sodium PCA, Sodium | | | | | | | | |
| | Chloride, Sodium | | | | | | | | |
| | Lactate, Trehalose, | | | | | | | | |
| | Allantoin, Sodium | | | | | | | | |
| | hyaluronate, Glucose | | | | | | | | |
| Irgasan DP 300 | Triclosan | | | | | | | | |
| Isoadipate ®* | Diisopropyl Adipate | | | | | | | | |
| Isodragol ®* | Triisononanoin | | 2.0 | | | | | | |
| Isopropyl Palmitate | Isopropyl Palmitate | 4.0 | | | | | | | 4.0 |
| Karion F | Sorbitol | | | | | | 2.0 | | |
| Keltrol RD | Xanthan Gum | 0.2 | 0.1 | | | 0.2 | | | 0.3 |
| Kojic acid | Kojic Acid | 1.0 | | | | | | | |
| Lanette 16 | Cetyl Alcohol | 1.0 | | | | | | | 1.0 |
| Lanette E | Sodium Cetearyl Sulfate | | | | | | | | |
| Lanette O | Cetearyl Alcohol | | 3.0 | | | 1.0 | | | |
| Lara Care A-200 | Galactoarabinan | | | 0.3 | | | | | |
| Magnesium Chloride | Magnesium | | | | | | 0.7 | | |
| | Chloride | | | | | | | | |
| Merquat 550 | Polyquaternium-7 | | | | | | | 0.5 | |
| NaOH 10% sol. | Sodium Hydroxide | | | | | | | | |
| Naringin | 4′,5,7- | | | | | | | 0.5 | 2.0 |
| | Trihydroxyflavone7- | | | | | | | | |
| | O-Neohesperidoside | | | | | | | | |
| Natrosol 250 HHR | Hydroxyethyl- | | | | | | | | |
| | cellulose | | | | | | | | |
| Neo Heliopan ® | Butyl Methoxy- | | | | | 1.0 | | | |
| 357* | dibenzoyl-methane | | | | | | | | |
| Neo Heliopan ® | Disodium Phenyl | | | | | 10 | | | |
| AP* (10% as | Dibenzimidazole | | | | | | | | |
| sodium salt) | Tetrasulfonate | | | | | | | | |
| Neo Heliopan ® | Ethylhexyl | 5.0 | | | | 3.0 | | | |
| AV* | Methoxy- | | | | | | | | |
| | cinnamate | | | | | | | | |
| Neo Heliopan ® | Isoamyl p- | | | | | | | | |
| E1000* | Methoxycinnamate | | | | | | | | |
| Neo Heliopan ® | Homosalate | | | | | | | | |
| HMS* | | | | | | | | | |
| Neo Heliopan ® | Phenylbenz- | | | | | 6.7 | | | |
| Hydro* | imidazole | | | | | | | | |
| (15% as sodium | Sulfonic Acid | | | | | | | | |
| salt) | | | | | | | | | |
| Neo Heliopan ® | 4-Methylbenzyl- | | | | | 1.5 | | | |
| MBC* | idene Camphor | | | | | | | | |
| Neo Heliopan ® | Ethylhexyl | | | | | 5.0 | | | |
| OS* | Salicylate | | | | | | | | |
| Neo PCL wssl. N* | Trideceth-9, PEG-5 | | | | | | | | |
| | Ethylhexanoate, | | | | | | | | |
| | Water | | | | | | | | |
| Neutral Oil | Caprylic/Capric | 6.0 | | | 4.0 | 2.0 | | | 6.0 |
| | Triglyceride | | | | | | | | |
| Oxynex 2004 | BHT | | | | | | 0.1 | | |
| Paraffin Oil | Paraffinum Liquidum | | | | 4.0 | | | | |
| PEG-5 | PEG-5 | | 1.42 | 0.15 | 2.84 | | 0.56 | 2.84 | |
| Isononanoate | Isononanoate | | | | | | | | |
| PEG-9 Tridecyl | Trideceth-9 | | 2.99 | 0.35 | 5.97 | | 1.2 | 5.97 | |
| Ether | | | | | | | | | |
| PCL Liquid 100* | Cetearyl | 3.0 | 5.0 | | 7.0 | | 12.0 | | 3.0 |
| | Ethylhexoate | | | | | | | | |
| PCL Solid* | Stearyl Heptanoate, | | 2.0 | | | | | | |
| | Stearyl Caprylate | | | | | | | | |
| Pemulen TR-2 | Acrylates/C10-30 | | | 0.3 | 0.2 | | | | |
| | Alkyl Acrylate | | | | | | | | |
| | Crosspolymer | | | | | | | | |
| Polymer JR 400 | Polyquaternium-10 | | | | | | | | |
| Polyquart H-81 | PEG-15 | | | | | | | | |
| | Cocopolyamine | | | | | | | | |
| Preservative | Phenoxyethanol, | | 0.8 | 0.7 | | 0.7 | 0.8 | | |
| | Methylparaben, | | | | | | | | |
| | Ethylparaben, | | | | | | | | |
| | Butylparaben, | | | | | | | | |
| | Propylparaben, | | | | | | | | |
| | Isobutylparaben | | | | | | | | |
| Propylene Glycol | Propylene Glycol | | 5.0 | | | | | | |
| Retinyl Palmitate in | Retinyl Palmitate | | | | | | 0.2 | | |
| Oil | | | | | | | | | |
| Sepigel 305 | Polyacrylamide, | | | | | | | | 1.0 |
| | C13-14 Isoparaffin, | | | | | | | | |
| | Laureth-7 | | | | | | | | |
| Sodium Ascorbyl | Sodium Ascorbyl | 2.0 | | 1.0 | | | | | |
| Phosphate | Phosphate | | | | | | | | |
| Sodium Benzoate | Sodium Benzoate | | | | | | | 0.5 | |
| Sodium Chloride | Sodium Chloride | | | | | | | 1.0 | |
| Sodium Hydroxide | Sodium Hydroxide | | 0.3 | 0.6 | 0.4 | | | | |
| (10% sol.) | | | | | | | | | |
| Solubilizer 611674* | PEG-40 | | | | | | | | |
| | Hydrogenated | | | | | | | | |
| | Castor Oil, | | | | | | | | |
| | Trideceth-9, Water | | | | | | | | |
| | (Aqua) | | | | | | | | |
| Sun Flower Oil | Helianthus Annuus | | | | | | 5.0 | | |
| | (Sunflower) Seed Oil | | | | | | | | |
| Sweet Almond Oil | Prunus dulcis | | | | | | 5.0 | | |
| SymCalmin ® | Pentylene Glycol, | | | 1.0 | | | | | |
| | Butylene Glycol, | | | | | | | | |
| | Hydroxyphenyl | | | | | | | | |
| | Propamidobenzoic | | | | | | | | |
| | Acid | | | | | | | | |
| Symdiol ®68* | 1,2-Hexanediol, | | | | | | | | |
| | Caprylylglycol, | | | | | | | | |
| Symdiol ®68T* | 1,2-Hexanediol, | 0.5 | | | | | | | |
| | Caprylylglycol, | | | | | | | | |
| | Tropolone | | | | | | | | |
| SymMatrix ®* | Maltodextrin, Rubus | | 0.1 | | | 0.3 | 1.0 | | |
| | Fruticosus | | | | | | | | |
| | (Blackberry) Leaf | | | | | | | | |
| | Extract | | | | | | | | |
| Symollient W/S* | Trideceth-9, PEG-5 | | | | | 2.94 | | | |
| (PEG-9 Tridecyl | Isononanoate, water | | | | | | | | |
| Ether, PEG-5 | | | | | | | | | |
| Isononanoate) | | | | | | | | | |
| SymMollient ®S* | Cetearyl Nonanoate | | | | | | | | |
| SymOcide ®PS* | Phenoxyethanol, | | | | | | | 1.0 | |
| | Decylene Glycol, | | | | | | | | |
| | 1,2 Hexanediol | | | | | | | | |
| Polyoxyethylene | Laureth-9 | | 0.5 | | | | | | |
| (9) Lauryl Ether | | | | | | | | | |
| SymRelief ® 100* | Bisabolol, Zingiber | | | | 0.1 | | | | |
| | Officinale (Ginger) | | | | | | | | |
| | Root Extract | | | | | | | | |
| SymRepair ®* | Hexyldecanol, | | | | | | | | |
| | Bisabolol, | | | | | | | | |
| | Cetylhydroxyproline | | | | | | | | |
| | Palmitamide, Stearic | | | | | | | | |
| | Acid, Brassica | | | | | | | | |
| | Campestris | | | | | | | | |
| | (Rapeseed) Sterols | | | | | | | | |
| SymSitive ®1609* | Pentylene Glycol, 4- | | | 1.5 | | | | | |
| | t-Butylcyclohexanol | | | | | | | | |
| SymSol ®PF3* | Water, Pentylene | | | | | | | | |
| | Glycol, Sodium | | | | | | | | |
| | Lauryl Sulfoacetate, | | | | | | | | |
| | Sodium Oleoyl | | | | | | | | |
| | Sarcosinate, | | | | | | | | |
| | Sodium Chloride, | | | | | | | | |
| | Disodium | | | | | | | | |
| | Sulfoacetate, | | | | | | | | |
| | Sodium Oleate, | | | | | | | | |
| | Sodium Sulfate | | | | | | | | |
| SymWhite ®377* | Phenylethyl- | 0.5 | | | | | | | |
| | resorcinol | | | | | | | | |
| Tego Betain L7 | Cocamidopropyl | | | | | | | 6.0 | |
| | Betaine | | | | | | | | |
| Tegosoft PC 31 | | | | | | | | | |
| Tegosoft TN | C12-15 Alkyl | | | 5.0 | | 5.0 | | | |
| | Benzoate | | | | | | | | |
| Texapon NSO BZ | Sodium laureth | | | | | | | | |
| | Sulfate | | | | | | | | |
| Tocopherol Acetate | Tocopheryl Acetate | | | 0.5 | | 0.5 | 3.0 | | |
| Triethanolamine, | Triethanolamine | | | | | 0.5 | | | |
| 99% | | | | | | | | | |
| Water, | Water (Aqua) | to 100 | to 100 | to 100 | to 100 | to 100 | to 100 | to 100 | to 100 |
| demineralized | | | | | | | | | |
| Zedoaria Leaf | Curcuma Zedoaria | 2.0 | | 1.5 | | | | | |
| Extract | Leaf Extract | | | | | | | | |
| Zingiber Extract | Zingiber Cassumunar | | | | | | | | |
| | Extract | | | | | | | | |
| Zirkonal L 450 | Aluminium Zirconium | | | | | | | | |
| | Pentachloro-hydrate | | | | | | | | |
| | (40% aqueous solution) | | | | | | | | |

| | % BY WEIGHT/FORMULATION EXAMPLE | | | | | | | |
|---|---|---|---|---|---|---|---|---|
| RAW MATERIAL NAME | 9 | 10 | 11 | 12 | 13 | 14 | 15 | 16 |
| 1,7-Bis(4-methoxyphenyl)- | 0.1 | 0.3 | 0.5 | 1.0 | | 0.06 | 2.0 | 0.01 |
| 3,5-heptanedione | | | | | | | | |
| 1,7-Bis-(4-hydroxy-3- | | | | | 0.02 | | | |
| methoxyphenyl)- | | | | | | | | |
| 3,5-heptanedione″ | | | | | | | | |
| Abil 350 | 0.5 | | 0.3 | | | | 0.1 | |
| Allantoin | 0.25 | | | | | | | |
| Aloe Vera Gel | | | | | | | | |
| Concentrate 10/1* | | | | | | | | |
| Alpinia Leaf | | 0.5 | | | | | | |
| Extract Blend | | | | | | | | |
| Alugel 34 TH | | | | | | | | |
| Aqua-Ceramide (Kao) | | 0.1 | | | | | | |
| Arbutin | | | | | | | | |
| Butylene Glycol | | | 3.0 | | | | | |
| Carbopol ETD 2050 | | | 0.2 | | 0.5 | | | |
| Carbopol Ultrez-10 | | | | | | | | |
| Ceramide 2 | | | | | | | | |
| Ceramide BIO* | 0.5 | | | | | | | |
| Ceramide PC104 | | | | | | | | |
| Ceramide SL | | | | | | | | |
| Cetiol OE | | | | | | | | |
| Cetiol SB 45 | | | | | | | | |
| Citric Acid 10% sol. | | | | | | | | |
| Comperlan 100 | | | | | | | | |
| Crinipan AD | | | | | | 0.5 | | |
| Curcuma Extract | | 0.5 | | | | | | |
| Curcuma Leaf Extract | | | | | | | | |
| Curcuma Root Extract | | | | | | | | |
| Dehyquart A CA | | | | | | | | 4.0 |
| Dehyquart SP | | | | | | | 0.5 | |
| Dihydroxyacetone | | | | | | | | |
| Dow Corning 246 Fluid | | | | | | | | |
| Dow Corning 345 Fluid | | | | | | | | |
| D-Panthenol | | | | | | | | 1.0 |
| Dracorin ® CE* | 1.5 | | | | | | | |
| Dracorin ® GOC* | | | | | | | | |
| Drago-Beta-Glucan* | | | | | | | | |
| Dragoderm ®* | | | | | | | | 2.0 |
| Drago-Oat-Active* | | | | | | | | |
| Dragosan W/O P* | | | | | | | | |
| Dragosantol ® 100* | | 0.1 | 0.1 | | 0.1 | | 0.1 | |
| Dragoxat ® 89* | 2.0 | | | 1.0 | 1.0 | | | |
| EDETA B Powder | | | | | | | | |
| EDETA BD | | | 0.1 | | 0.1 | | | |
| Emulsiphos ®* | 2.0 | | 1.5 | | | | | |
| Ethanol 96% | | 30.0 | | 13.0 | 5.0 | | | |
| Eumulgin B2 | | | | | | | 0.7 | |
| Extrapone ® Green | | | | | | | | |
| Tea GW* | | | | | | | | |
| Extrapone ® | 0.5 | | | | | | | |
| Rosemary GW* | | | | | | | | |
| Extrapone ® Witch | | | | | | | | |
| Hazel Distillate | | | | | | | | |
| colourless* | | | | | | | | |
| Farnesol* | | 0.5 | | | | | | |
| Fragrance “Rose”* | | | 0.3 | 0.5 | 0.4 | 0.3 | 0.5 | 0.1 |
| Fragrance “WHITE”* | 0.3 | 1.0 | | | | | | |
| Frescolat ®MGA* | | | | | | | | |
| Frescolat ®ML cryst.* | | 0.2 | | | | | | |
| Frescolat ®X-COOL* | | | 1.0 | | | | | |
| Genapol LRO liquid | | | | | | | | |
| Givobio GZN | 0.5 | | | | | | | |
| Glycerol 85% | 3.0 | | | 4.0 | | | | |
| Glyceryl Stearate | 2.0 | | 2.0 | | | | | |
| Hydrolite ®-5* | | | | | 4.5 | | | |
| Hydroviton ® 24* | 1.0 | | | | | | | |
| Hydroviton ® PLUS* | | | | | | | | |
| Irgasan DP 300 | | 0.3 | | | | | | |
| Isoadipate ®* | | | | | 1.0 | | | |
| Isodragol ®* | 3.0 | | | | | | | |
| Isopropyl Palmitate | | | | | | | | |
| Karion F | | | | | | | | |
| Keltrol RD | | | 0.2 | 0.2 | 0.3 | | | |
| Kojic acid | | | 0.5 | | | | | |
| Lanette 16 | | | 1.2 | | | | | |
| Lanette E | | | 0.7 | | | | | |
| Lanette O | 2.0 | | | | | | 2.5 | 1.5 |
| Lara Care A-200 | | | | | | | | |
| Magnesium Chloride | | | | | | | | |
| Merquat 550 | | | | | | | | |
| NaOH 10% sol. | 0.3 | | | | | | | |
| Naringin | | | | | | | | |
| Natrosol 250 HHR | | 0.3 | | | | | | |
| Neo Heliopan ® | | | | | 1.5 | 0.5 | | |
| 357* | | | | | | | | |
| Neo Heliopan ® | | | 22.0 | 25.0 | | | | |
| AP* (10% as | | | | | | | | |
| sodium salt) | | | | | | | | |
| Neo Heliopan ® | | | | | 5.0 | | | |
| AV* | | | | | | | | |
| Neo Heliopan ® | | | | | 5.0 | | | |
| E1000* | | | | | | | | |
| Neo Heliopan ® | | | 5.0 | | | | | |
| HMS* | | | | | | | | |
| Neo Heliopan ® | | | | 33.3 | 10.0 | 3.3 | | |
| Hydro* | | | | | | | | |
| (15% as sodium | | | | | | | | |
| salt) | | | | | | | | |
| Neo Heliopan ® | | | | | 2.0 | | | |
| MBC* | | | | | | | | |
| Neo Heliopan ® | | | | | | | | |
| OS* | | | | | | | | |
| Neo PCL wssl. N* | | | | | | | | 1.0 |
| Neutral Oil | 10.0 | | 2.0 | | | | | |
| Oxynex 2004 | | | | | | | | |
| Paraffin Oil | | | | | | | | |
| PEG-5 | 2.84 | | | | 0.28 | 0.84 | | 0.14 |
| Isononanoate | | | | | | | | |
| PEG-9 Tridecyl | | | | | 0.6 | | | 0.3 |
| Ether | | | | | | | | |
| PCL Liquid 100* | | | 3.0 | | | | 3.0 | |
| PCL Solid* | | | | | | | | |
| Pemulen TR-2 | | | | | | | | |
| Polymer JR 400 | | | | | | 0.4 | | |
| Polyquart H-81 | | | | | | | | 1.5 |
| Preservative | 0.8 | | | 0.8 | 0.8 | | | 0.8 |
| Propylene Glycol | | | | | | | | |
| Retinyl Palmitate in | | | | | | | | |
| Oil | | | | | | | | |
| Sepigel 305 | | | | | | | | |
| Sodium Ascorbyl | | | | | | | | |
| Phosphate | | | | | | | | |
| Sodium Benzoate | | | | | | | | |
| Sodium Chloride | | | | | | | | |
| Sodium Hydroxide | | | 2.8 | | 2.2 | | | |
| (10% sol.) | | | | | | | | |
| Solubilizer 611674* | | 2.0 | | | | 3.0 | | |
| Sun Flower Oil | | | | | | | | |
| Sweet Almond Oil | | | | | | | | |
| SymCalmin ® | 1.0 | | | | | | | |
| Symdiol ®68* | | | 0.5 | | | | | |
| Symdiol ®68T* | | | | | | | | |
| SymMatrix ®* | | | | | | | | |
| Symollient W/S* | | | | | | 1.8 | | |
| (PEG-9 Tridecyl | | | | | | | | |
| Ether, PEG-5 | | | | | | | | |
| Isononanoate) | | | | | | | | |
| SymMollient ®S* | | | 1.5 | | | | | |
| SymOcide ®PS* | | | | | | 1.0 | 1.0 | |
| Polyoxyethylene | | | | | | 1.0 | | |
| (9) Lauryl Ether | | | | | | | | |
| SymRelief ® 100* | | | | | | | | |
| SymRepair ®* | 2.0 | | | | | | | |
| SymSitive ®1609* | | | 0.5 | | | | | 1.5 |
| SymSol ®PF3* | | | | | 1.0 | | | |
| SymWhite ®377* | | | 0.5 | 1.0 | 1.0 | 1.0 | | |
| Tego Betain L7 | | | | | | | | |
| Tegosoft PC 31 | 0.3 | | | | | | | |
| Tegosoft TN | | | | | 4.0 | | | |
| Texapon NSO BZ | | | | | | 27.0 | | |
| Tocopherol Acetate | 0.3 | | 0.5 | | 0.5 | | | |
| Triethanolamine, | | | | | | | | |
| 99% | | | | | | | | |
| Water, | to 100 | to 100 | to 100 | to 100 | to 100 | to 100 | to 100 | to 100 |
| demineralized | | | | | | | | |
| Zedoaria Leaf | | | | | | | | |
| Extract | | | | | | | | |
| Zingiber Extract | 1.0 | | | | | | | |
| Zirkonal L 450 | | 37.0 | | | | | | |

*Symrise raw materials

The following Fragrance “WHITE” was used in Formulation Examples 1-10:

Fragrance “WHITE”: Perfume Oil with White Blossom Smell

| | Parts by |
|---|---|
| Component/NAME | weight |
| Benzyl acetate | 60.00 |
| Citronellyl acetate | 60.00 |
| Cyclamene aldehyde (2-methyl-3-(4-isopropylphenyl)propanal | 20.00 |
| Dipropylene glycol (DPG) | 60.00 |
| Ethyl linalool | 40.00 |
| Florol (2-isobutyl-4-methyltetrahydro-2H-pyran-4-ol) | 30.00 |
| Globanone [(E/Z)-8-cyclohexadecen-1-one] | 180.00 |
| Hedione (methyldihydrojasmonate) | 140.00 |
| Hexeny Isalicylate, cis-3 | 10.00 |

The following Fragrance “ROSE” was used in Formulation Examples 11-16:

Fragrance “ROSE”: Perfume oil with rose smell

| | Parts by |
|---|---|
| Component/NAME | weight |
| Acetophenone, 10% in DPG | 10.00 |
| n-undecanal | 5.00 |
| Aldehyde C14 so-called (peach aldehyde) | 15.00 |
| Allylamylglycolate, 10% in DPG | 20.00 |
| Amyl salicylate | 25.00 |
| Benzyl acetate | 60.00 |
| Citronellol | 80.00 |
| d-limonene | 50.00 |
| Decenol trans-9 | 15.00 |
| Dihydromyrcenol | 50.00 |
| Dimethylbenzylcarbinyl acetate | 30.00 |
| Diphenyloxide | 5.00 |
| Eucalyptol | 10.00 |
| Geraniol | 40.00 |
| Nerol | 20.00 |
| Geranium oil | 15.00 |
| Hexenol cis-3, 10% in DPG | 5.00 |
| Hexenyl salicylate cis-3 | 20.00 |
| Indole, 10% in DPG | 10.00 |
| Alpha-ionone | 15.00 |
| Beta-ionone | 5.00 |
| Lilial (2-methyl-3-(4-tert-butyl-phenyl)propanal) | 60.00 |
| Linalool | 40.00 |
| Methylphenylacetate | 10.00 |
| Phenylethyl alcohol | 275.00 |
| Styrallyl acetate | 20.00 |
| Terpineol | 30.00 |
| Tetrahydrolinalool | 50.00 |
| Cinnamyl alcohol | 10.00 |

### Formulation Examples 17-22

### Oral Hygiene/Orally Applicable Medicinal Products

The following Peppermint Flavor PF1 was used in Formulation Examples 17, 18 and 19:

| Peppermint Flavor PF1 | parts by weight |
|---|---|
| Isobutyraldehyde | 0.5 |
| 3-Octanol | 0.5 |
| Dimethyl sulphide | 0.5 |
| trans-2-Hexenal | 1.0 |
| cis-3-Hexenol | 1.0 |
| 4-Terpineol, natural | 1.0 |
| Isopulegol | 1.0 |
| Piperitone, natural, from eucalyptus | 2.0 |
| Linalool | 3.0 |
| 8-Ocimenyl acetate, 10% in triacetin | 5.0 |
| Isoamyl alcohol | 10.0 |
| Isovaleraldehyde | 10.0 |
| alpha-Pinene, natural | 25.0 |
| beta-Pinene, natural | 25.0 |
| Neomenthol, racemic | 40.0 |
| Eucalyptol (1,8-cineol), natural | 50.0 |
| L-Menthyl acetate of the formula D | 70.0 |
| L-Menthone | 220.0 |
| D-Isomenthone | 50.0 |
| L-Menthol | 483.5 |
| Nonenolide | 1.0 |

### Formulation Example 17

### Gel Dental Cream

| | I (%) | II (%) | III (%) |
|---|---|---|---|
| 1,7-Bis(4-methoxyphenyl)-3,5- | 0.1 | 0.01 | 0.06 |
| heptanedione | | | |
| Na carboxymethylcellulose | 0.40 | 0.40 | 0.40 |
| Sorbitol 70%, in water | 72.00 | 72.00 | 72.00 |
| Polyethylene glycol (PEG) 1500 | 3.00 | 3.00 | 3.00 |
| Na saccarinate | 0.07 | 0.07 | 0.07 |
| Na fluoride | 0.24 | 0.24 | 0.24 |
| p-Hydroxybenzoic acid (PHB) ethyl | 0.15 | 0.15 | 0.15 |
| ester | | | |
| Peppermint flavor PF1 | 1.00 | 1.00 | 1.00 |
| Abrasive silica | 11.00 | 11.00 | 11.00 |
| Thickening silica | 6.00 | 6.00 | 6.00 |
| Sodium dodecyl sulfate (SDS) | 1.40 | 1.40 | 1.40 |
| PEG-9 Tridecyl Ether | 2.99 | 0.3 | 1.8 |
| PEG-5 Isononanoate | 1.42 | 0.14 | 0.84 |
| Dist. water | to 100.00 | to 100.00 | to 100.00 |

### Formulation Example 18

### Dental Cream Against Plaque

| | I (%) | II (%) | III (%) |
|---|---|---|---|
| 1,7-Bis-(4-hydroxy-3- | 0.06 | 0.1 | 0.3 |
| methoxyphenyl)-3,5-heptanedione | | | |
| Carrageenan | 0.90 | 0.90 | 0.90 |
| Glycerin | 15.00 | 15.00 | 15.00 |
| Sorbitol 70%, in water | 25.00 | 25.00 | 25.00 |
| PEG 1000 | 3.00 | 3.00 | 3.00 |
| Na fluoride | 0.24 | 0.24 | 0.24 |
| Tetrapotassium diphosphate | 4.50 | 4.50 | 4.50 |
| Tetrasodium diphosphate | 1.50 | 1.50 | 1.50 |
| Na saccarinate | 0.40 | 0.40 | 0.40 |
| Precipitated silica | 20.00 | 20.00 | 20.00 |
| Titanium dioxide | 1.00 | 1.00 | 1.00 |
| PHB methyl ester | 0.10 | 0.10 | 0.10 |
| Spearmint flavor (comprising 60 | 1.00 | 1.10 | 1.20 |
| wt. % l-carvone and 25 wt. % | | | |
| l-menthol) | | | |
| PEG-9 Tridecyl Ether | 1.8 | 2.99 | |
| PEG-5 Isononanoate | 0.84 | 1.42 | |
| Sodium dodecyl sulfate | 1.30 | 1.30 | 1.30 |
| Dist. water | to 100.00 | to 100.00 | to 100.00 |

### Formulation Example 19

### Dental Cream Against Sensitive Teeth

| | I (%) | II (%) | III (%) |
|---|---|---|---|
| 1,7-Bis(4-methoxyphenyl)-3,5- | 0.1 | 0.3 | 0.5 |
| heptanedione | | | |
| PEG-9 Tridecyl Ether | | | |
| PEG-5 Isononanoate | | | |
| Na carboxymethylcellulose | 0.70 | 0.70 | 0.70 |
| Xanthan gum | 0.50 | 0.50 | 0.50 |
| Glycerin | 15.00 | 15.00 | 15.00 |
| Sorbitol 70%, in water | 12.00 | 12.00 | 12.00 |
| K-nitrate | 5.00 | 5.00 | 5.00 |
| Na monofluorophosphate | 0.80 | 0.80 | 0.80 |
| PHB methyl ester | 0.15 | 0.15 | 0.15 |
| PHB propyl ester | 0.05 | 0.05 | 0.05 |
| Na saccharinate | 0.20 | 0.20 | 0.20 |
| Peppermint flavor PF1 | 1.00 | 1.00 | 1.00 |
| Ca-carbonate | 35.00 | 35.00 | 35.00 |
| Silicon dioxide | 1.00 | 1.00 | 1.00 |
| Sodium dodecyl sulfate (SDS) | 1.50 | 1.50 | 1.50 |
| Dist. water | to 100.00 | to 100.00 | to 100.00 |

### Formulation Example 20

### Ready-to-Use Mouthwash with Fluoride

| | I (%) | II (%) | III (%) |
|---|---|---|---|
| 1,7-Bis(4-methoxyphenyl)-3,5- | 0.1 | 0.01 | 0.06 |
| heptanedione | | | |
| Ethanol | 7.00 | 7.00 | 7.00 |
| Glycerin | 12.00 | 12.00 | 12.00 |
| Na fluoride | 0.05 | 0.05 | 0.05 |
| Pluronic F-127 ® (BASF, | 1.40 | 1.40 | 1.40 |
| surface-active substance) | | | |
| Na phosphate buffer pH 7.0 | 1.10 | 1.10 | 1.10 |
| Sorbic acid | 0.20 | 0.20 | 0.20 |
| Na saccharinate | 0.10 | 0.10 | 0.10 |
| Cinnamon/menthol aroma | 0.15 | 0.15 | 0.15 |
| PEG-9 Tridecyl Ether | 2.99 | 0.3 | 1.8 |
| PEG-5 Isononanoate | 1.42 | 0.14 | 0.84 |
| Dyestuff | 0.01 | 0.01 | 0.01 |
| Dist. water | to 100 | to 100 | to 100 |

### Formulation Example 21

### Sugar-Free Chewing Gum

| | I (%) | II (%) | III (%) |
|---|---|---|---|
| 1,7-Bis-(4-hydroxy-3- | 0.04 | 0.03 | 0.1 |
| methoxyphenyl)-3,5-heptanedione | | | |
| Chewing gum base | 30.00 | 30.00 | 30.00 |
| Sorbitol, powder | Ad 100.00 | Ad 100.00 | Ad 100.00 |
| Palatinite | 9.50 | 9.50 | 9.50 |
| Xylitol | 2.00 | 2.00 | 2.00 |
| Mannitol | 3.00 | 3.00 | 3.00 |
| Aspartame | 0.10 | 0.10 | 0.10 |
| Acesulfame K | 0.10 | 0.10 | 0.10 |
| Emulgum/emulsifier | 0.30 | 0.30 | 0.30 |
| Sorbitol 70%, in water | 14.00 | 14.00 | 14.00 |
| PEG-9 Tridecyl Ether | 1.2 | 0.9 | 2.99 |
| PEG-5 Isononanoate | 0.56 | 0.42 | 1.42 |
| Glycerin | 1.00 | 1.00 | 1.00 |
| Peppermint flavor PF1 | 1.50 | 1.50 | 1.50 |

### Formulation Example 22

### Compressed Tablets

| | I (wt. %) | II (wt. %) | III (wt. %) |
|---|---|---|---|
| 1,7-Bis(4-methoxyphenyl)-3,5- | 0.1 | 0.01 | 0.06 |
| heptanedione | | | |
| PEG-9 Tridecyl Ether | 2.99 | 0.3 | 1.8 |
| PEG-5 Isononanoate | 1.42 | 0.14 | 0.84 |
| Magnesium stearate (as lubricant) | 0.90 | 0.90 | 0.90 |
| Citric acid | 0.20 | 0.20 | 0.20 |
| Dextrose | Ad 100 | Ad 100 | Ad 100 |

### Formulation Examples 23-27

### Deo/Antiperspirant Formulations, Microemulsion

### Formulation Example 23

### Deodorant Sticks

| | A % | B % |
|---|---|---|
| Component/NAME | by weight | by weight |
| 1,7-Bis(4-methoxyphenyl)-3,5-heptanedione | 0.1 | 0.01 |
| Sodium stearate | 8.00 | 8.00 |
| PPG-3 Myristyl ether | 70.00 | 70.00 |
| PEG-9 Tridecyl Ether | 2.99 | 0.3 |
| PEG-5 Isononanoate | 1.42 | 0.14 |
| 1,2-propylene glygol | 10.00 | 10.00 |
| 1,1-dimethyl-3-phenylpropanol | 0.20 | 0.25 |
| Fragrance “WHITE” | 0.55 | — |
| Fragrance “ROSE” | — | 0.65 |
| Water | Ad 100 | Ad 100 |

### Formulation Example 24

### Microemulsion Gels

| Component/NAME | I (wt. %) | II (wt. %) |
|---|---|---|
| 1,7-Bis-(4-hydroxy-3-methoxyphenyl)-3,5- | 0.02 | 0.1 |
| heptanedione | | |
| Glycerin isostearate | 1.80 | 2.00 |
| Octoxyglycerin | 1.00 | 0.80 |
| Ceteareth-15 | 5.20 | 5.00 |
| PEG-150 Distearate | 1.00 | 1.00 |
| Aluminium chlorohydrate | 5.00 | 5.00 |
| Isotridecylisononanoate | 3.30 | 3.50 |
| Cyclomethicone | 6.60 | 6.40 |
| PEG-9 Tridecyl Ether | 0.6 | 2.99 |
| PEG-5 Isononanoate | 0.28 | 1.42 |
| Fragrance “WHITE” | 0.55 | — |
| Fragrance “ROSE” | — | 0.60 |
| Water | Ad 100 | Ad 100 |

### Formulation Example 25

### Antiperspirant Formulations

| Component/NAME | I (wt. %) | II (wt. %) |
|---|---|---|
| 1,7-Bis(4-methoxyphenyl)-3,5-heptanedione | 0.02 | 0.1 |
| Reach AZP-908 SUF | 24.00 | 22.00 |
| Cyclomethicone (Pentamer) | Ad 100 | Ad 100 |
| Polydecene (Silkflo 364 NF) | 17.50 | 20.00 |
| Neo Helipan OS (ethylhexyl salicylate, | 2.50 | 1.00 |
| Symrise AG) | | |
| L-Menthyl lactate (Frescolate ML, Symrise AG) | 0.25 | — |
| Polyethylene | 3.00 | 3.00 |
| Hydrogenated caster oil | 2.00 | 2.00 |
| Promyristyl PM-3 | 7.00 | 7.00 |
| PEG-9 Tridecyl Ether | 0.6 | 2.99 |
| PEG-5 Isononanoate | 0.28 | 1.42 |
| PEG-8 Distearate | 3.00 | 3.00 |
| Silicon dioxide (Cab-O-Sil M-5) | 1.00 | 1.00 |
| Stearyl alcohol | 15.00 | 10.00 |
| Octyldodecanol | — | 8.00 |
| Trans-4-tert-butyl cyclohexanol | 0.80 | 1.05 |
| Fragrance “WHITE” | 0.75 | — |
| Fragrance “ROSE” | — | 0.80 |

### Formulation Example 26

### Suspension Sticks

| Component/NAME | I (wt. %) | II (wt. %) | III (wt. %) |
|---|---|---|---|
| 1,7-Bis(4-methoxyphenyl)-3,5- | 0.1 | 0.01 | 0.06 |
| heptanedione | | | |
| Stearyl alcohol | 20.00 | 20.00 | 20.00 |
| Cyclomethicone | Ad 100 | Ad 100 | Ad 100 |
| PPG-14 Butylether | 2.00 | 2.00 | 2.00 |
| Hydrogenated caster oil | 1.00 | 1.00 | 1.00 |
| Talc | 2.00 | 2.00 | 2.00 |
| Aluminium chlorohydrate, powder | 20.00 | 20.00 | 20.00 |
| PEG-9 Tridecyl Ether | 2.99 | 0.3 | 1.8 |
| PEG-5 Isononanoate | 1.42 | 0.14 | 0.84 |
| Triclosan ® (5-chloro-2-(2,4- | 0.30 | — | 0.30 |
| dichlorophenoxy)phenol) | | | |
| Ethylhexylglycerin (Octoxyglycerin) | 0.50 | 0.80 | 0.50 |
| 1,1-Dimethyl-3-phenylpropanol | 0.30 | 0.40 | 0.35 |
| Anis alcohol | — | — | 0.15 |
| Trans-4-tert-butyl cyclohexanol | 0.55 | 0.85 | 1.10 |
| Fragrance “WHITE” | 0.55 | — | 0.25 |
| Fragrance “ROSE” | — | 0.70 | 0.45 |

### Formulation Example 27

### Deodorant Sprays

| Component/NAME | I (wt. %) | II (wt. %) | III (wt. %) |
|---|---|---|---|
| 1,7-Bis(4-methoxyphenyl)-3,5- | 0.1 | 0.01 | 0.06 |
| heptanedione | | | |
| PEG-9 Tridecyl Ether | 2.99 | 0.3 | 1.8 |
| PEG-5 Isononanoate | 1.42 | 0.14 | 0.84 |
| PEG-40-hydrogenated caster oil | 3.00 | 3.00 | 3.00 |
| Ethylhexylglycerin (Octoxyglycerin) | 0.80 | 0.80 | 0.80 |
| Ethanol | 40.00 | 40.00 | 40.00 |
| Citrate buffer | 0.50 | 0.50 | 0.50 |
| Phenoxyethanol | 0.25 | 0.35 | — |
| Triclosan ® (5-chloro-2-(2,4- | 0.25 | — | — |
| dichlorophenoxy)phenol) | | | |
| 2-Benzylheptan-1-ol (Jasmol) | — | 0.05 | 0.15 |
| Fragrance “WHITE” | 0.55 | — | 0.25 |
| Fragrance “ROSE” | — | 0.70 | 0.45 |
| Water | Ad 100 | Ad 100 | Ad 100 |

### Formulation Examples 28-34

### Hair Coloration, Hair Relaxer

### Formulation Example 28

### Liquid Gel Coloration with MEA

| | Formula 1 |
|---|---|
| Raw material | % weight |
| 1,7-Bis(4-methoxyphenyl)-3,5-heptanedione | 0.01 |
| PEG-9 Tridecyl Ether | 0.3 |
| PEG-5 Isononanoate | 0.14 |
| Butylene Glycol, Laureth-8, Laureth-3, | 60.00 |
| Cetrimonium, Chloride, Trideceth-2 | |
| carboxamide MEA, Butoxyethanol & Oleyl alcohol | |
| Butylene Glycol | 4.00 |
| Sodium Acetate | 1.00 |
| Hair dye | according to shade |
| Monoethanolamine (MEA) | according to shade |
| Fragrance | 0.5 |
| Water (Aqua) | QS 100 |

### Formulation Example 29

### Hair Coloration Cream with Ammonia

| | Formula 2 |
|---|---|
| Raw material | % weight |
| 1,7-Bis(4-methoxyphenyl)-3,5-heptanedione | 0.02 |
| PEG-9 Tridecyl Ether | 0.6 |
| PEG-5 Isononanoate | 0.28 |
| Cetyl alcohol, Oleyl alcohol, Cetearyl alcohol, | 21.00 |
| Stearic acid | |
| Ceteareth-25 | 4.00 |
| Laureth-8 | 10.00 |
| Sodium Cetearyl Sulfate | 1.00 |
| Polyquaternium-6 | 4.00 |
| Hair dye | according to shade |
| Ammonia (aqueous solution 20%) | according to shade |
| Fragrance | 0.50 |
| Water (Aqua) | QS 100 |

### Formulation Example 30

### Hair Coloration Developer Cream with Hydrogen Peroxide

| | Formula 3 |
|---|---|
| Raw material | % weight |
| 1,7-Bis(4-methoxyphenyl)-3,5-heptanedione | 0.04 |
| PEG-9 Tridecyl Ether | 1.2 |
| PEG-5 Isononanoate | 0.56 |
| Cetyl Alcohol, Ceteareth-25 | 7.00 |
| Sodium Stannate | 0.05 |
| Sodium Pyrophosphate | 0.05 |
| Pentasodium Pentetate | 0.30 |
| Hydrogen Peroxide (Hydrogen Peroxide 110 vol. | 20.00 |
| (aqueous solution 30%) | |
| Fragrance | 0.30 |
| Water (Aqua) | QS 100 |

### Formulation Example 31

### Hair Relaxer Lotion with Thioglycolate

| | Formula 4 |
|---|---|
| Raw material | % weight |
| 1,7-Bis(4-methoxyphenyl)-3,5-heptanedione | 0.03 |
| PEG-9 Tridecyl Ether | 0.9 |
| PEG-5 Isononanoate | 0.42 |
| Ammonium Thioglycolate | 9.00 |
| Oleth-20 | 0.50 |
| Phenoxyethanol, Methylparaben, Ethylparaben, | 0.50 |
| Butylparaben, Popylparaben, Isobutylparaben | |
| Fragrance | 0.3 |
| Water (Aqua) | QS 100 |

### Formulation Example 32

### Hair Relaxer Lotion with Sodium Hydroxide

| | Formula 5 |
|---|---|
| Raw material | % weight |
| 1,7-Bis(4-methoxyphenyl)-3,5-heptanedione | 0.06 |
| PEG-9 Tridecyl Ether | 1.8 |
| PEG-5 Isononanoate | 0.84 |
| Cetearyl alcohol, Dicethyl Phosphate, Ceteth-10 Phosphate | 7.00 |
| Cetearyl Alcohol | 2.00 |
| Petrolatum | 15.00 |
| Paraffinum Liquidum | 15.00 |
| Propylene Glycol | 3.00 |
| Phenoxyethanol, Methylparaben, Ethylparaben, | 0.50 |
| Butylparaben, Popylparaben, Isobutylparaben | |
| Sodium Hydroxide ((aqueous solution 20%) | 10.00 |
| Fragrance | 0.80 |
| Water (Aqua) | QS 100 |

### Formulation Example 33

### Hair Relaxer Cream Base with Calcium Hydroxide

| | Formula 6 |
|---|---|
| Raw material | % weight |
| 1,7-Bis(4-methoxyphenyl)-3,5-heptanedione | 0.1 |
| PEG-9 Tridecyl Ether | 2.99 |
| PEG-5 Isononanoate | 1.42 |
| Polawax NF | 16.48 |
| Cetearyl Alcohol | 1.96 |
| Oleth-10 | 4.00 |
| Petrolatum | 7.00 |
| Paraffinum Liquidum | 7.50 |
| Propylene Glycol | 2.00 |
| Phenoxyethanol, Methylparaben, Ethylparaben, | 0.80 |
| Butylparaben, Popylparaben, Isobutylparaben | |
| Calcium Hydroxide | 5.00 |
| Fragrance | 0.80 |
| Water (Aqua) | QS 100 |

### Formulation Example 34

### Hair Relaxer Liquid Activator with Guanidine Carbonate

| | Formula 6 bis |
|---|---|
| Raw material | % weight |
| 1,7-Bis(4-methoxyphenyl)-3,5-heptanedione | 0.04 |
| PEG-9 Tridecyl Ether | 1.2 |
| PEG-5 Isononanoate | 0.56 |
| Guanidine Carbonate | 25.00 |
| Triethylene Glycol, Imidazolidinyl Urea, | 0.20 |
| Methylparaben, Propylparaben, | |
| Dehydroacetic Acid | |
| Water (Aqua) | QS 100 |

1-18. (canceled)

19. A method for preventing, reducing or alleviating itchy skin conditions comprising administering to an individual in need thereof a compound of formula 1 or a salt thereof: 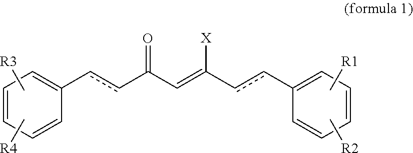
wherein, R1, R2, R3 and R4 are independently selected from —OH, —OR or —OCOR;
R is an alkyl group comprising 1-12 carbon atoms; and
X is —H or —OH.

20. The method according to claim 19, wherein the compound has a structure according to formula 2 or a salt thereof: 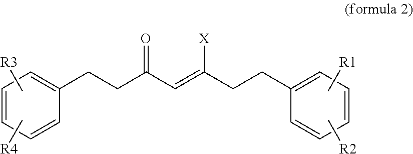
Wherein, R1, R2, R3 and R4 and X are as defined in claim 19.

21. The method according to claim 19, wherein the compound has a structure according to formula 3 or a salt thereof:

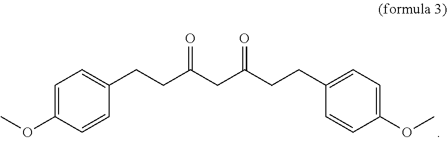

22. The method according to claim 19, wherein the compound has a structure according to formula 4 or a salt thereof:

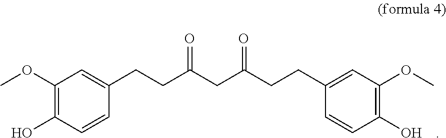

23. The method according to claim 19, further comprising administering one or more substances selected from the group consisting of:
(i) anti-itch compounds,
(ii) steroidal anti-inflammatory substances of the corticosteroid type,
(iii) non-steroidal anti-inflammatory substances,
(iv) natural or naturally occurring anti-inflammatory substances or substances that alleviate reddening and/or itching,
(v) alpha-bisabolol, apigenin, apigenin-7-glucoside, gingerols, shogaols, gingerdiols, dehydrogingerdiones, paradols, natural avenanthramides, non-natural avenanthramides, preferably dihydroavenanthramide D, boswellic acid, phytosterols, glycyrrhizin, glabridin and licochalcone A,
(vi) skin care agents,
(vii) physiological cooling agents, and
(viii) histamine receptor antagonists, serine protease inhibitors, TRPV1 antagonists, NK1 antagonists, cannabinoid receptor agonists and TRPV3 antagonists.

24. The method according to claim 19, wherein said one or more compounds of formula 1 and/or salt(s) thereof is present in an amount sufficient to prevent, reduce or alleviate one or more itchy skin conditions.

25. The method according to claim 19, wherein the compound(s) and/or salt(s) thereof, is applied to skin, and remains on the skin for at least 5 minutes.

26. A method for antagonizing PAR-2 in an individual in need thereof comprising administering a compound of formula 1 or a salt thereof: 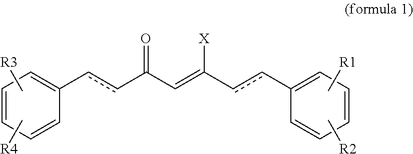
wherein, R1, R2, R3 and R4 are independently selected from —OH, —OR or —OCOR;
R is an alkyl group comprising 1-12 carbon atoms; and
X is —H or —OH.

27. The method according to claim 26, wherein the compound has a structure according to formula 2 or a salt thereof: 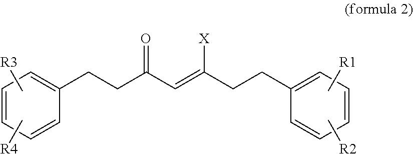
wherein R1, R2, R3 and R4 and X are as defined in claim 26.

28. The method according to claim 26, wherein the compound has a structure according to formula 3 or a salt thereof:

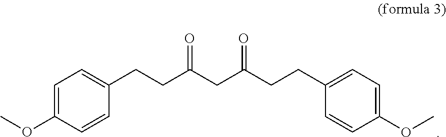

29. The method according to claim 26, wherein the compound has a structure according to formula 4 or a salt thereof:

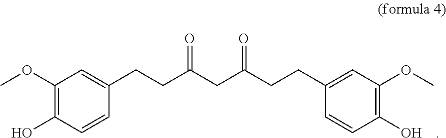

30. The method according to claim 26, wherein the composition comprising the compound(s) and/or salt(s) thereof, is applied to skin, and remains on the skin for at least 5 minutes.

31. A composition comprising:
a) from 0.01 to 10.0 wt. %, based on the total weight of said composition, of one or more compounds according to formula 1: 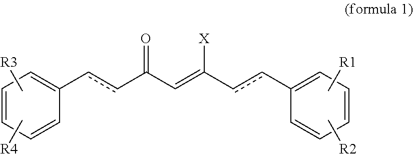
Wherein, R1, R2, R3 and R4 are independently selected from —OH, —OR or —OCOR;
R is an alkyl group comprising 1-12 carbon atoms; and
X is —H or —OH, and
b) one or more cosmetically and/or pharmaceutically acceptable carriers.

32. The composition according to claim 31, wherein the compound has a structure according to formula 2 or a salt thereof: 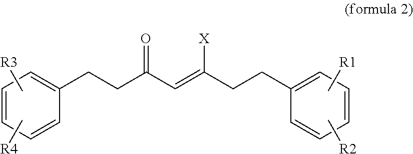
wherein R1, R2, R3 and R4 and X are as defined in claim 31.

33. The composition according to claim 31, wherein the compound has a structure according to formula 3 or a salt thereof:

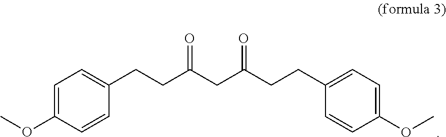

34. The composition according to claim 31, wherein the compound has a structure according to formula 4 or a salt thereof:

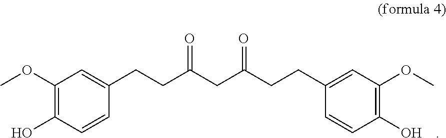

35. The composition according to claim 31, wherein the cosmetically and/or pharmaceutically acceptable carrier(s) comprise one or more of:
(i) polyethylene glycol esters of formula 5 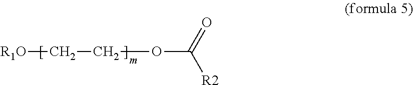
wherein m is an integer from 3-7,
R1 is H or 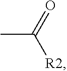
and
R2 is a branched or unbranched alkyl group,
(ii) polyethyleneglycol ethers of formula 6
R₃OCH₂—CH₂ₙOR₄ (formula 6)
wherein m is an integer from 7-30, and
R3 and R4 are independently H or a saturated or unsaturated, branched or unbranched alkyl group,
(iii) (alkane) diols having 3 to 10 carbon atoms,
(iv) esters having 6 to 36 carbon atoms,
(v) branched and unbranched alkyl or alkenyl alkohols,
(vi) branched and unbranched hydrocarbons and waxes, cyclic or linear silicone oils and dialkyl ethers having 6 to 24 carbon atoms, and
(vii) solvents selected from the group consisting of acetone, methylpropyl ketone, dipropyl ketone, dimethyl sulfoxide, glycerine carbonate, propylene carbonate, butylene carbonate, glycerine formal, solketal, 2-etyhl hexanol, 2-butyl octanol, 2-hexyl decanol and 2-octyl dodecanol.

36. The composition according to claim 31, further comprising one or more substances selected from the group consisting of:
(i) anti-itch compounds,
(ii) steroidal anti-inflammatory substances of the corticosteroid type,
(iii) non-steroidal anti-inflammatory substances,
(iv) natural or naturally occurring anti-inflammatory substances or substances that alleviate reddening and/or itching,
(v) alpha-bisabolol, apigenin, apigenin-7-glucoside, gingerols, shogaols, gingerdiols, dehydrogingerdiones, paradols, natural avenanthramides, non-natural avenanthramides, preferably dihydroavenanthramide D, boswellic acid, phytosterols, glycyrrhizin, glabridin and licochalcone A,
(vi) skin care agents,
(vii) physiological cooling agents, and
(viii) histamine receptor antagonists, serine protease inhibitors, TRPV1 antagonists, NK1 antagonists, cannabinoid receptor agonists or TRPV3 antagonists.

37. The composition according claim 31, further comprising one or more substances selected from the groups consisting of:
extracts or fractions from camomile, Aloe vera, oats, calendula, arnica, honeysuckle, rosemary, witch hazel, ginger or Echinacea, alpha-bisabolol, gingerols, shogaols, gingerdiols, dehydrogingerdiones, paradols, natural avenanthramides, non-natural avenanthramides, preferably dihydroavenanthramide D, boswellic acid, phytosterols, glycyrrhizin, and licochalcone A, urea, hyaluronic acid, allantoin, panthenol, lanolin, alpha-hydroxy acids (preferably citric acid, lactic acid), vitamin E and derivatives thereof.

38. The composition according to claim 31, wherein the composition is in the form of alcoholic or aqueous/alcoholic solution, dispersion, suspension, emulsion, ointment, paste, gel, balm, serum, powder, wipe, Eau de Toilette, Eau de Cologne, perfume, stick, roll-on, (pump) spray, aerosol, leave-on skin care composition, leave-on insect repellent composition, sunscreen composition, skin-lightening composition, self-tanning composition, aftersun preparation, shaving or after-shave composition, hair-removing composition, hair care composition, preferably conditioner, hair lotion, hair tonic, styling cream, pomade, styling aid, permanent wave and fixing compositions, hair smoothing composition, hair straightening composition, hair relaxer, hair setting composition, blonding composition, hair coloring composition, such as e.g. temporary, directly absorbed, semi-permanent hair coloring composition, permanent hair coloring composition, decorative cosmetic composition, deodorant and/or antiperspirant composition.

39. A pharmaceutical composition comprising:
(i) from 0.01 to 10 wt. % based on the total weight of said composition, one or more compounds according to formula 1: 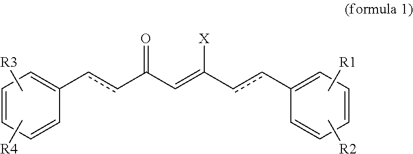
wherein R1, R2, R3 and R4 are independently selected from —OH, —OR or —OCOR;
R is an alkyl group comprising 1-12 carbon atoms; and
X is —H or —OH; and
(ii) a pharmaceutically acceptable carrier.

40. The pharmaceutical composition according to claim 39, wherein the compound has a structure according to formula 2 or a salt thereof: 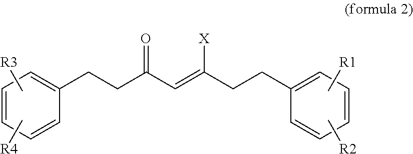
wherein R1, R2, R3 and R4 and X are as defined in claim 39.

41. The pharmaceutical composition according to claim 39, wherein the compound has a structure according to formula 3 or a salt thereof:

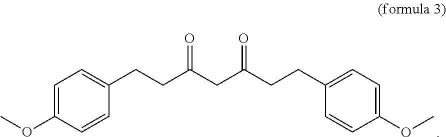

42. The pharmaceutical composition according to claim 39, wherein the compound has a structure according to formula 4 or a salt thereof:

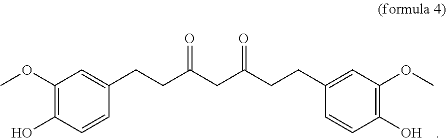

43. The pharmaceutical composition according to claim 39, wherein said one or more compound(s) of formula 1 and/or salt(s) thereof is present in an amount sufficient to a) prevent, reduce or alleviate one or more itchy skin conditions, and/or to b) provide a PAR-2 antagonistic effect.
